(19) United States
(12) Patent Application Publication
(10) Pub. No.: US 2024/0385200 A1
(43) Pub. Date: Nov. 21, 2024

(54) COMPOSITIONS AND METHODS OF DETERMINING PH AND POTASSIUM CONCENTRATION IN SAMPLES

(71) Applicant: The University of Chicago, Chicago, IL (US)

(72) Inventors: Yamuna Krishnan, Chicago, IL (US); Anees Palapuravan, Chicago, IL (US)

(21) Appl. No.: 18/667,104

(22) Filed: May 17, 2024

Related U.S. Application Data
(60) Provisional application No. 63/503,033, filed on May 18, 2023.

(51) Int. Cl. G01N 33/84; G01N 33/53; G01N 33/542

(52) U.S. Cl. CPC G01N 33/84; G01N 33/5308; G01N 33/542

(57) ABSTRACT

Compositions and methods for simultaneous determination of pH and potassium (K+) concentration in biological samples are provided. The methods employ labeled nucleic acid complexes formed by the hybridization of four single stranded nucleic acid molecules.

### CROSS-REFERENCE TO RELATED APPLICATIONS

This application claims the benefit of priority of U.S. Provisional Application No. 63/503,033, filed May 18, 2023, which is incorporated herein by reference in its entirety.

### REFERENCE TO AN ELECTRONIC SEQUENCE LISTING

This application contains a Sequence Listing which has been submitted electronically and is hereby incorporated by reference in its entirety. The Sequence Listing was created on Jun. 13, 2024, is named “23-0475-US_SequenceListing_ST26” and is 16,129 bytes in size.

### BACKGROUND

### Field of Disclosure

This disclosure relates to compositions and methods for determining pH and also potassium (K⁺) concentration in biological samples. More particularly, this disclosure relates to nucleic acid complexes and methods capable of simultaneously determining pH and K⁺ concentration using the nucleic acid complexes.

### Technical Background

Cell surface potassium ion (K⁺) channels regulate nutrient transport, cell migration and intercellular communication by regulating K⁺ permeability across the plasma membrane^{1,2}.

The cell surface K⁺ channels are assembled in the endoplasmic reticulum (ER) and trafficked to the plasma membrane by organelles³. The trans-Golgi network (TGN) exports fully competent K⁺ channels to the plasma membrane while early endosomes (EE) sort and deliver endocytosed K⁺ channels to recycling endosomes (RE) for return to the plasma membrane. Recently, it was shown that these organelles have membrane potentials that could gate various voltage-gated channels⁵, raising the possibility that cell surface K⁺ channels are active in organelles. G-protein coupled receptors (GPCRs) were also formerly thought to be active only at the plasma membrane⁶. However, a conformation-specific fluorescent reporter of GPCRs revealed that they were also activated in organelles by their ligands. Though K⁺ channels adopt well-defined activated and inactivated states⁷, conformation-specific reporters of K⁺ channels do not exist. That is, there are no known fluorescent reporters of K⁺ for acidic organelle lumens. Such reporters would be highly desirable by providing a way to map the organelle-specific activity of K⁺ channels, help identify new organellar K⁺ channels or channel modulators with nuanced functions, and facilitate the development of new treatments for medical conditions resulting from impaired potassium ion channel activity.

### SUMMARY

The inventors have determined that the novel nucleic acid complexes of the disclosure can efficiently and accurately determine pH in addition to K⁺ concentration in samples. In certain embodiments, the novel nucleic acid complexes of the disclosure simultaneously determine pH and K⁺ concentration in samples.

In one aspect, a method for simultaneously determining pH and K⁺ concentration in a sample is provided. The method comprises:
providing a nucleic acid complex comprising:
a first single-stranded nucleic acid molecule (D_{K}) comprising a K⁺ fluorophore conjugated to the first single-stranded nucleic acid molecule, the first single-stranded nucleic acid molecule including a first portion and a second portion;
a second single-stranded nucleic acid molecule (D_{D}) comprising a first label of a fluorescence resonance energy transfer (FRET) pair conjugated thereto, the second single-stranded nucleic acid molecule comprising a first portion and a second portion, wherein the second portion of the second single-stranded nucleic acid molecule is complementary to the first portion of the first single-stranded nucleic acid molecule;
a third single-stranded nucleic acid molecule (D_{A}) comprising a second label of the FRET pair conjugated thereto, the third single-stranded nucleic acid molecule comprising a first portion, a second portion, and a third portion, wherein the second portion of the third single-stranded nucleic acid molecule is complementary to the second portion of the first single-stranded nucleic acid molecule, and wherein the third portion of the third single-stranded nucleic acid molecule is at least partially complementary to the first portion of the second single-stranded nucleic acid molecule; and
a fourth single-stranded nucleic acid molecule (D_{T}) that is at least partially complementary to the first portion of the third single-stranded nucleic acid molecule, wherein the fourth single-stranded nucleic acid molecule comprises a targeting moiety;
contacting the sample with the nucleic acid complex;
measuring an intensity of a signal produced from the contacting of the sample with the nucleic acid complex; and
determining the pH and the K⁺ concentration based on the signal.

Another aspect of the disclosure provides a nucleic acid complex. The nucleic acid complex comprises:
a first single-stranded nucleic acid molecule (D_{K}) comprising a K⁺ fluorophore conjugated to the first single-stranded nucleic acid molecule, the first single-stranded nucleic acid molecule including a first portion and a second portion;
a second single-stranded nucleic acid molecule (D_{D}) comprising a first label of a fluorescence resonance energy transfer (FRET) pair conjugated thereto, the second single-stranded nucleic acid molecule comprising a first portion and a second portion, wherein the second portion of the second single-stranded nucleic acid molecule is complementary to the first portion of the first single-stranded nucleic acid molecule;
a third single-stranded nucleic acid molecule (D_{A}) comprising a second label of the FRET pair conjugated thereto, the third single-stranded nucleic acid molecule comprising a first portion, a second portion, and a third portion, wherein the second portion of the third single-stranded nucleic acid molecule is complementary to the second portion of the first single-stranded nucleic acid molecule, and wherein the third portion of the third single-stranded nucleic acid molecule is at least partially complementary to the first portion of the second single-stranded nucleic acid molecule; and
a fourth single-stranded nucleic acid molecule (D_{T}) that is at least partially complementary to the first portion of the third single-stranded nucleic acid molecule, wherein the fourth single-stranded nucleic acid molecule comprises a targeting moiety.

Another aspect of the disclosure relates to a methods for screening a candidate drug in a model cell or organism. The method comprises delivering the nucleic acid complex of the disclosure to the model cell or organism; contacting the cell or organism with the candidate drug; measuring the intensity of the signal; and determining a pH and a K⁺ concentration from the measured signal. In some embodiments, the model cell or organism is a model for a K+ channel disease.

Another aspect of the disclosure relates to a method for detecting the severity of a disease, the progression of the disease, or the presence of a disease. The method comprises: delivering the nucleic acid complex of the disclosure to a sample; measuring the intensity of the signal; and determining a pH and a K⁺ concentration from the measured signal.

Other aspects of the disclosure will be apparent to those skilled in the art in view of the description that follows.

### BRIEF DESCRIPTION OF THE DRAWINGS

The accompanying drawings are included to provide a further understanding of the systems and methods of the disclosure, and are incorporated in and constitute a part of this specification. The drawings are not necessarily to scale, and sizes of various elements may be distorted for clarity. The drawings illustrate one or more embodiment(s) of the disclosure and together with the description serve to explain the principles and operation of the disclosure.

FIGS. 1A-1D illustrate that pHlicKer is a selective, combination reporter for pH and K⁺.

FIGS. 2A-2W illustrate intracellular calibration of pHlicKer.

FIGS. 3A-3I illustrate pH and K⁺ maps in endocytic organelles.

FIGS. 4A-4E illustrate pH and K+ maps in RE of WT and TWIK2−/−BMDM cells.

FIGS. 5A-5I illustrate pHlicKer reveals Kv11.1 channel activity in TGN.

FIGS. 6A-6L illustrate pHlicKer probes channel activity, trafficking defects and rescue of trafficking.

FIGS. 7A-7E illustrate TAC-Rh fluorescence response is a function of both pH and K⁺.

FIGS. 8A-8G illustrate Targeting modules (T) in organelle-specific pHlicKer variants.

FIGS. 9A-9D illustrate calibration of pHlicKer on beads.

FIGS. 10A-10D illustrate Targetability of 3W^{EE} and 3W^{RE}.

FIGS. 11A-11D illustrate Kv11.1 channel activity in transmembrane ion gradients equivalent to plasma membrane and recycling endosome.

FIG. 12 illustrates spectral characteristics of pHlicKer probe.

FIG. 13 illustrates the UV-Vis absorption spectrum of ssDNA-TAC-Rh.

FIG. 14 illustrates the construction of three-way junction (3WJ).

FIGS. 15A-15D illustrate characterization of pHlicKer^{RE}.

FIGS. 16A-16B illustrate characterization of pHlicKer^{EE/TGN}.

FIGS. 17A-17B illustrate characterization of pHlicKer^{Biotin}.

FIGS. 18A-18D illustrate characterization of 3W^{RE} and 3W^{EE/TGN}.

FIGS. 19A-19B illustrate normalized ratio of fluorescence intensity of donor to that of acceptor (D/A) of pHlicKer^{Biotin} on beads as a function of pH as shown in FIG. 19A, and pHlicKer^{Biotin} K_{d} as a function of pH. Error bars indicate mean±s. e. m. of n=3 experiments as shown in FIG. 19B.

FIG. 20 illustrates immunoblot analysis of lysates isolated from cells expressing G601S-Kv11.1 channels in control conditions (lane 1) or after overnight incubation in dofetilide (lane 2=1 nM, lane 3=10 nM, lane 4=100 nM, lane 5=1 μM, lane 6=10 μM).

### DETAILED DESCRIPTION

Before the disclosed methods and materials are described, it is to be understood that the aspects described herein are not limited to specific embodiments, and as such can, of course, vary. It is also to be understood that the terminology used herein is for the purpose of describing particular aspects only and, unless specifically defined herein, is not intended to be limiting. In view of the present disclosure, the methods and compositions described herein can be configured by the person of ordinary skill in the art to meet the desired need.

Originally, cell surface K⁺ channels are thought to be active only at the plasma membrane. More recently, it has been determined that these channels transit the trans-Golgi network, early and recycling endosomes^{3,4} and that these organelles harbour membrane potentials that can activate voltage-gated K⁺ channels⁵. However, whether cell-surface K⁺ channels are active in organelles is unknown. In this disclosure, the inventors tested the hypothesis that the lumenal K⁺ level within an organelle can reveal voltage-gated K⁺ channel activity in organelles. For example, if cytosolic K⁺ levels exceed that within an organelle, K⁺ is expected to flow down the concentration gradient across the organelle membrane and elevate lumenal K⁺ when a voltage-gated K⁺ channel opens.

Using a pH correctable, ratiometric reporter for K⁺, the inventors were able to probe the compartment-specific activity of a prototypical voltage-gated K⁺ channel, Kv11.1, and show this cell surface channel is active in organelles. Lumenal K⁺ in organelles increased in cells expressing wild-type Kv11.1 channels but not when cells were treated with blockers of Kv11.1 current. In cells expressing mutant Kv11.1 channels, whose transport to the cell surface is impaired, K⁺ levels did not increase in recycling endosomes but did so when the impairment was pharmacologically corrected. By providing a way to map the organelle-specific activity of K⁺ channels, the presently disclosed potassium ion reporting technology could help identify new organellar K⁺ channels or channel modulators with nuanced functions as well as facilitate development of drug treatments for medical conditions resulting from impaired potassium ions channels.

Every fluorescent K⁺ probe works by coordinating K⁺ through sp³ N atoms, where the nitrogen lone pair quenches the fluorophore due to photoinduced electron transfer (PeT)⁸⁻¹⁰. Acidic pH mimics K⁺ binding as protonation of the lone pair causes probes to turn on by alleviating PeT, also weakening probe affinity (K_{d}) for K⁺. Further, organelle pH is variable and its effect on membrane potential or lumenal K⁺ are difficult to predict. Thus, it is non-trivial to deconvolute the contribution of K⁺ from the readout of any fluorescent K⁺ indicator in organelles. The only measure of K⁺ in organelles so far is obtained using electron probe X-ray microanalysis where phagosomal K⁺ in neutrophils was reported to lie between 200-300 mM¹¹.

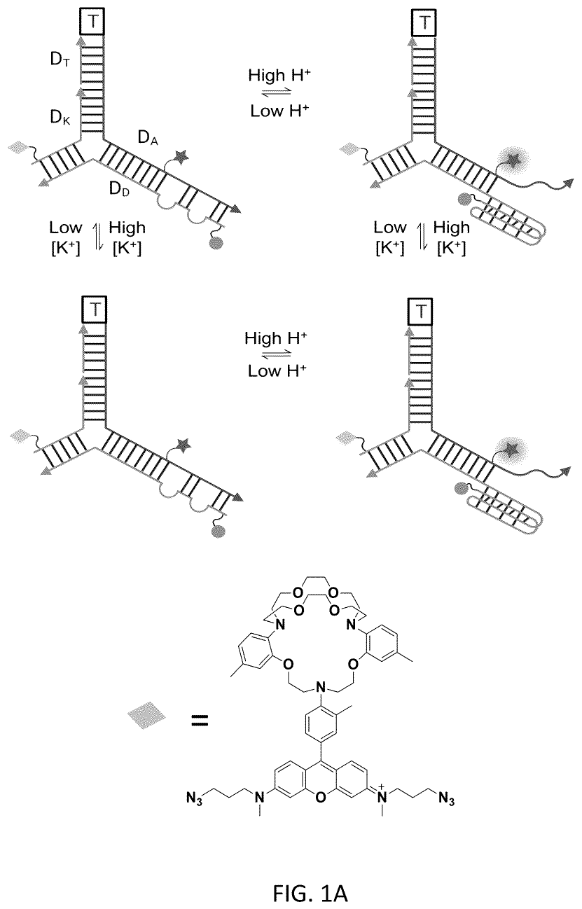

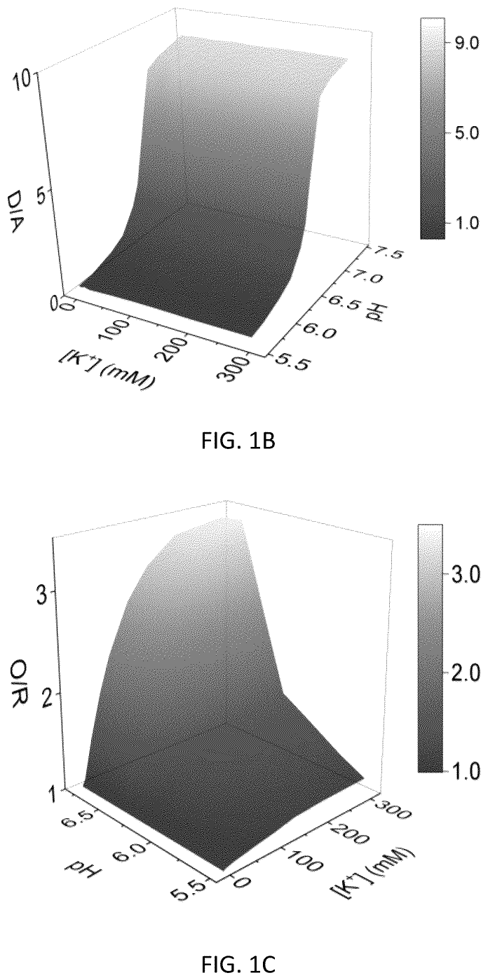

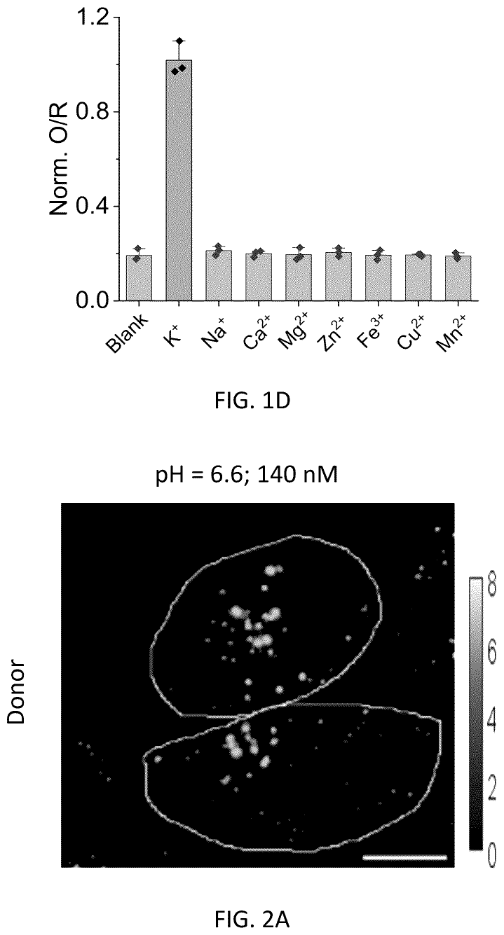

The inventors had developed a DNA-based pH-correctable, intracellular K⁺ reporter, denoted pHlicKer, that can simultaneously report organellar pH and K⁺ with single-organelle addressability. The pHlicKer probe was constructed using DNA because it is biocompatible, suitable for quantitative imaging and one can integrate multiple functions in precise stoichiometries into a single assembly^{12,13,14}. Specifically, pHlicKer is a three-way DNA junction comprising four strands. FIG. 1A illustrates a representative pHlicKer probe which includes a 25-mer oligonucleotide conjugated to a K⁺ indicator at its 5′ end (D_{K}); a 71-mer strand bearing a reference dye (D_{A}), a 57-mer strand bearing a pH sensing module (D_{D}) and a strand harboring an organelle-targeting motif (D_{T}). The quaternary complex displays a targeting module (T) that engages a distinct, cognate cell surface receptor that traffics the probe to the relevant organelle^{15,16}.

In one aspect, the present disclosure relates to a method for simultaneously determining a pH and a K⁺ concentration in a sample. The method comprises providing a nucleic acid complex; contacting the sample with the nucleic acid complex; measuring an intensity of a signal produced from the contacting of the sample with the nucleic acid complex; and determining the pH and the K⁺ concentration based on the signal.

In various embodiments, the nucleic acid complex comprises a first single-stranded nucleic acid molecule (D_{K}) comprising a K⁺ fluorophore conjugated to the first single-stranded nucleic acid molecule, the first single-stranded nucleic acid molecule including a first portion and a second portion; a second single-stranded nucleic acid molecule (D_{D}) comprising a first label of a FRET pair conjugated thereto, the second single-stranded nucleic acid molecule comprising a first portion and a second portion, wherein the second portion of the second single-stranded nucleic acid molecule is complementary to the first portion of the first single-stranded nucleic acid molecule; a third single-stranded nucleic acid molecule (D_{A}) comprising a second label of the FRET pair conjugated thereto, the third single-stranded nucleic acid molecule comprising a first portion, a second portion, and a third portion, wherein the second portion of the third single-stranded nucleic acid molecule is complementary to the second portion of the first single-stranded nucleic acid molecule, and wherein the third portion of the third single-stranded nucleic acid molecule is at least partially complementary to the first portion of the second single-stranded nucleic acid molecule; and a fourth single-stranded nucleic acid molecule (D_{T}) that is at least partially complementary to the first portion of the third single-stranded nucleic acid molecule, wherein the fourth single-stranded nucleic acid molecule comprises a targeting moiety.

In various embodiments, the K⁺ fluorophore may comprise a triazacryptand K(+)-selective ionophore. In various embodiments as otherwise described herein, the triazacryptand K(+)-selective ionophore may be coupled to rhodamine.

In various embodiments, the K⁺ fluorophore may be coupled to the 5′-end of the first single-stranded nucleic acid molecule.

In various embodiments, the K⁺ fluorophore comprises a formula of:

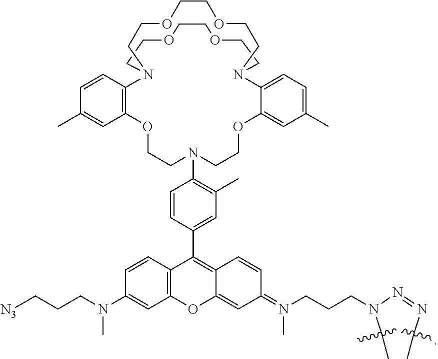

In various embodiments, the K⁺ fluorophore comprises a formula of:

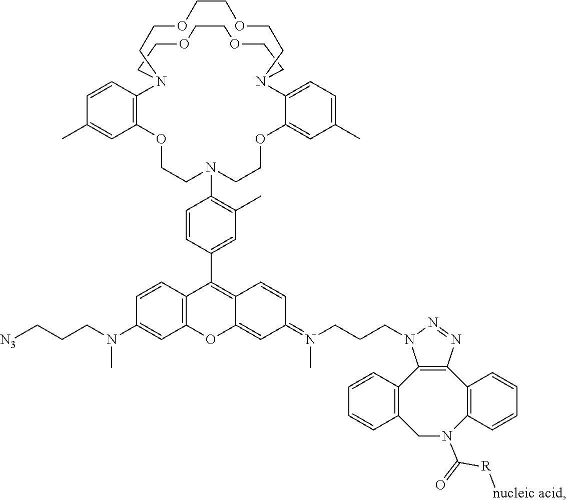

wherein R is a linker.

In various embodiments, the FRET pair may be Alexa 647/Alexa 488.

In various embodiments, the intensity of the signal dependent on change in pH may vary as a function of the conformation of the nucleic acid complex. In various embodiments as otherwise described herein, the intensity of the signal may vary as a function of at least one of a distance between the first label and the second label of the FRET pair and a relative orientation of the first label and the second label of the FRET pair.

In various embodiments, the second single-stranded nucleic acid molecule and the third single-stranded nucleic acid molecule may form an i-motif under acidic conditions.

In various embodiments, the second single-stranded nucleic acid molecule may be capable of forming an intramolecular complex comprising two parallel-stranded C.CH+ base paired duplexes that are intercalated in an anti-parallel orientation under acidic conditions.

In various embodiments, the targeting moiety may target a K⁺ cell surface channel, a K⁺ cellular organelle channel, or a K⁺ transporter.

In various embodiments, the targeting moiety may comprise a TfR aptamer, MSR1 receptor, or a scFv-furin.

In various embodiments, the first, the second, the third, or the fourth single-stranded nucleic acid molecule may be less than 200 nucleotides, or less than 100nucleotides, or less than 50 nucleotides.

In various embodiments, the determined K⁺ concentration may be in a range of 0.1 mM to 1 mM, or 1 mM to 10 mM, or 10 mM to 100 mM, or 100 mM to 300 mM.

In various embodiments, the determined pH may be in a range of 5.8 to 7.0.

In another aspect, the present disclosure relates to a nucleic acid complex. The nucleic acid complex comprises a first single-stranded nucleic acid molecule (D_{K}) comprising a K⁺ fluorophore conjugated to the first single-stranded nucleic acid molecule, the first single-stranded nucleic acid molecule including a first portion and a second portion; a second single-stranded nucleic acid molecule (D_{D}) comprising a first label of a FRET pair conjugated thereto, the second single-stranded nucleic acid molecule comprising a first portion and a second portion, wherein the second portion of the second single-stranded nucleic acid molecule is complementary to the first portion of the first single-stranded nucleic acid molecule; a third single-stranded nucleic acid molecule (D_{A}) comprising a second label of the FRET pair conjugated thereto, the third single-stranded nucleic acid molecule comprising a first portion, a second portion, and a third portion, wherein the second portion of the third single-stranded nucleic acid molecule is complementary to the second portion of the first single-stranded nucleic acid molecule, and wherein the third portion of the third single-stranded nucleic acid molecule is at least partially complementary to the first portion of the second single-stranded nucleic acid molecule; and a fourth single-stranded nucleic acid molecule (D_{T}) that is at least partially complementary to the first portion of the third single-stranded nucleic acid molecule, wherein the fourth single-stranded nucleic acid molecule comprises a targeting moiety.

In various embodiments, the nucleic acid complex may be pHlicKer^{RE} comprising a first nucleic acid strand (D_{K}) having a sequence of 5′-DBCO-TEG-ATCAAGGTGGCGAGAGCGACGATCC-3′ [SEQ ID NO:1]; a second nucleic acid strand (DD) having a sequence of 5′-Alexa-488-CCCCTAACCCCTAACCCCTAACCCCATATATAGGTCAACTCTTCTCGCCACCTTGA T-3′ [SEQ ID NO:2]; a third nucleic acid strand (D_{A}^{RE}) having a sequence of 5′-CACTGCACACCAGACAGCAAGGATCGTCGCAGAGTTGACCT (Alexa647N) ATATATT TTGTTATGTGTTATGTGTTAT-3′ [SEQ ID NO:3]; and a fourth nucleic acid strand (D_{T}^{RE}) having a sequence of 5′-TTGCTGTCTGGTGTGCAGTGTTGATGGGGGAUCAAUCCAAGGGACCCGGAAACG CUCCCUUACACCCC-3′ [SEQ ID NO:4]. As described herein, DBCO-TEG represents dibenzocyclooctyne triethylene glycol.

In various embodiments, the nucleic acid complex may be pHlicKer^{EE/TGN} comprising a first nucleic acid strand (D_{K}) having a sequence of 5′-DBCO-TEG-ATCAAGGTGGCGAGAGCGACGATCC-3′ [SEQ ID NO:1]; a second nucleic acid strand (DD) having a sequence of 5′-Alexa-488-CCCCTAACCCCTAACCCCTAACCCCATATATAGGTCAACTCTTCTCGCCACCTTGA T-3′ [SEQ ID NO:2]; a third nucleic acid strand (D_{A}^{EE/TGN}) having a sequence of 5′-ATATATATACACCAGACAGCAAGGATCGTCGCAGAGTTGACCT (Alexa647N) ATATT TTGTTATGTGTTATGTGTTAT-3′ [SEQ ID NO:6]; and a fourth nucleic acid strand (D_{T}^{EE/TGN}) having a sequence of 5′-TTGCTGTCTGGTGTATATATAT-3′ [SEQ ID NO:5].

In various embodiments, the nucleic acid complex may be pHlicKer^{Biotin} comprising a first nucleic acid strand (D_{K}) having a sequence of 5′-DBCO-TEG-ATCAAGGTGGCGAGAGCGACGATCC-3′ [SEQ ID NO:1]; a second nucleic acid strand (DD) having a sequence of 5′-Alexa-488-CCCCTAACCCCTAACCCCTAACCCCATATATAGGTCAACTCTTCTCGCCACCTTGA T-3′ [SEQ ID NO:2]; a third nucleic acid strand (D_{A}^{RE}) having a sequence of 5′-CACTGCACACCAGACAGCAAGGATCGTCGCAGAGTTGACCT (Alexa647N) ATATATT TTGTTATGTGTTATGTGTTAT-3′ [SEQ ID NO:3]; and a fourth nucleic acid strand (D_{T}^{Biotin}) having a sequence of 5′-GCGACGATCCTTGCTGTCTGGTGTGCAGTG/3BioTEG/-3′ [SEQ ID NO:7]. As described herein, 3BioTEG represents 3′-Biotin-TEG, where TEG represents triethylene glycol.

In various embodiments, the nucleic acid complex may be pHlicKer^{EE} comprising a first nucleic acid strand (D_{K}) having a sequence of 5′-DBCO-TEG-ATCAAGGTGGCGAGAGCGACGATCC-3′ [SEQ ID NO:1]; a second nucleic acid strand (D_{D}) having a sequence of 5′-Alexa-488-CCCCTAACCCCTAACCCCTAACCCCATATATAGGTCAACTCTTCTCGCCACCTTGA T-3′ [SEQ ID NO:2]; a third nucleic acid strand (D_{A}^{EE}) having a sequence of 5′-ATATATATACACCAGACAGCAAGGATCGTCGCAGAGTTGACCT (Alexa647N) ATATTTTGTTATGTGTTATGTGTTAT-3′ [SEQ ID NO:6]; and a fourth nucleic acid strand (D_{T}^{EE}) having a sequence of 5′-TTGCTGTCTGGTGTATATATAT-3′ [SEQ ID NO:5].

In various embodiments, the nucleic acid complex may be pHlicKer^{TGN} comprising a first nucleic acid strand (D_{K}) having a sequence of 5′-DBCO-TEG-ATCAAGGTGGCGAGAGCGACGATCC-3′ [SEQ ID NO:1]; a second nucleic acid strand (D_{D}) having a sequence of 5′-Alexa-488-CCCCTAACCCCTAACCCCTAACCCCATATATAGGTCAACTCTTCTCGCCACCTTGA T-3′ [SEQ ID NO:2]; a third nucleic acid strand (D_{A}^{TGN}) having a sequence of 5′-ATATATATACACCAGACAGCAAGGATCGTCGCAGAGTTGACCT (Alexa647N) ATATTTTGTTATGTGTTATGTGTTAT-3′ [SEQ ID NO:6]; and a fourth nucleic acid strand (D_{T}^{TGN}) having a sequence of 5′-TTGCTGTCTGGTGTATATATAT-3′ [SEQ ID NO:5].

In various embodiments, the nucleic acid complex may be 3WJ comprising a first nucleic acid strand (2) having a sequence of 5′-TTGCTGTCTGGTGTGCAGTGTTGAT-3′ [SEQ ID NO:9]; a second nucleic acid strand (4) having a sequence of 5′-CCCCTAACCCCTAACCCCTAACCCCATATATAGGTCAACTCTTCTCGCCACCTTGA T-3′ [SEQ ID NO:11]; a third nucleic acid strand (3) having a sequence of 5′-CACTGCACACCAGACAGCAAGGATCGTCGCAGAGTTGACCTATATATTTTGTTATG TGTTATGTGTTAT-3′ [SEQ ID NO:10]; and a fourth nucleic acid strand (1) having a sequence of 5′-ATCAAGGTGGCGAGAGCGACGATCC-3′ [SEQ ID NO:8].

In various embodiments, the nucleic acid complex may be 3W^{RE} comprising a first nucleic acid strand (1) having a sequence of 5′-ATCAAGGTGGCGAGAGCGACGATCC-3′ [SEQ ID NO:8]; a second nucleic acid strand (4) having a sequence of 5′-CCCCTAACCCCTAACCCCTAACCCCATATATAGGTCAACTCTTCTCGCCACCTTGA T-3′ [SEQ ID NO:11]; a third nucleic acid strand (D_{A}^{RE}) having a sequence of 5′-CACTGCACACCAGACAGCAAGGATCGTCGCAGAGTTGACCT (Alexa647N) ATATATT TTGTTATGTGTTATGTGTTAT-3′ [SEQ ID NO:3]; and a fourth nucleic acid strand (D_{T}^{RE}) having a sequence of 5′-TTGCTGTCTGGTGTGCAGTGTTGATGGGGGAUCAAUCCAAGGGACCCGGAAACG CUCCCUUACACCCC-3′ [SEQ ID NO:4].

In various embodiments, the nucleic acid complex may be 3W^{EE/TGN} comprising a first nucleic acid strand (1) having a sequence of 5′-ATCAAGGTGGCGAGAGCGACGATCC-3′ [SEQ ID NO:8]; a second nucleic acid strand (4) having a sequence of 5′-CCCCTAACCCCTAACCCCTAACCCCATATATAGGTCAACTCTTCTCGCCACCTTGA T-3′ [SEQ ID NO:11]; a third nucleic acid strand (D_{A}^{EE/TGN}) having a sequence of 5′-ATATATATACACCAGACAGCAAGGATCGTCGCAGAGTTGACCT (Alexa647N) ATATTTTGTTATGTGTTATGTGTTAT-3′ [SEQ ID NO:6]; and a fourth nucleic acid strand (D_{T}^{EE/TGN}) having a sequence of 5′-TTGCTGTCTGGTGTATATATAT-3′ [SEQ ID NO:5].

FIGS. 1A-1D illustrate that pHlicKer is a selective, combination reporter for pH and K⁺. FIG. 1A shows working principle of pHlicKer. D_{K}, D_{D} and D_{A} represent three DNA strands that display a targeting module, T, on pHlicKer. pH-induced FRET between Alexa Fluor 488 (donor; green sphere) and Alexa Fluor 647 (acceptor; magenta star) reports on pH ratiometrically. K⁺-sensitive dye, TAC-Rh (yellow diamond; λₑₓ=560 nm), and Alexa Fluor 647 (λₑₓ=650 nm) report K⁺ ratiometrically by direct excitation. FIGS. 1B-1C show 3D surface plot of D/A, the pH response (FIG. 1B), and O/R, the K⁺ response (FIG. 1C), of pHlicKer^{Biotin} immobilized on beads as a function of pH and [K⁺]. The pH response of pHlicKer is insensitive to K⁺, but its K⁺ response is sensitive to acidic pH. FIG. 1D shows normalized O/R values of pHlicKer^{Biotin} in response to other biologically relevant cations show pHlicKer is specific to K⁺. Concentrations were: K⁺ and Na⁺ (150 mM); Ca²⁺ and Mg²⁺ (1.5 mM); Zn²⁺, Fe³⁺, Cu²⁺ and Mn²⁺ (100 μM). Error bars represent standard error of mean (s.e.m.) from three independent experiments.

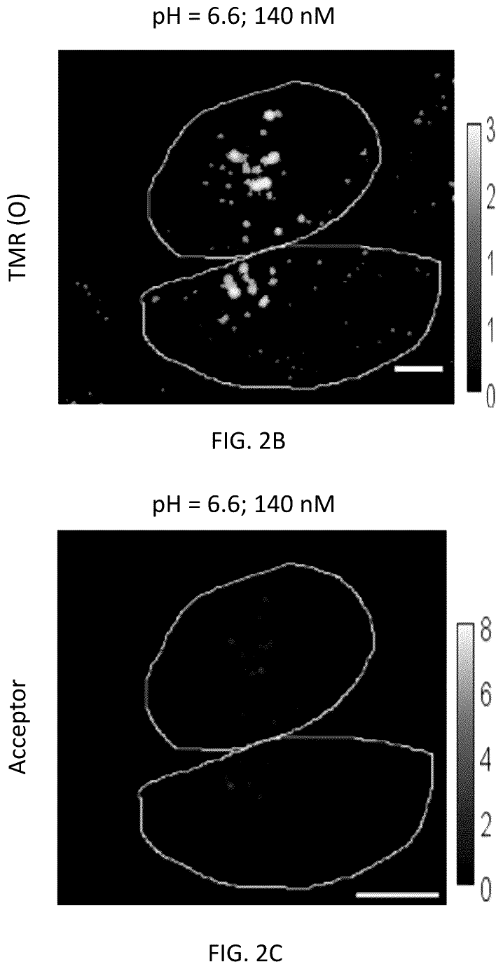

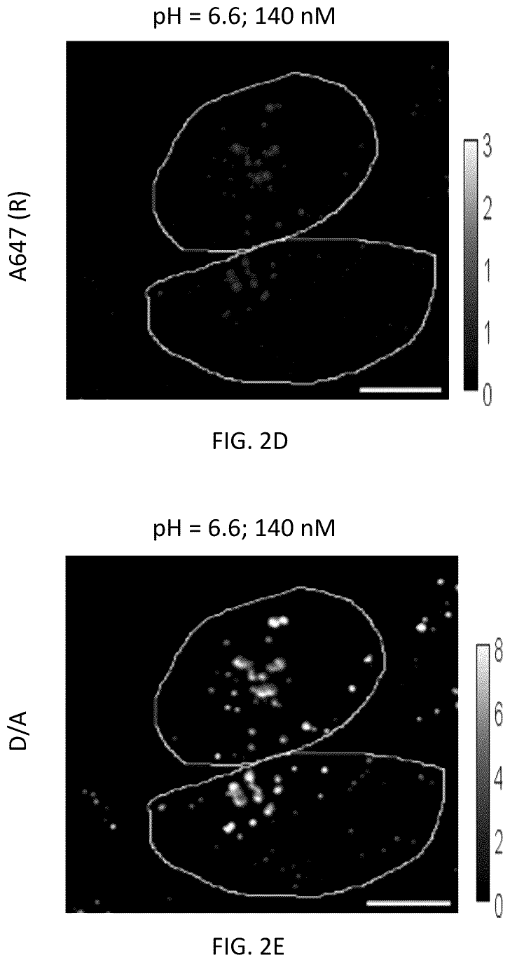

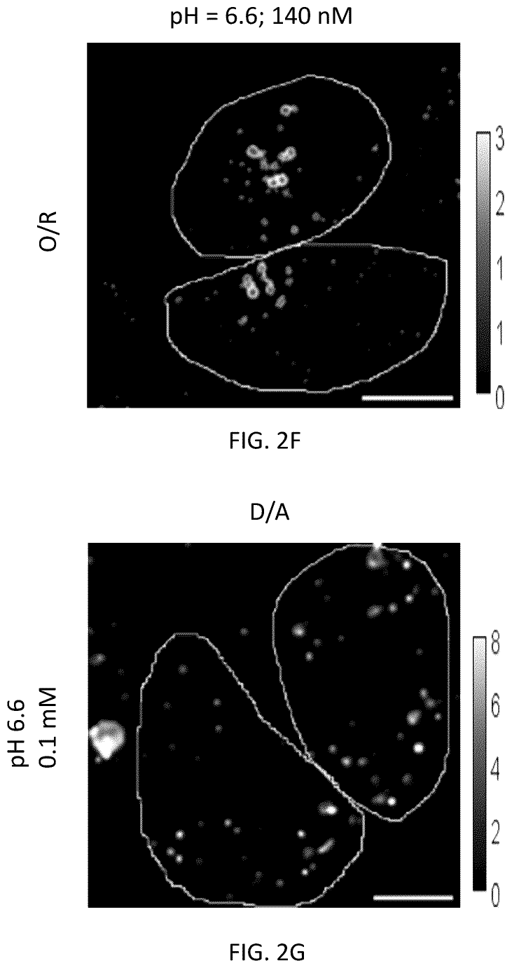

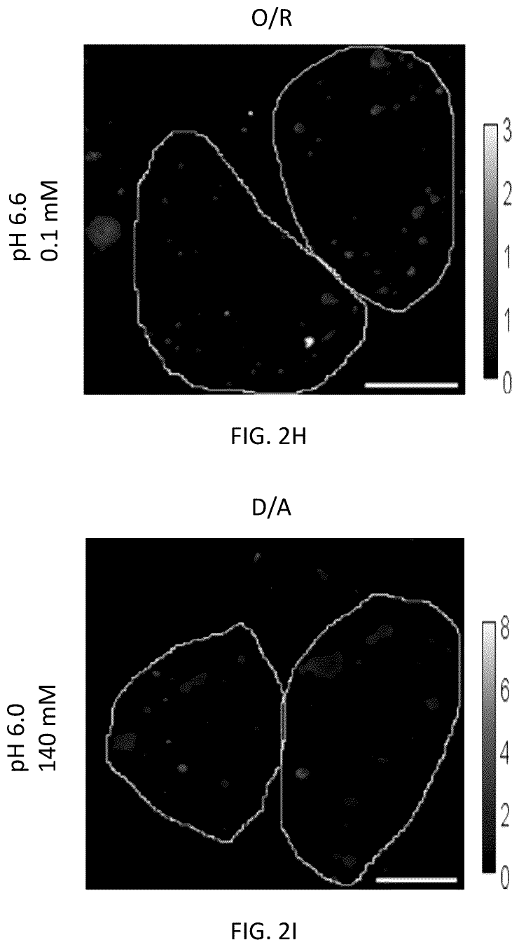

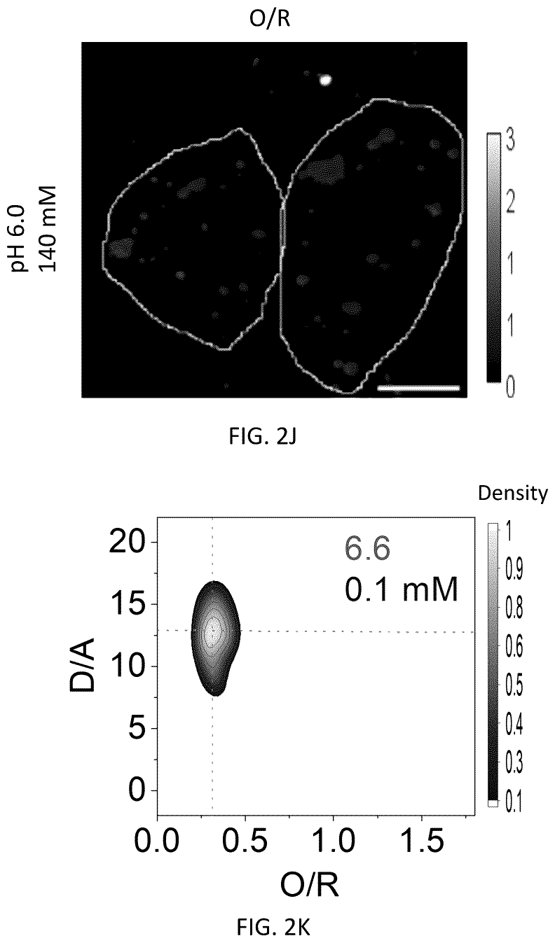

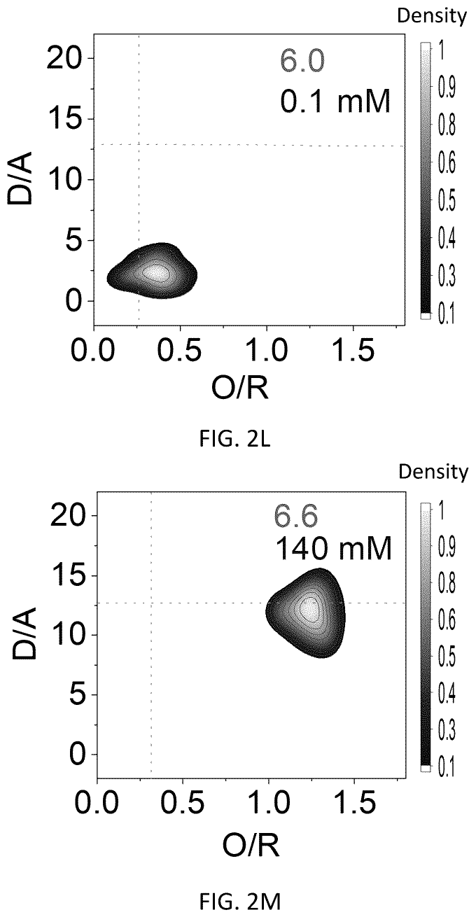

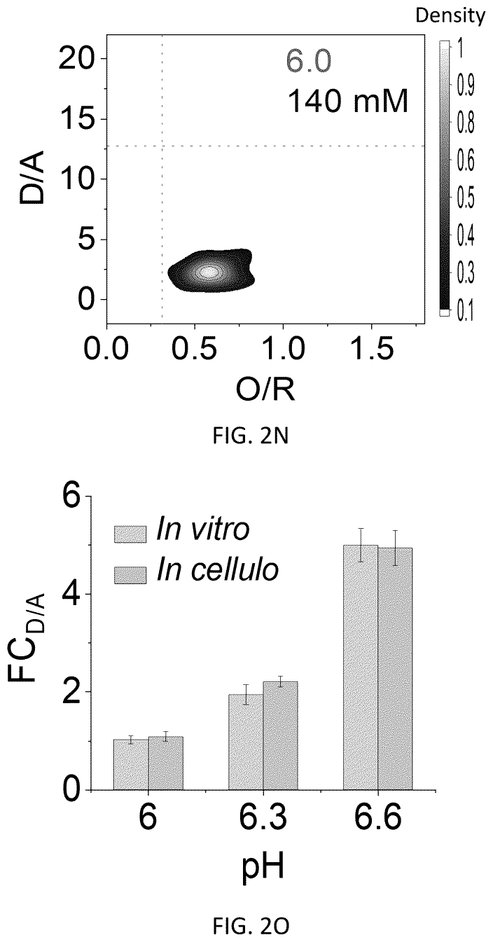

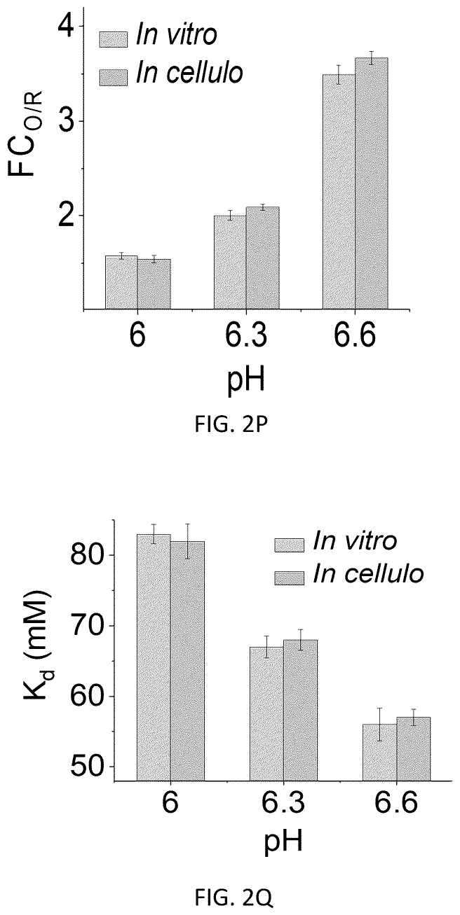

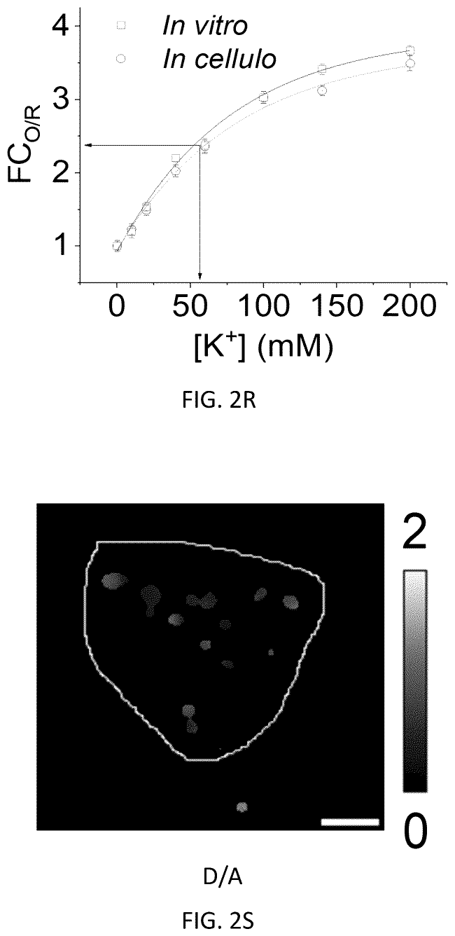

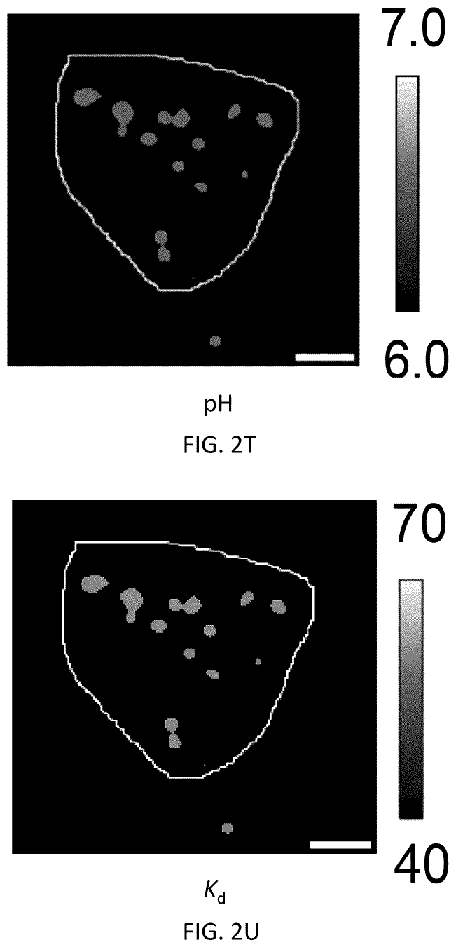

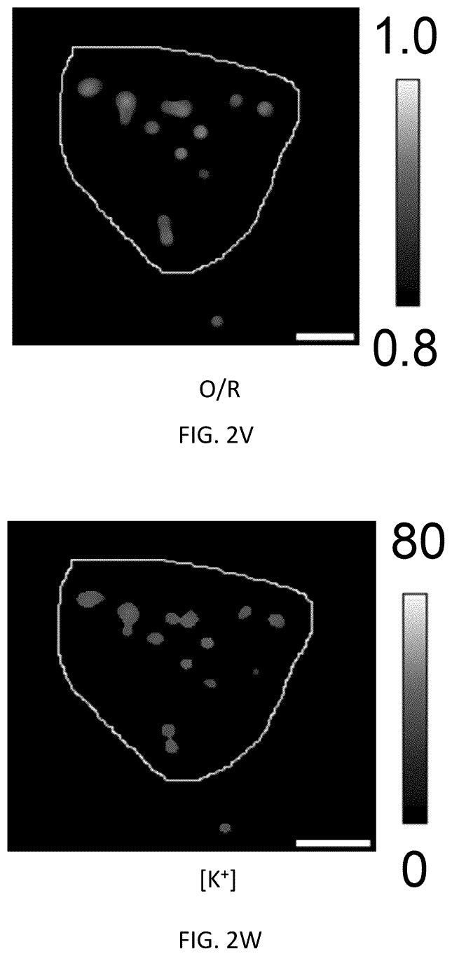

FIGS. 2A-2W illustrate intracellular calibration of pHlicKer. FIGS. 2A-2D show representative pHlicKer^{RE}-labeled RE of HEK 293T cells imaged in the donor (FIG. 2D), TAC-Rh (TMR, O), acceptor (FIG. 2A), and Alexa Fluor 647 (R) channels, clamped at pH 6.6 and 140 mM K⁺. FIGS. 2E and 2F show the corresponding pixel-wise pseudocolor images. FIGS. 2G-2J show representative pseudo-coloured D/A and O/R maps of RE in HEK 293T cells clamped at the indicated pH and [K⁺]. FIGS. 2K-2N show 2-IM profiles of HEK 293T cells clamped at the indicated pH (red font) and [K⁺] (blue font). Dotted lines are a frame of reference centered on the maximum in FIG. 2K. Experiments were performed in triplicate (n=10 cells, n=100 endosomes). FIGS. 2O-2P show in vitro and in cellulo response of pHlicKer given by fold change (FC) of (FIG. 2O) D/A from pH 6.0-6.6 at 0.1 mM [K⁺] and (FIG. 2P) O/R ratios from 0.1-140 mM [K⁺] at the indicated pH. FIG. 2Q shows in vitro and in cellulo K_{d} values of pHlicKer determined at the indicated pH. FIG. 2R shows O/R values of pHlicKer at pH 6.6 as a function of [K⁺] obtained in vitro and in cellulo. FIGS. 2S-2W show [K⁺] maps are obtained by converting the (FIG. 2S) D/A map of pHlicKer^{RE}-labeled REs into (FIG. 2T) a pH map. This is converted into (FIG. 2U) a K_{d} map, where pH at every pixel is replaced by the value of K_{d} at that pixel. The product of the (FIG. 2V) O/R map and K_{d} map yields (FIG. 2W) the [K⁺] map. “f(x)” is function of x. Data are from one experiment representative of three independent experiments and are presented as mean±s.e.m in FIGS. 2O-2R. All scale bars, 5 μm.

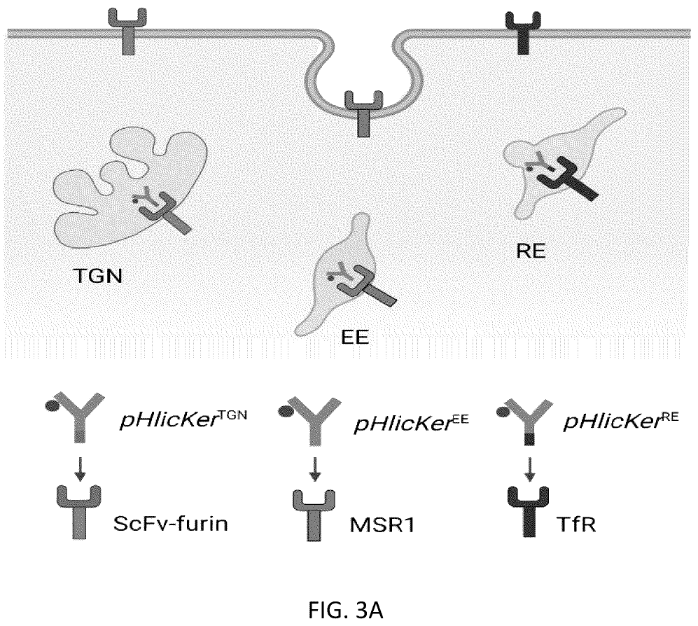

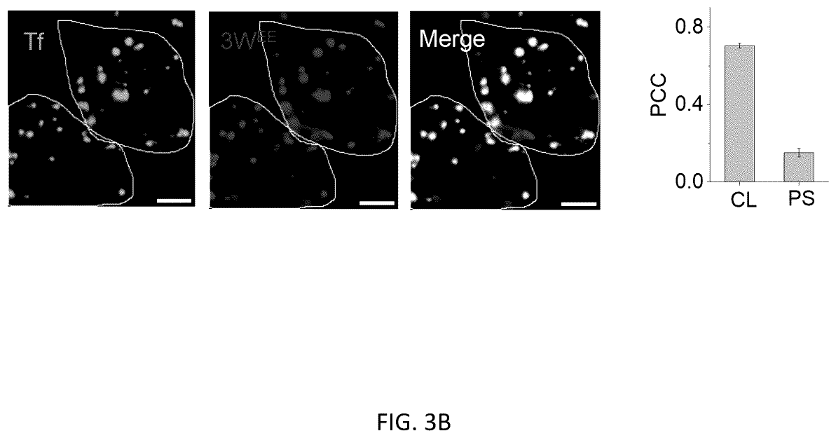

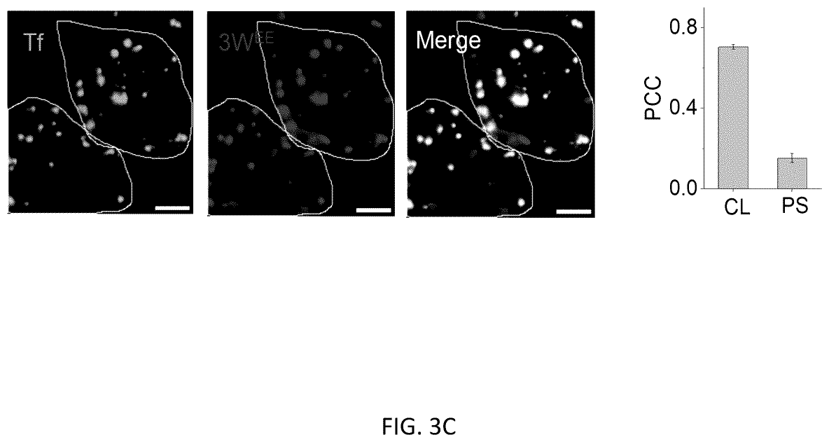

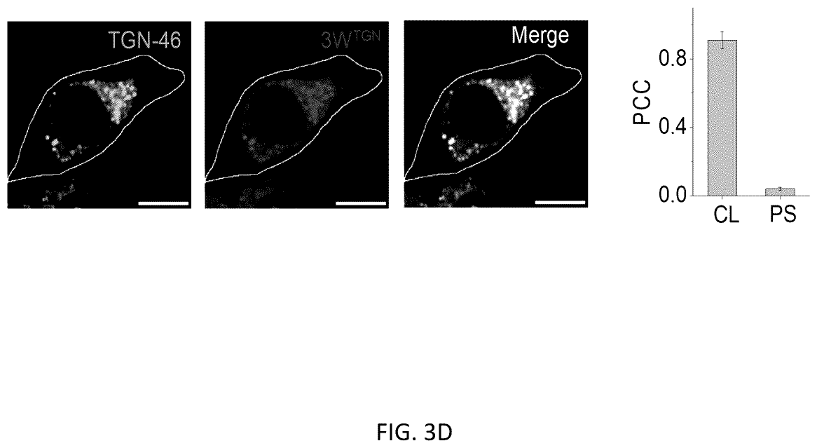

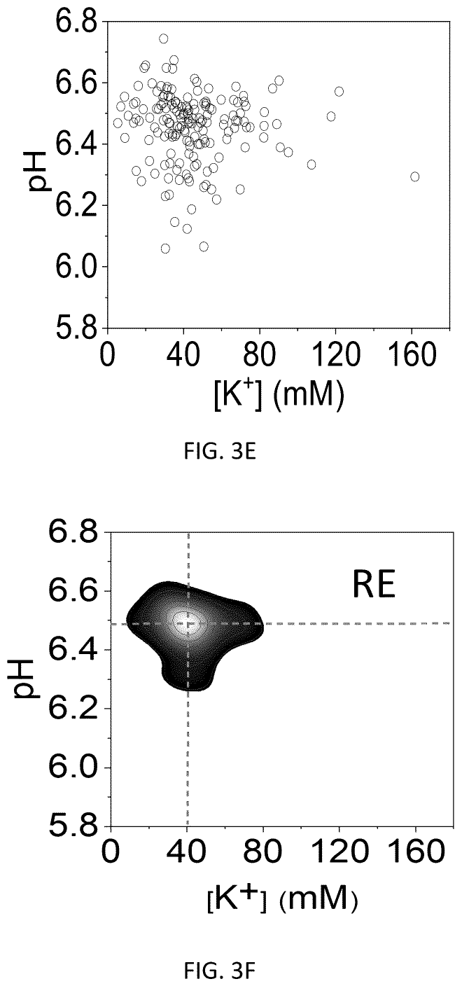

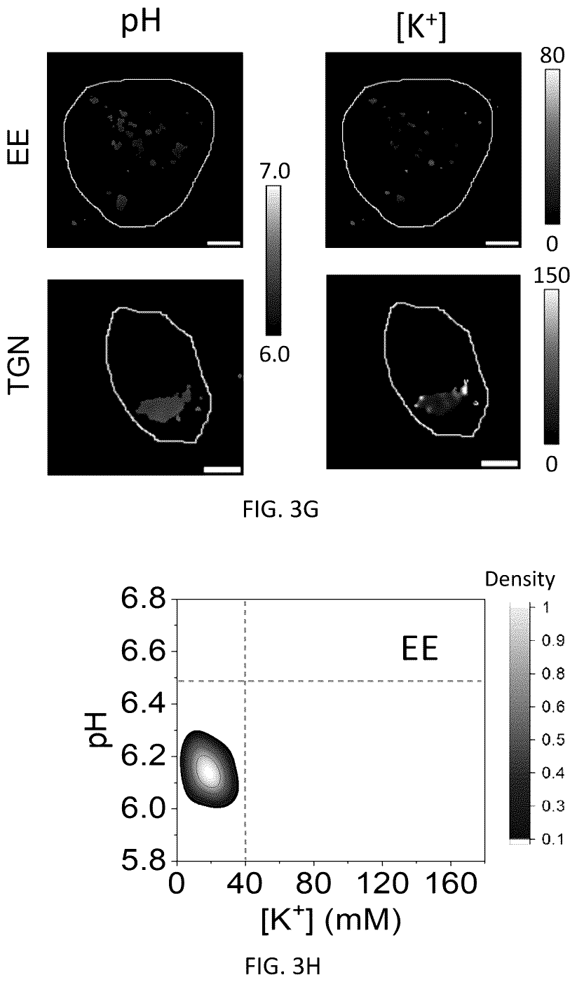

FIGS. 3A-3I illustrate pH and K⁺ maps in endocytic organelles. FIG. 3A shows a schematic of organelle targeting: pHlicKer^{RE} targets the transferrin receptor (TfR) mediated endocytosis, displays a TfR aptamer and localizes in REs. pHlicKer^{EE} targets scavenger receptor (MSR1)-mediated endocytosis, leverages its dsDNA domains and marks EEs. pHlicKer^{TGN} targets retrograde trafficking of furin, displays a d(AT)₄ domain that binds an scFv domain on furin to mark the TGN. Cells express each receptor endogenously or by transfection. FIG. 3B shows colocalization of fluorescently labeled Tf (Tf-Alexa488, cyan) with EE labeled by 3W^{EE} (magenta) in hMSR1-transfected HEK 293T cells. FIG. 3C shows colocalization between RE tracer (transferrin-Alexa488, cyan) and 3W^{RE} (magenta) in HEK 293T cells. FIG. 3D shows colocalization between TGN marker (TGN46-mCherry, cyan) and 3W^{TGN} (magenta) in scFv-furin-transfected HEK 293T cells. Scale bar, 5 μm. Pearson's correlation coefficient (PCC) of colocalization (CL) and pixel shift (PS) for FIGS. 3B-3D is shown on the right. Error bars represent mean±s.e.m. of three independent trials for n=15 cells. FIG. 3E shows representative scatter plots of pH versus [K⁺] of RE in HEK 293T cells. Each data point corresponds to a single endosome. FIG. 3G shows representative pseudocolor pH and [K⁺] maps of EE and TGN labeled with pHlicKer^{EE/TGN}. FIGS. 3F, 3H-3I show 2-IM profiles of pH and [K⁺] in RE, EE, and TGN. Data represents one out of three independent experiments (n=10 cells, n=100 endosomes).

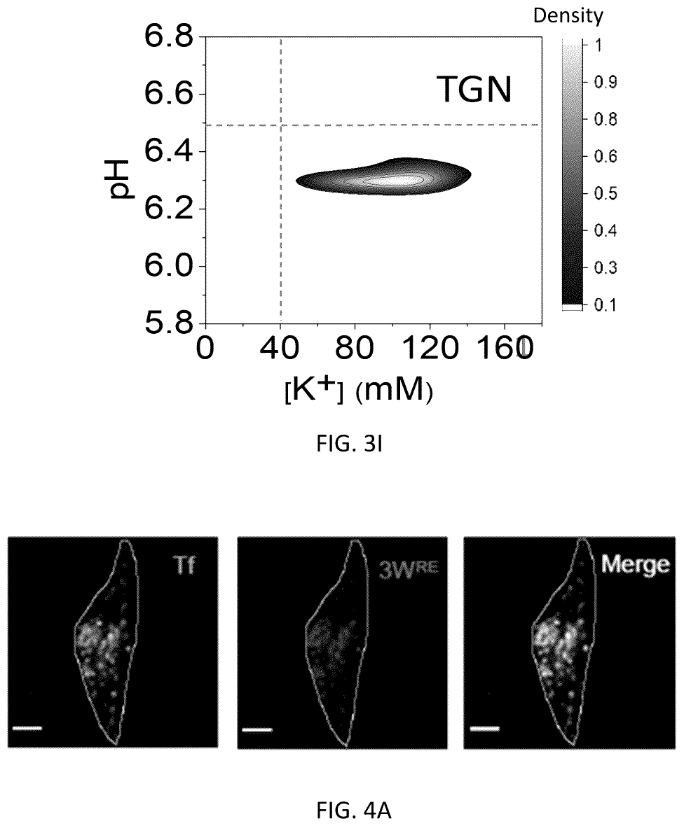

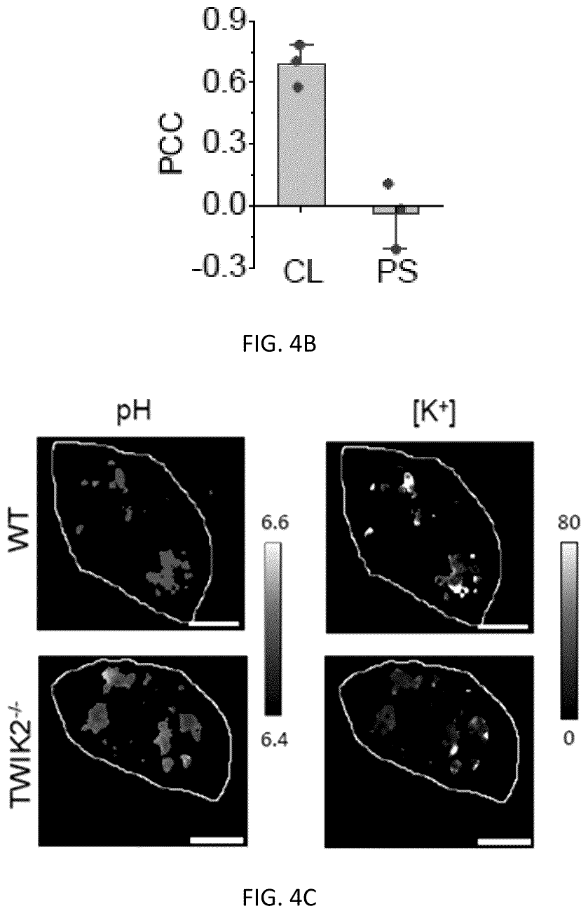

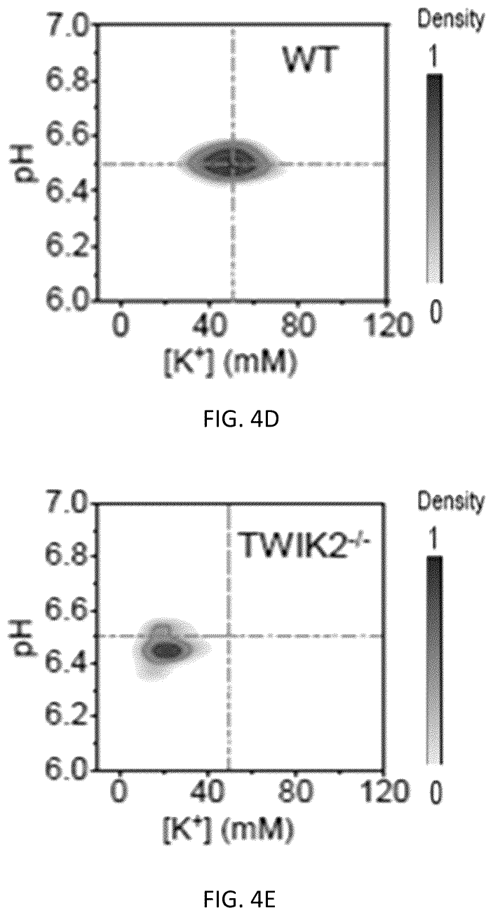

FIGS. 4A-4E illustrate pH and K+ maps in RE of WT and TWIK2−/−BMDM cells. FIG. 4A shows colocalization between RE tracer (Tf-Alexa488, cyan) and 3W^{RE} (magenta) in BMDM cells. FIG. 4B shows PCC of colocalization (CL) and pixel shift (PS) for FIG. 4A. Error bars represent mean±s.e.m. of three independent trials for n=16 cells. FIG. 4C shows representative pseudocolor pH and [K+] maps of RE in WT and TWIK2−/− of BMDM cells labeled with pHlicKerRE. FIGS. 4D-4E show 2-IM profiles of pH and [K+] of RE in WT and TWIK2−/− of BMDM cells (FIG. 4D: n=13 cells, n=108 endosomes; FIG. 4E: n=13 cells, n=106 endosomes). Data represent one out of three independent experiments.

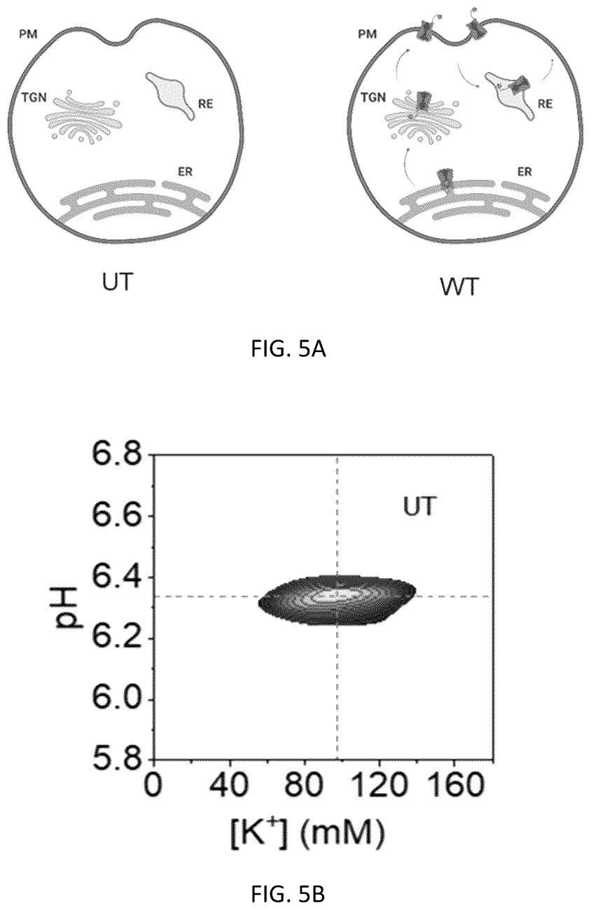

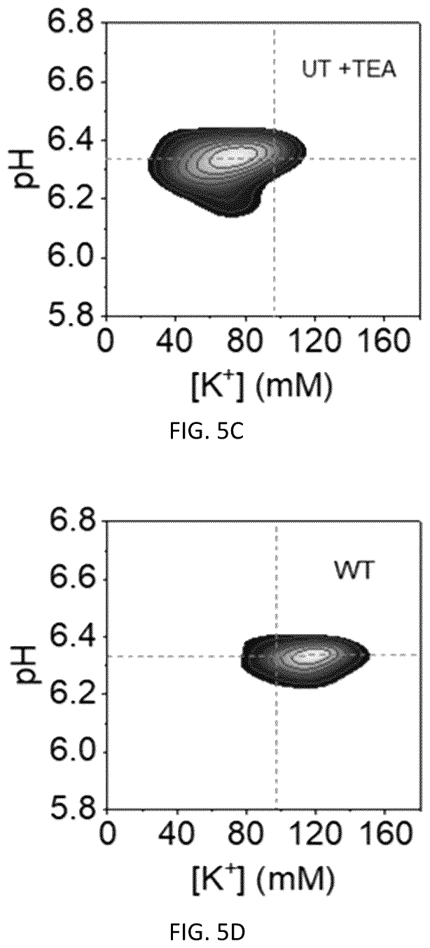

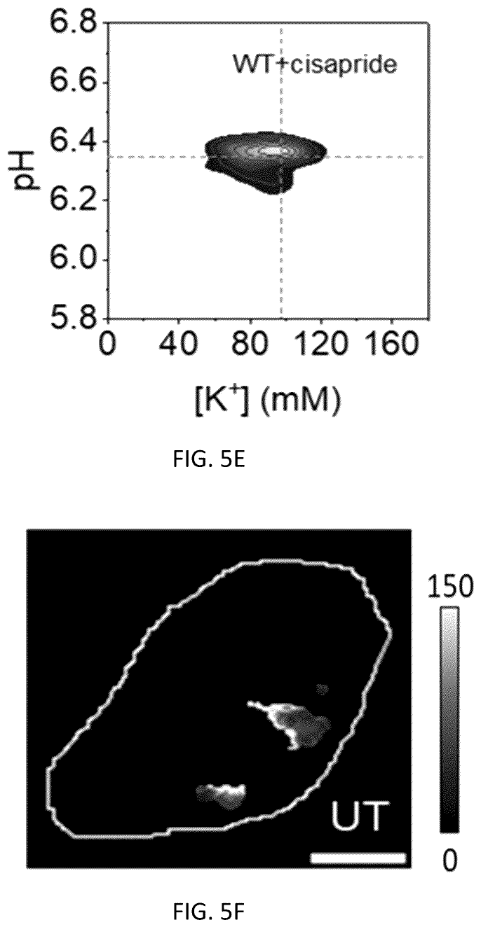

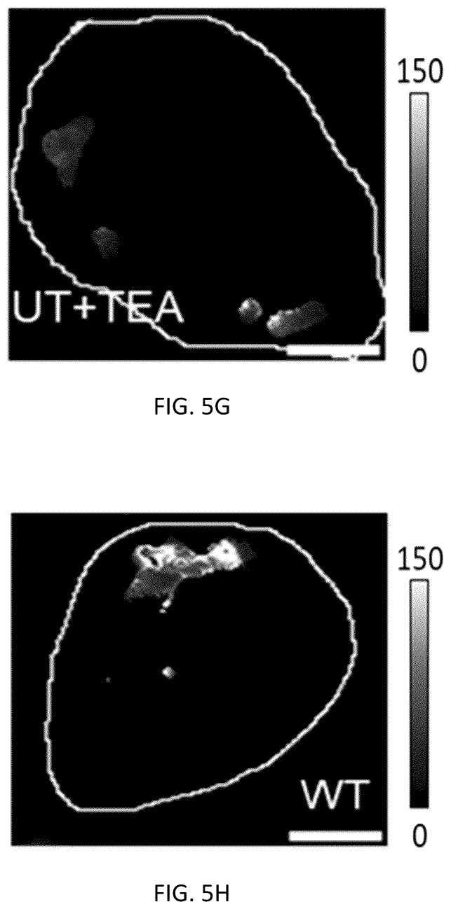

FIGS. 5A-5I illustrate pHlicKer reveals Kv11.1 channel activity in TGN. FIG. 5A shows a schematic of HEK 293T cells without endogenous Kv11.1 channels (UT) and stably expressing wild type Kv11.1 channels (WT). Kv11.1 channels traffic to the plasma membrane (PM) of WT cells via the TGN and are recycled via REs. FIGS. 5B-5E show [K⁺]_{TGN} and pH values in UT cells without (FIG. 5B) and treated with tetraethyl ammonium chloride (TEA) (FIG. 5C), WT cells without (FIG. 5D) and treated with cisapride (FIG. 5E). FIGS. 5F-5I show representative pseudocolor [K⁺] maps of TGN labeled with pHlicKer^{TGN} for the indicated conditions. Data are from one representative trial of independent experiments in triplicate (FIGS. 5B-5E).

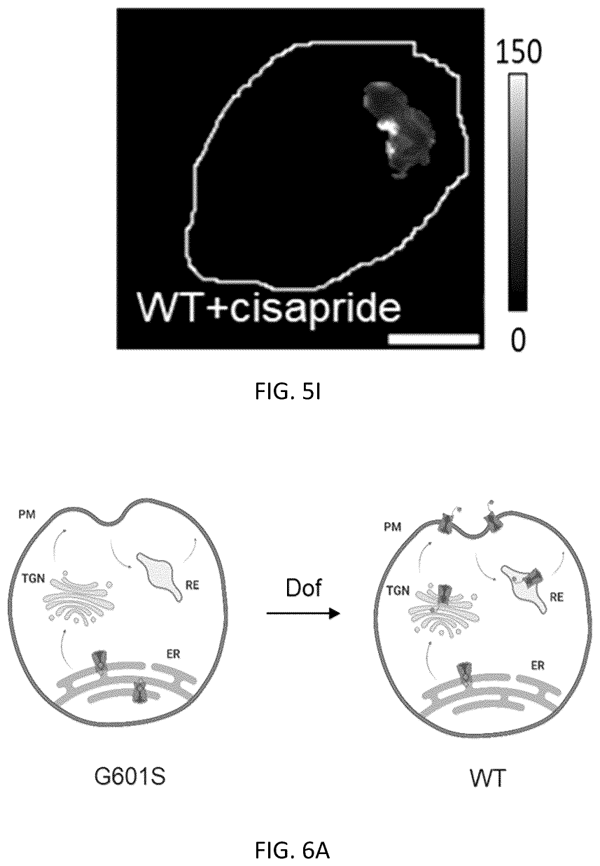

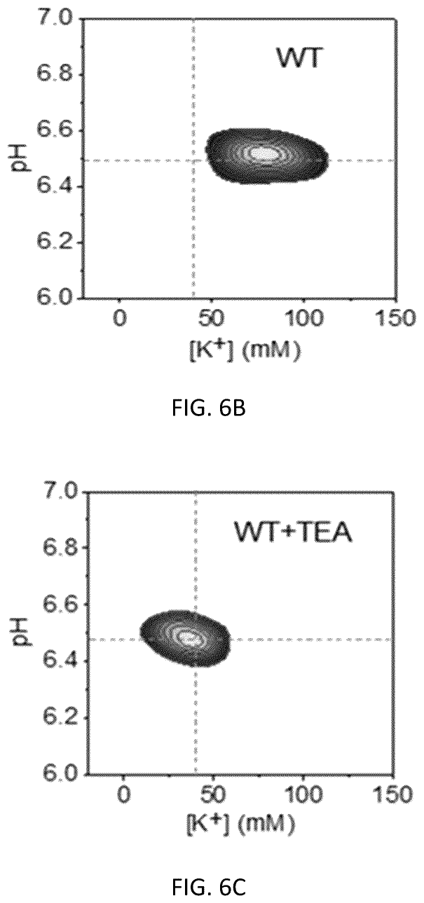

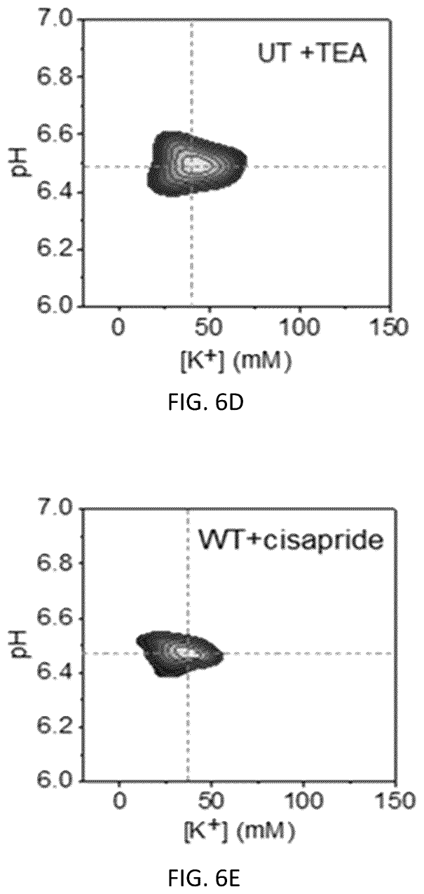

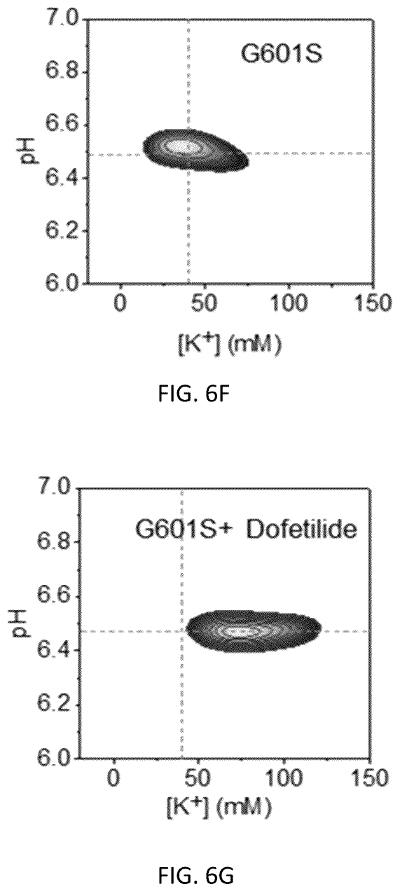

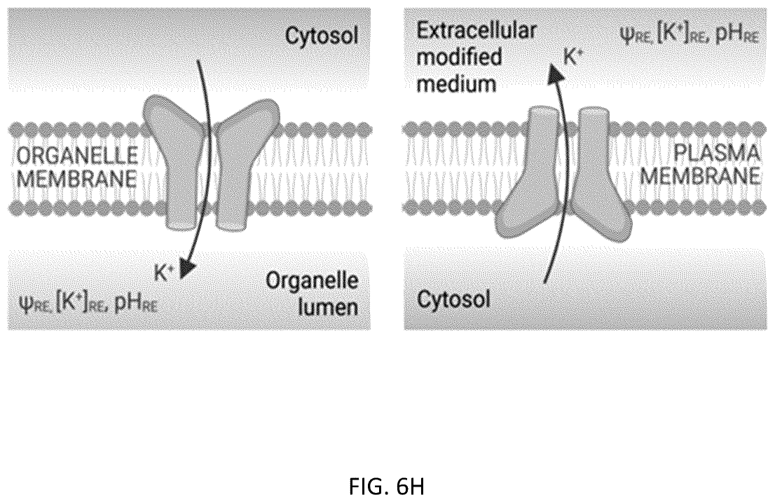

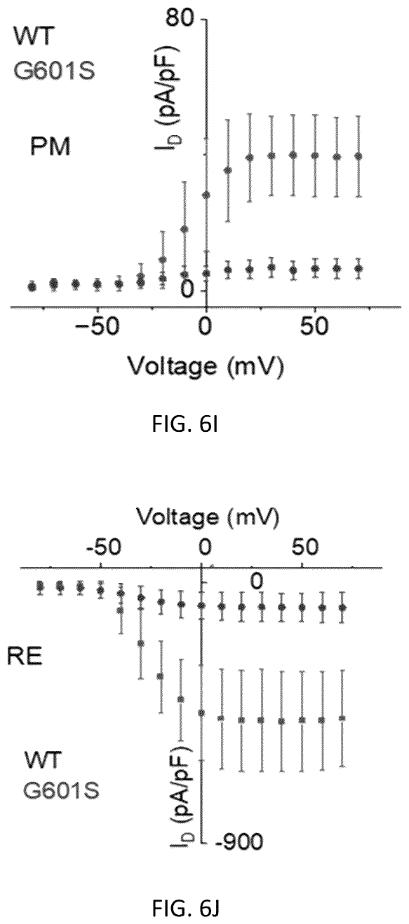

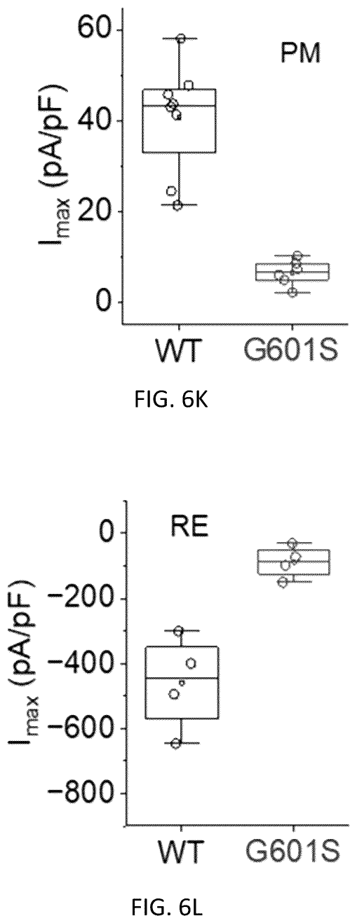

FIGS. 6A-6L illustrate pHlicKer probes channel activity, trafficking defects and rescue of trafficking. FIG. 6A shows a schematic of HEK 293T cells stably expressing Kv11.1 channels with a missense mutation (G601S) that cannot exit the endoplasmic reticulum (ER) unless cells are treated with dofetilide (Dof). FIGS. 6B-6G show [K⁺]_{RE} and pH values of recycling endosomes (REs) in WT cells without (FIG. 6B) and treated with tetraethyl ammonium chloride (TEA) (FIG. 6C), UT cells treated with TEA (FIG. 6D), WT cells treated with cisapride (FIG. 6E), G601S cells without (FIG. 6F), treated with dofetilide overnight and after wash out (FIG. 6G). FIG. 6H shows modifying the composition of the extracellular medium mimics, at the plasma membrane, the conditions experienced by Kv11.1 channels in REs. FIGS. 6I-6L show I-V curves (FIGS. 6I-6J) and maximal I_{Kv11.1} density (FIGS. 6K-6L) recorded from WT cells (black) and G601S cells (magenta) in standard extracellular saline (FIGS. 6I and 6K) or modified extracellular saline mimicking ion gradients across RE membranes (FIGS. 6J and FIG. 6L). I_{Kv11.1} were recorded by pre-pulsing cells from −80 to 70 mV followed by a test-pulse to −50 mV (i) or −100 mV (j). Peak I_{Kv11.1} measured during the test pulse as a function of the pre-pulse voltage to generate the I-V relations. Data are from one representative trial of independent experiments performed in triplicate (FIGS. 6B-6G), error bars represent standard deviations in FIGS. 6I-6L.

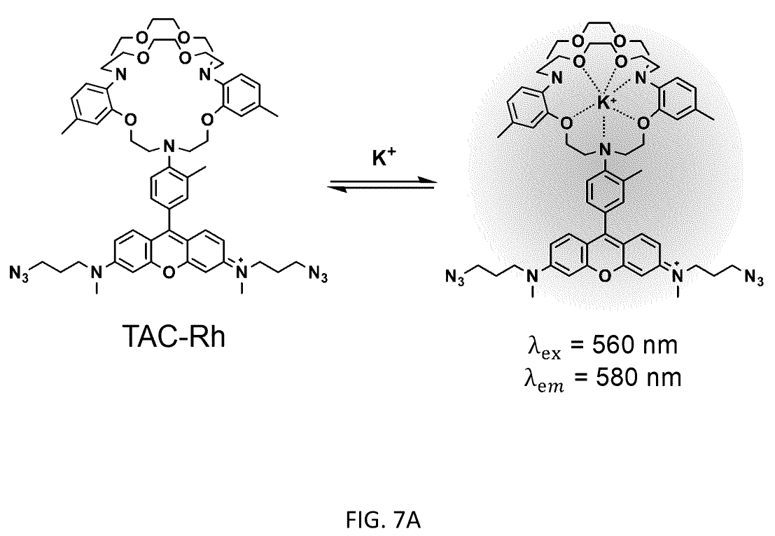

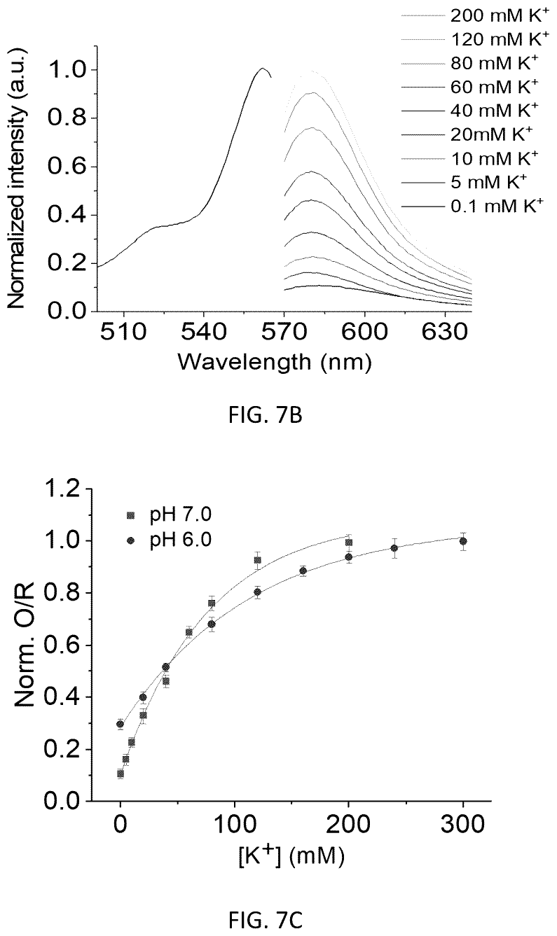

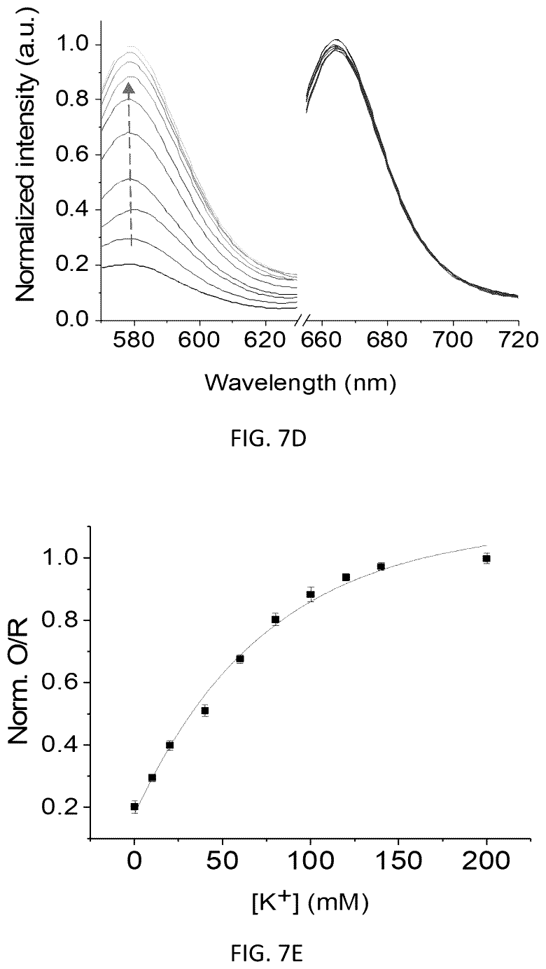

FIGS. 7A-7E illustrate TAC-Rh fluorescence response is a function of both pH and K⁺. FIG. 7A shows working principle of K⁺ sensing by TAC-Rh. FIG. 7B shows excitation (black) and emission (green) spectra of TAC-Rh. Emission intensity increased with increasing K⁺ concentration at pH=7.0. FIG. 7C shows normalized O/R ratio of TAC-Rh/Alexa Fluor 647 with increasing [K⁺] at pH 7.0 and 6.0. Error bar represents mean+s.e.m. of three independent experiments. FIG. 7D shows fluorescence emission spectra of pHlicKer^{RE} corresponding to TAC-Rh (green) and Alexa Fluor 647 (red) with increasing [K⁺] at pH=7.0. FIG. 7E shows normalized O/R ratio of TAC-Rh/Alexa Fluor 647 with increasing [K⁺] at pH 7.0 Error bar represents mean+s.e.m. of three independent experiments.

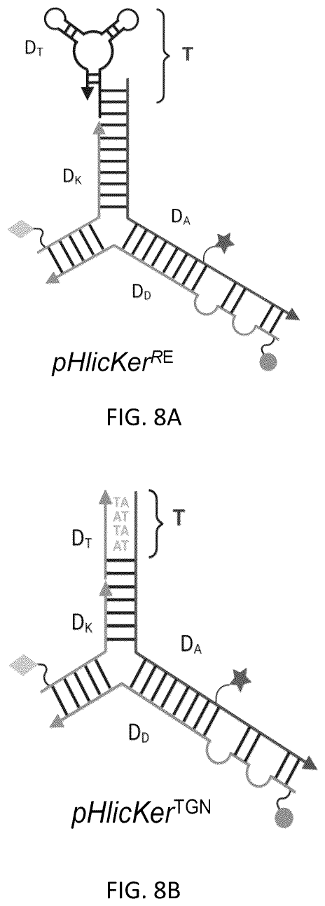

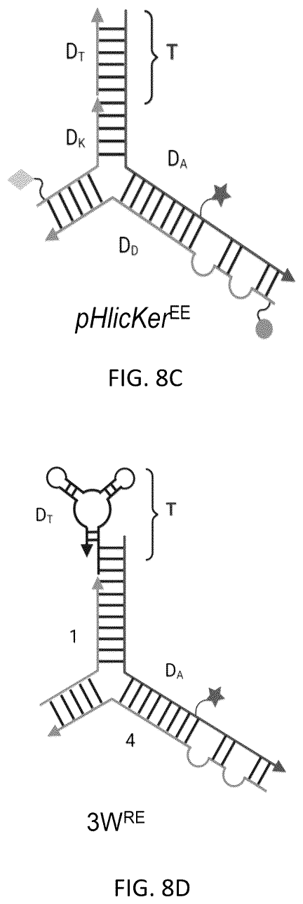

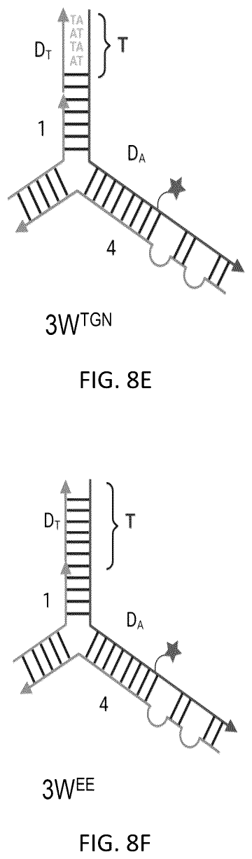

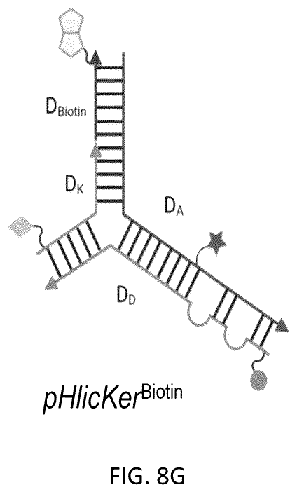

FIGS. 8A-8G illustrate Targeting modules (T) in organelle-specific pHlicKer variants. FIG. 8A shows pHlicKer^{RE} localizes in recycling endosomes (REs) by transferrin receptor (TR) mediated endocytosis. T is an aptamer that binds TfR. FIG. 8B shows pHlicKer^{TGN} is retrogradely trafficked by an scFv-furin chimera to the trans Golgi network (TGN). T is a d(AT)₄ sequence (cyan) in pHlicKer that sequence-specifically binds a single chain variable fragment (scFv) fused to the extracellular domain of furin. FIG. 8C shows pHlicKer^{EE} localizes in early endosomes (EEs) by scavenger receptor mediated endocytosis. T is a duplex DNA domain, which is an excellent ligand for scavenger receptors. FIGS. 8D-8F show three way (3W) junctions 3W^{RE}, 3W^{TGN} and 3W^{EE} are made from the same sequences as pHlicKer^{RE}, pHlicKer^{TGN} and pHlicKer^{EE} and lack the Alexa488 and TAC-Rh fluorophores for colocalization studies with fluorescent markers of the RE, TGN and EE. FIG. 8G shows pHlicKer^{Biotin} incorporates a biotin (grey pentagons) as indicated for immobilization on streptavidin coated beads.

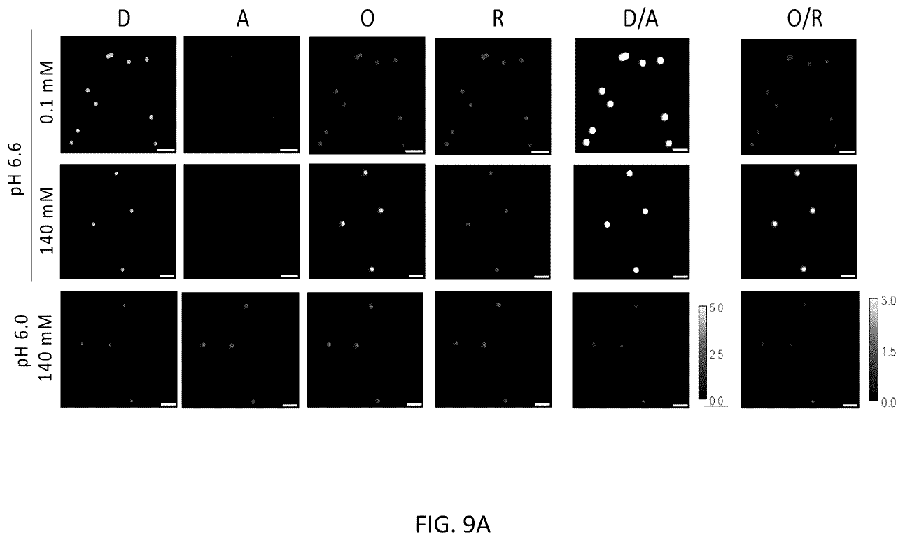

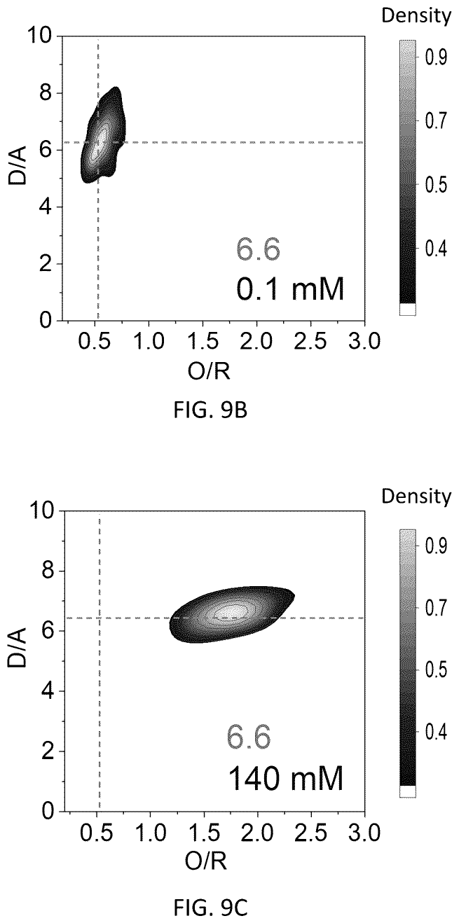

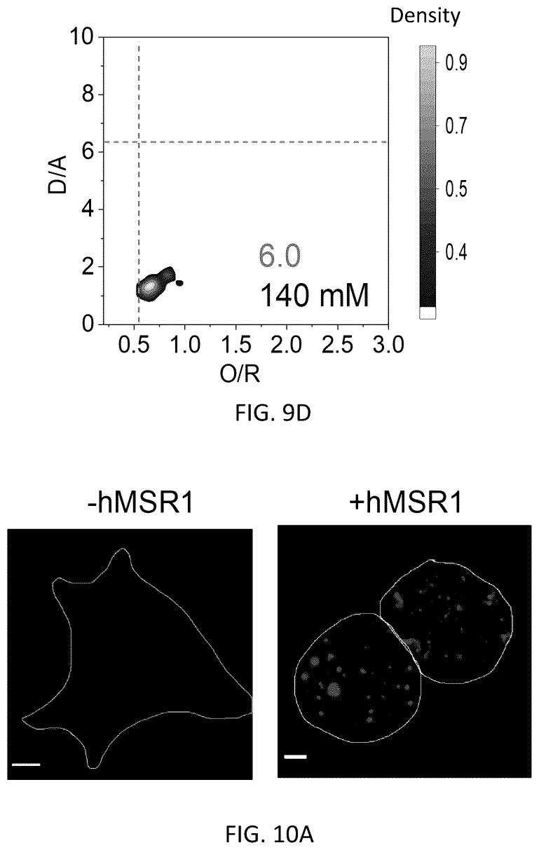

FIGS. 9A-9D illustrate calibration of pHlicKer on beads. FIG. 9A shows representative images of pHlicKer^{Biotin} on beads clamped at indicated pH and K⁺ levels, imaged in the donor channel (D), acceptor channel (A), TMR (O), and Alexa Fluor 647 (R) channels. D/A and O/R are the corresponding pixel-wise pseudocolor images. (n=100 beads). Scale bars, 5 μm. FIGS. 9B-9D show 2-IM profiles of beads clamped at indicated pH and [K⁺]. Experiments were performed in triplicate (n=10 cells, n=100 endosomes).

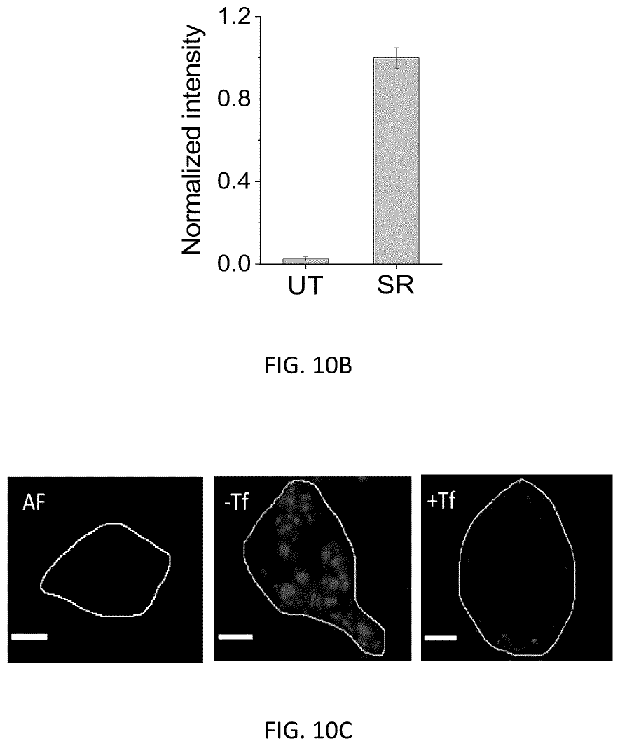

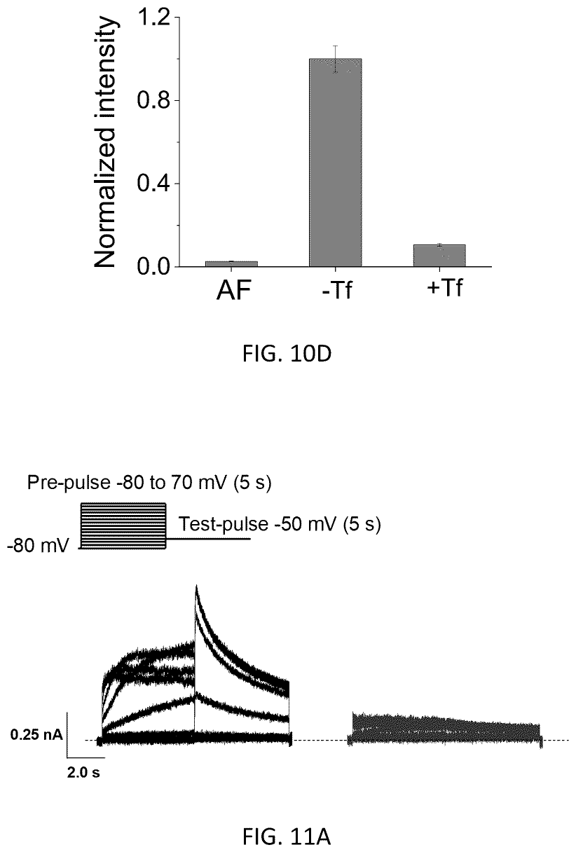

FIGS. 10A-10D illustrate Targetability of 3W^{EE} and 3W^{RE}. FIG. 10A shows uptake by HEK 293T cells expressing human scavenger receptor (hMSR1). Representative images of the uptake of Alexa 647 labelled 3W^{EE} in untransfected and hMSR1 transfected HEK 293T cells. Scale bars, 5 μm. FIG. 10B shows normalized whole cell intensities for (FIG. 10A). Data represent mean±s.e.m (n=15 cells). hMSR1 expressing HEK 293T cells showed effective internalization of pHlicKer^{EE}, revealing uptake is by scavenger receptors. FIG. 10C shows competition experiments with 3W^{RE} and excess unlabelled transferrin (Tf) in HEK 293T cells. Representative fluorescence images of HEK 293T cells pulsed with 3W^{RE} (500 nM) in the presence (+Tf, 20 μM) and absence (−Tf) of Tf. Cells are imaged in the Alexa 647 channel. AF, autofluorescence. Scale bars, 5 μm. FIG. 10D shows normalized intensities for (FIG. 10C). Data represent mean±s.e.m (n=15 cells). pHlicKer^{RE} internalization by HEK 293T cell is competed out by excess Tf, revealing that uptake is mediated by transferrin receptor mediated endocytosis.

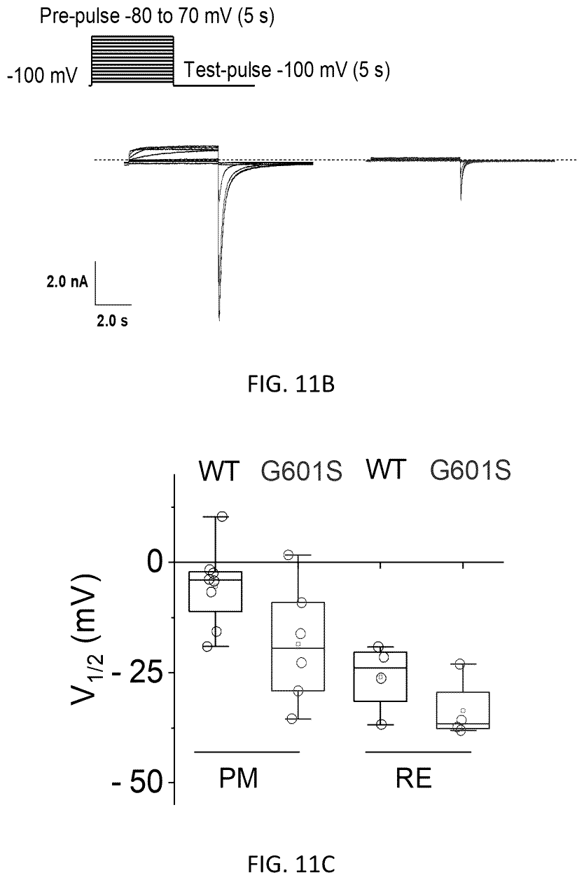

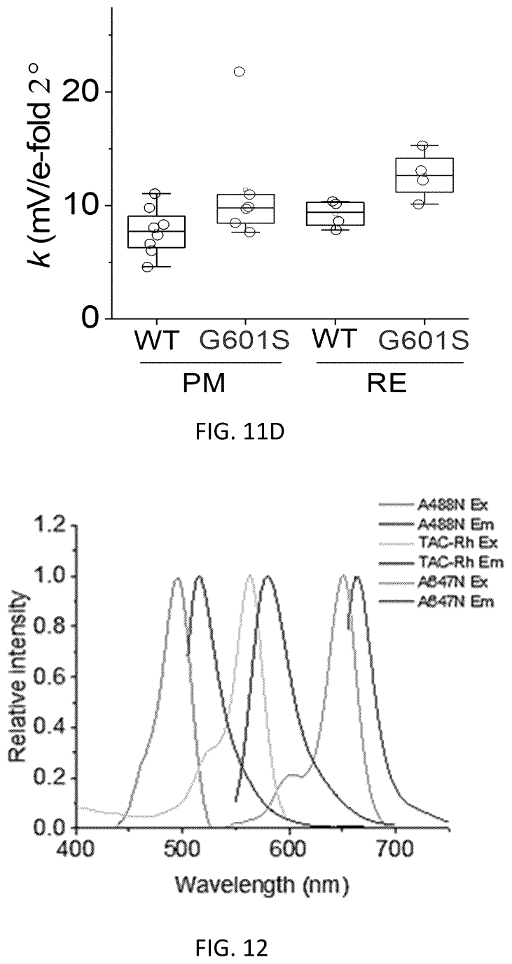

FIGS. 11A-11D illustrate Kv11.1 channel activity in transmembrane ion gradients equivalent to plasma membrane and recycling endosome. FIGS. 11A-11B show representative families of currents measured from cells stably expressing WT-Kv11.1 (black) or G601S-Kv11.1 (magenta) channel proteins using the voltage protocol shown in inset. FIG. 11A shows traces recorded using the standard extracellular saline, and FIG. 11B shows the modified extracellular saline to mimic recycling endosomes. Individual I-V relations were generated for each cell in each condition by plotting the peak current recorded during the test-pulse as a function of the pre-pulse. The individual I-V relations were described using a Boltzmann equation to calculate the IMAX, FIG. 11C shows midpoint potential for I_{Kv11.1} activation (V_{1/2}), or FIG. 11D shows the slope factor for I_{Kv11.1} current activation (k).

FIG. 12 illustrates spectral characteristics of pHlicKer probe. Normalized excitation and emission spectra of Alexa488N (blue), TAC-Rh (green) and Alexa647N (red). Fluorophores were chosen for their negligible spectral overlap.

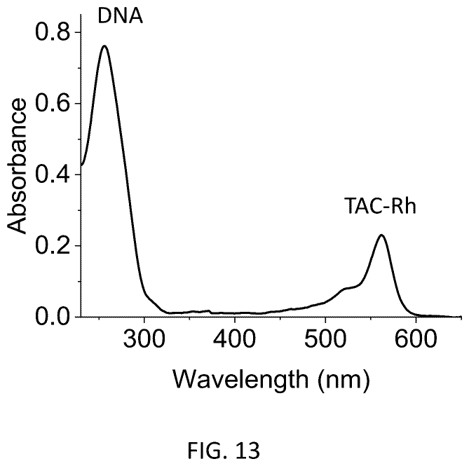

FIG. 13 illustrates the UV-Vis absorption spectrum of ssDNA-TAC-Rh (2.97 μM, in 100 mM phosphate buffer, pH 7.2 at 25° C.) showing a 1:1 labeling of K+-sensing fluorophore (TAC-Rh) to the 25-mer ssDNA-strand.

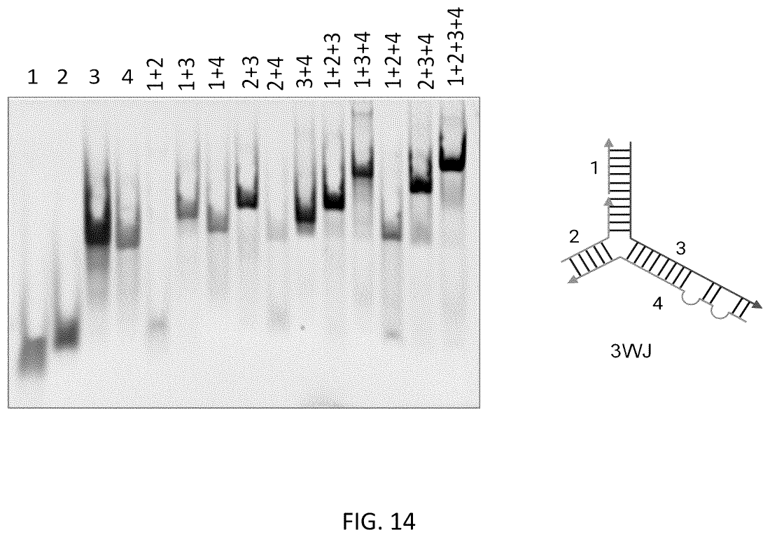

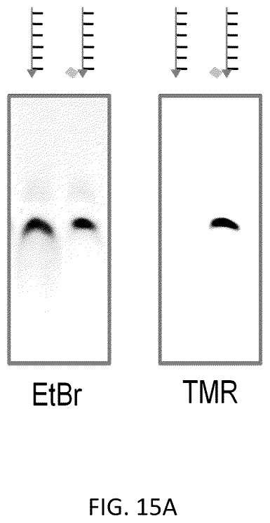

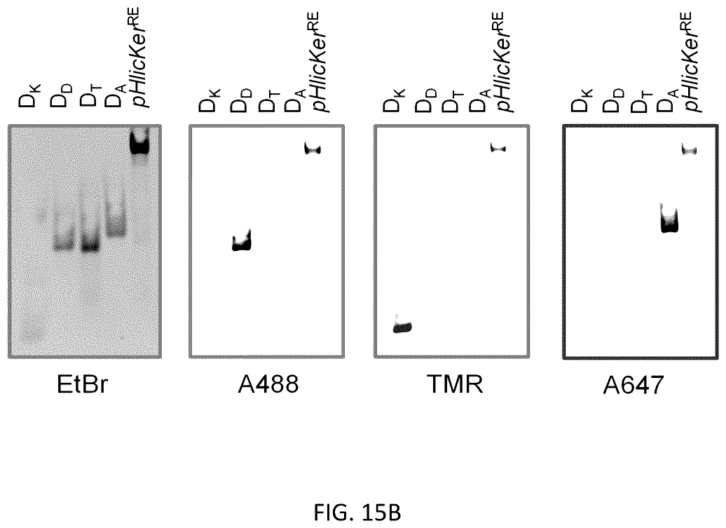

FIG. 14 illustrates the construction of three-way junction (3WJ). Native PAGE (10%) showing stepwise assembly of monomers, dimers, trimers and complete construction of 3WJ. Gels were visualized using EtBr staining. Experiments were performed in triplicate.

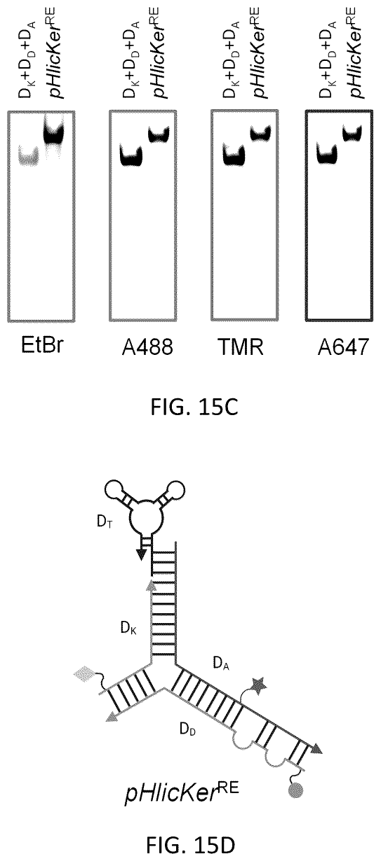

FIGS. 15A-15D illustrate characterization of pHlicKer^{RE}. FIG. 15A shows 15% Denaturing polyacrylamide gel electrophoresis in 1×TBE, showing the conjugation of TAC-Rh to D_{K}-DBCO strand. Gels were visualized in EtBr and TMR channels. FIGS. 15B-15C show 15% Native polyacrylamide gel electrophoresis showing formation of pHlicKer^{RE}. Gels were visualized in EtBr, Alexa Fluor 488, TMR and Alexa Fluor 647 channels. pHlicKer^{RE} displayed a band at higher molecular weight region distinct from the assembly formed from D_{K}, D_{D} and D_{A} (FIG. 15C). Experiments were performed in triplicate. FIG. 15D shows a schematic showing the components of pHlicKer^{RE}.

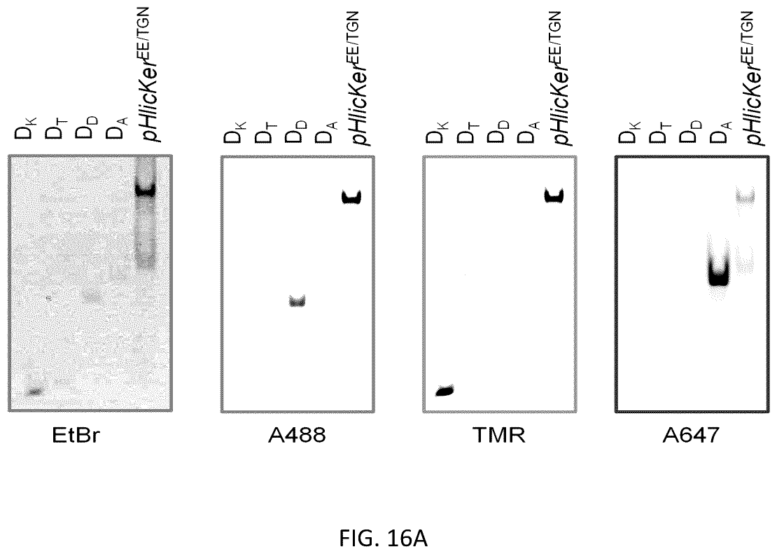

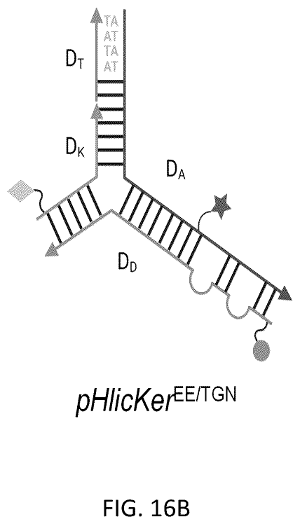

FIGS. 16A-16B illustrate characterization of pHlicKer^{EE/TGN}. FIG. 16A shows 15% Native PAGE showing formation of pHlicKer^{EE/TGN}. Gels were visualized in EtBr, Alexa Fluor 488, TMR and Alexa Fluor 647 channels. Experiments were performed in triplicate. FIG. 16B shows a schematic showing the components of pHlicKer^{EE/TGN}.

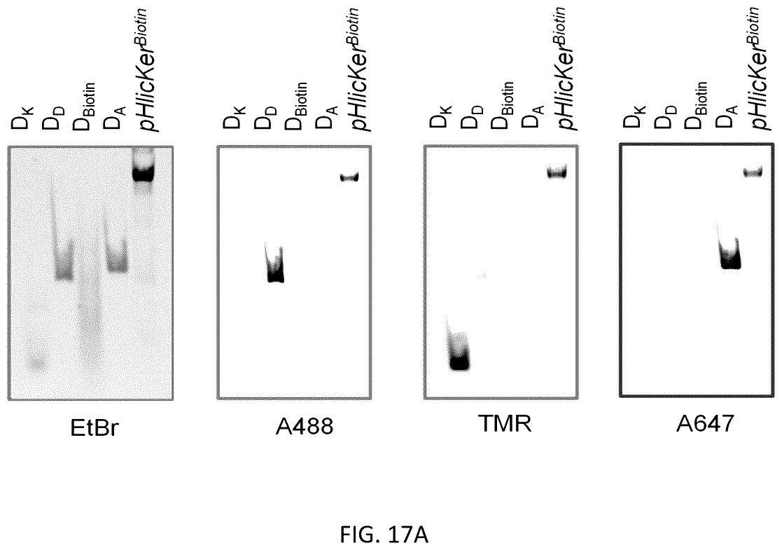

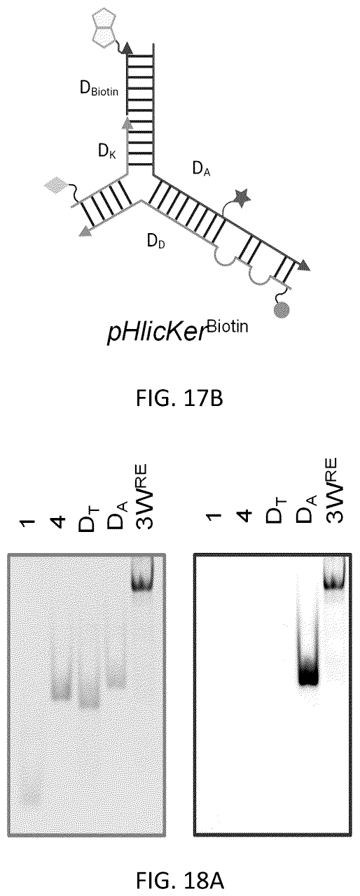

FIGS. 17A-17B illustrate characterization of pHlicKer^{Biotin}. FIG. 17A shows 15% Native PAGE showing formation of pHlicKer^{Biotin}. Gels were visualized in EtBr, Alexa Fluor 488, TMR and Alexa Fluor 647 channels. Experiments were performed in triplicate. FIG. 17B shows a schematic showing the components of pHlicKer^{biotin}.

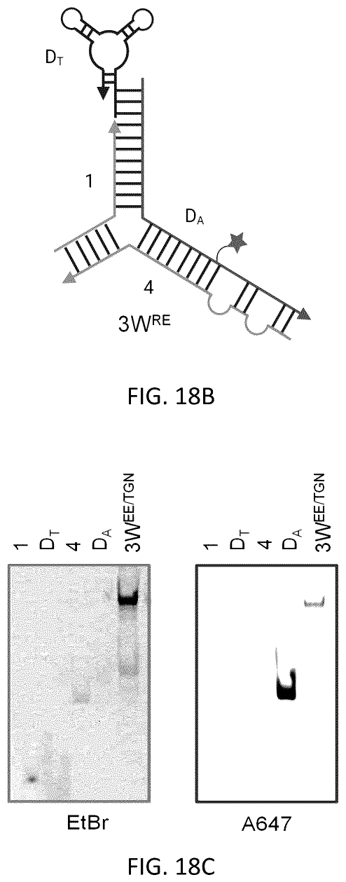

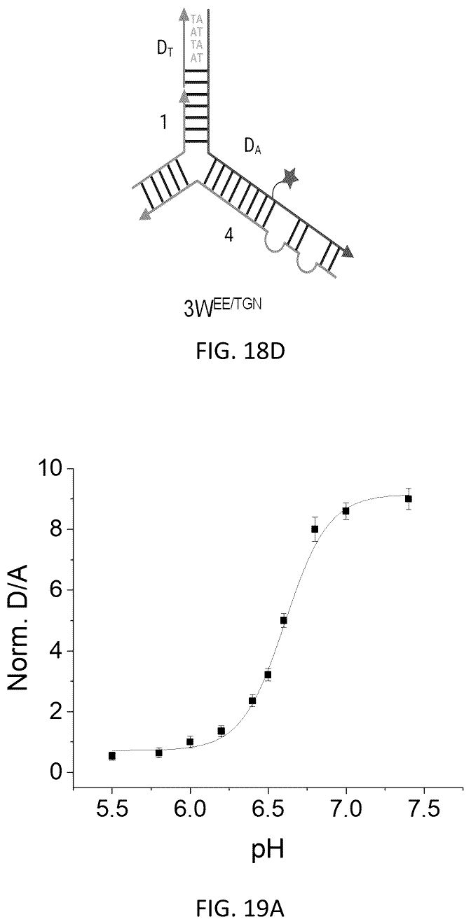

FIGS. 18A-18D illustrate characterization of 3W^{RE} and 3W^{EE/TGN}. In particular, FIGS. 18A and 18C show native PAGE showing formation of 3W^{RE} and 3W^{EE/TGN}. Gels were visualized in EtBr and Alexa Fluor 647 channels. Experiments were performed in triplicate. FIGS. 18B and 18D show schematics showing the components of 3W^{RE} and 3W^{EE/TGN}.

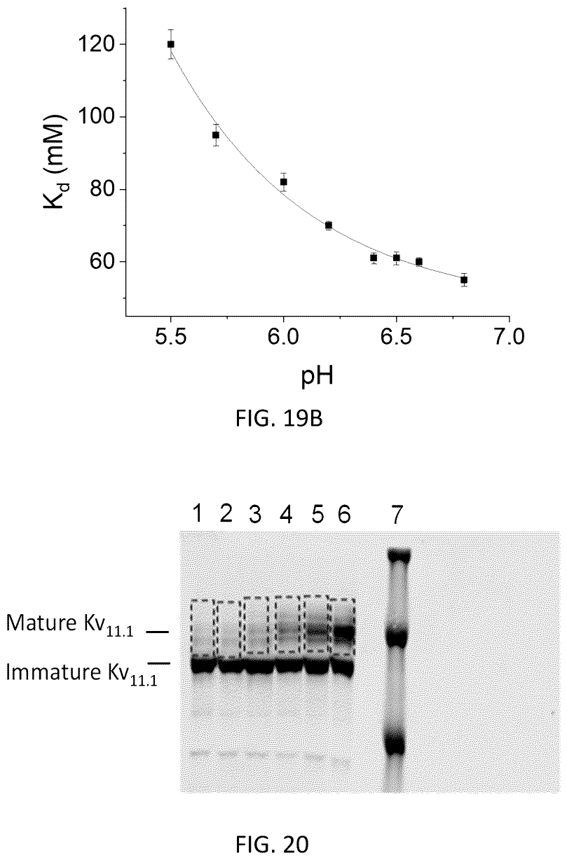

FIGS. 19A-19B illustrate normalized ratio of fluorescence intensity of donor to that of acceptor (D/A) of pHlicKer^{Biotin} on beads as a function of pH as shown in FIG. 19A, and pHlicKer^{Biotin} K_{d} as a function of pH. Error bars indicate mean±s. e. m. of n=3 experiments as shown in FIG. 19B.

FIG. 20 illustrates immunoblot analysis of lysates isolated from cells expressing G601S-Kv11.1 channels in control conditions (lane 1) or after overnight incubation in dofetilide (lane 2=1 nM, lane 3=10 nM, lane 4=100 nM, lane 5=1 μM, lane 6=10 μM). The immunoblots were probed with anti-Kv11.1. Lane 7 shows the molecular mass marker that corresponds to ≈250, ≈150, and ≈100 kDa. Incubating cells in higher dofetilide increases the density in the G601S-Kv11.1 channel protein band at ≈155 kDa. The 155 kDa Kv11.1 channel protein band corresponds to the complex N-linked glycosylated Kv11.1 channel protein (blue dashed squares)^{2,3 [NOW} 46, 47]. The data are similar to previous studies that show incubating cells expressing G601S-Kv11.1 in dofetilide increases the trafficking of G601S-Kv11.1 channels to the Golgi apparatus and cell surface membrane^{4[NOW 48]}.

### Nucleic Acid Complexes of the Disclosure

As provided above, one aspect of the disclosure includes nucleic acid complexes. The nucleic acid complexes of the disclosure as described herein include a K⁺ fluorophore conjugated to the first single-stranded nucleic acid molecule.

In certain embodiments, the nucleic acid complexes of the disclosure as describe herein include a K⁺ fluorophore conjugated to the first single-stranded nucleic acid molecule. Such K⁺ fluorophores may be ratiometric indicators. In embodiments, the K⁺ fluorophore is a triazacryptand K(+)-selective ionophore, RPS1. In one embodiment, the K⁺ fluorophore is a triazacryptand K(+)-selective ionophore. The triazacryptand K(+)-selective ionophore can be coupled to other molecules such as rhodamine, BODIPY, and naphthalimide. In certain embodiments of the disclosure, the K⁺ fluorophore is triazacryptand K(+)-selective ionophore is coupled to rhodamine (TAC-Rh). The TAC-Rh can include reactive groups such as azide (TAC-Rh-N₃) (see Scheme S3) that allows for conjugation or crosslinking of the K⁺ fluorophore to the first single-stranded nucleic acid molecule.

The K⁺ fluorophore as described herein can be crosslinked to the first single-stranded nucleic acid molecule using other linkers and methods known in the art. For example, the K⁺ fluorophore can be crosslinked using peptide chemistry, click chemistry, by forming ester, ether, thioether, disulfide, amine reactive N-Hydroxysuccinimidyl (NHS) esters, isocyanates, and isothiocyanates bonds, etc. In general, the K⁺ fluorophore is crosslinked to the first strand through a linker moiety stable under physiological conditions.

In certain embodiments, the present inventors have determined that the K⁺ fluorophore can be crosslinked to the first single-stranded nucleic acid molecule using click chemistry. Thus, in certain embodiments, the K⁺ fluorophore is crosslinked to the first strand through a triazole, thioether, or alkenyl sulfide group. For example, the triazole, thioether, or alkenyl sulfide group can be formed from an azide or thiol moiety on the K⁺ fluorophore and a alkyne or alkene moiety on the first strand. In another example, the triazole, thioether, or alkenyl sulfide group can be formed from an azide or thiol moiety on the first strand and a alkyne or alkene moiety on the K⁺ fluorophore.

In certain embodiments, the K⁺ fluorophore of the disclosure as described herein includes the following formula:

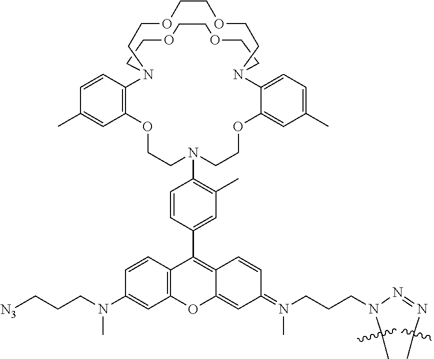

In certain embodiments of the disclosure, the first single-stranded nucleic acid molecule comprising a K⁺ fluorophore is of formula:

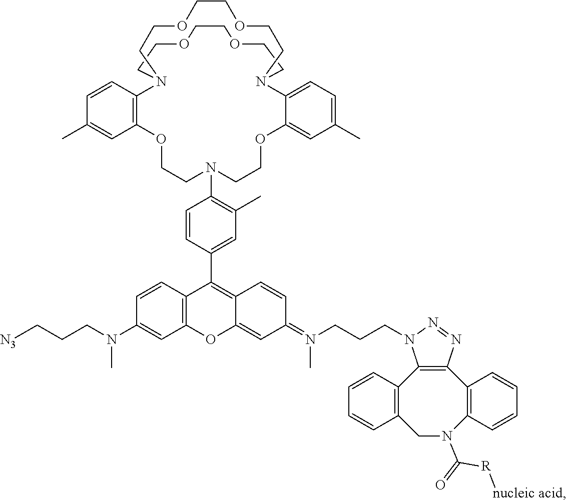

wherein R is a linker.

The oligonucleotides and nucleic acid molecules in the compositions and methods described herein may include one or more labels. Nucleic acid molecules can be labeled by incorporating moieties detectable by one or more means including, but not limited to, spectroscopic, photochemical, biochemical, immunochemical, or chemical assays. The method of linking or conjugating the label to the nucleotide or oligonucleotide depends on the type of label(s) used and the position of the label on the nucleotide or oligonucleotide.

As used herein, “labels” are chemical or biochemical moieties useful for labeling a nucleic acid. “Labels” include, for example, fluorescent agents, chemiluminescent agents, chromogenic agents, quenching agents, radionucleotides, enzymes, substrates, cofactors, inhibitors, nanoparticles, magnetic particles, and other moieties known in the art. Labels are capable of generating a measurable signal and may be covalently or noncovalently joined to an oligonucleotide or nucleotide.

In some embodiments, the nucleic acid molecules may be labeled with a “fluorescent dye” or a “fluorophore.” Dyes that may be used in the disclosed methods include, but are not limited to, the following dyes sold under the following trade names: 1,5 IAEDANS; 1,8-ANS; 4-methylumbelliferone; 5-carboxy-2,7-dichlorofluorescein; 5-carboxyfluorescein (5-FAM); 5-carboxytetramethylrhodamine (5-TAMRA); 5-hydroxy tryptamine (HAT); 5-ROX (carboxy-X-rhodamine); 6-carboxyrhodamine 6G; 6-JOE; 7-amino-4-methylcoumarin; 7-aminoactinomycin D (7-AAD); 7-hydroxy-4-methylcoumarin; 9-amino-6-chloro-2-methoxyacridine; ABQ; Acid Fuchsin; ACMA (9-amino-6-chloro-2-methoxyacridine); Acridine Orange; Acridine Red; Acridine Yellow; Acriflavin; Acriflavin Feulgen SITSA; Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC; AMCA-S; AMCA (Aminomethylcoumarin); AMCA-X; Aminoactinomycin D; Aminocoumarin; Aminomethylcoumarin (AMCA); Anilin Blue; Anthrocyl stearate; APC (Allophycocyanin); APC-Cy7; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9(Bisaminophenyloxadiazole); Berberine Sulphate; Beta Lactamase; BFP blue shifted GFP (Y66H); Blue Fluorescent Protein; BFP/GFP FRET; Bimane; Bisbenzamide; Bisbenzimide (Hoechst); Blancophor FFG; Blancophor SV; BOBO™-1; BOBO™-3;Bodipy 492/515; Bodipy 493/503; Bodipy 500/510; Bodipy 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy FL; Bodipy FL ATP; Bodipy FI-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; Calcein; Calcein Blue; Calcium Crimson™; Calcium Green; Calcium Orange; Calcofluor White; Cascade Blue™; Cascade Yellow; Catecholamine; CCF2 (GeneBlazer); CFDA; CFP—Cyan Fluorescent Protein; CFP/YFP FRET; Chlorophyll; Chromomycin A; CL-NERF (Ratio Dye, pH); CMFDA; Coelenterazine f; Coelenterazine fcp; Coelenterazine h; Coelenterazine hcp; Coelenterazine ip; Coelenterazine n; Coelenterazine O; Coumarin Phalloidin; C-phycocyanine; CPM Methylcoumarin; CTC; CTC Formazan; Cy2™; Cy3.18; Cy3.5™; Cy3™; Cy5.18; Cy5.5™; Cy5™; Cy7™; Cyan GFP; cyclic AMP Fluorosensor (FiCRhR); Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3; DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di-16-ASP); Dichlorodihydrofluorescein Diacetate (DCFH); DiD—Lipophilic Tracer; DiD (DiIC18 (5)); DIDS; Dihydorhodamine 123 (DHR); DiI (DiIC18 (3)); Dinitrophenol; weDiO (DiOC18 (3)); DiR; DIR (DiIC18 (7)); DNP; Dopamine; DsRed; DTAF; DY-630-NHS; DY-635-NHS; EBFP; ECFP; EGFP; ELF 97; Eosin; Erythrosin; Erythrosin ITC; Ethidium Bromide; Ethidium homodimer-1 (EthD-1); Euchrysin; EukoLight; Europium (III) chloride; EYFP; Fast Blue; FDA; Feulgen (Pararosaniline); Flazo Orange; Fluo-3; Fluo-4; Fluorescein (FITC); Fluorescein Diacetate; Fluoro-Emerald; Fluoro-Gold (Hydroxystilbamidine); Fluor-Ruby; FluorX; FM 1-43™; FM 4-46; Fura Red™; Fura Red™/Fluo-3; Fura-2; Fura-2/BCECF; Genacryl Brilliant Red B; Genacryl Brilliant Yellow 10GF; Genacryl Pink 3G; Genacryl Yellow 5GF; GeneBlazer (CCF2); GFP (S65T); GFP red shifted (rsGFP); GFP wild type, non-UV excitation (wtGFP); GFP wild type, UV excitation (wtGFP); GFPuv; Gloxalic Acid; Granular Blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1; Indodicarbocyanine (DiD); Indotricarbocyanine (DIR); Intrawhite Cf; JC-1; JO-JO-1; JO-PRO-1; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-Indo-1; Magnesium Green; Magnesium Orange; Malachite Green; Marina Blue; Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; NED™; Nitrobenzoxadidole; Noradrenaline; Nuclear Fast Red; Nuclear Yellow; Nylosan Brilliant lavin E8G; Oregon Green; Oregon Green 488-X; Oregon Green™; Oregon Green™ 488; Oregon Green™ 500; Oregon Green™ 514; Pacific Blue; Pararosaniline (Feulgen); PBFI; PE-Cy5; PE-Cy7; PerCP; PerCP-Cy5.5; PE-TexasRed [Red 613]; Phloxin B (Magdala Red); Phorwite AR; Phorwite BKL; Phorwite Rev; Phorwite RPA; Phosphine 3R; Phycoerythrin B [PE]; Phycoerythrin R [PE]; PKH26 (Sigma); PKH67; PMIA; Pontochrome Blue Black; POPO-1; POPO-3; PO-PRO-1; PO-PRO-3; Primuline; Procion Yellow; Propidium lodid (PI); PYMPO; Pyrene; Pyronine; Pyronine B; Pyrozal Brilliant Flavin 7GF; QSY 7; Quinacrine Mustard; Red 613 [PE-TexasRed]; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine Phalloidine; Rhodamine Red; Rhodamine WT; Rose Bengal; R-phycocyanine; R-phycoerythrin (PE); RsGFP; S65A; S65C; S65L; S65T; Sapphire GFP; SBFI; Serotonin; Sevron Brilliant Red 2B; Sevron Brilliant Red 4G; Sevron Brilliant Red B; Sevron Orange; Sevron Yellow L; sgBFP™; sgBFP™ (super glow BFP); sgGFP™; sgGFP™ (super glow GFP); SITS; SITS (Primuline); SITS (Stilbene Isothiosulphonic Acid); SNAFL calcein; SNAFL-1; SNAFL-2; SNARF calcein; SNARF1; Sodium Green; SpectrumAqua; SpectrumGreen; SpectrumOrange; Spectrum Red; SPQ (6-methoxy-N-(3-sulfopropyl)quinolinium); Stilbene; Sulphorhodamine B can C; Sulphorhodamine G Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; TET™; Tetracycline; Tetramethylrhodamine (TRITC); Texas Red™; Texas Red-X™ conjugate; Thiadicarbocyanine (DiSC3); Thiazine Red R; Thiazole Orange; Thioflavin 5; Thioflavin S; Thioflavin TCN; Thiolyte; Thiozole Orange; Tinopol CBS (Calcofluor White); TMR; TO-PRO-1; TO-PRO-3; TO-PRO-5; TOTO-1; TOTO-3; TriColor (PE-Cy5); TRITC TetramethylRodaminelsoThioCyanate; True Blue; TruRed; Ultralite; Uranine B; Uvitex SFC; VIC®; wt GFP; WW 781; X-Rhodamine; XRITC; Xylene Orange; Y66F; Y66H; Y66W; Yellow GFP; YFP; YO-PRO-1; YO-PRO-3; YOYO-1; YOYO-3; pHrodo™ (available from Thermo Fischer Scientific, Inc. Waltham, MA), and salts thereof.

Fluorescent dyes or fluorophores may include derivatives that have been modified to facilitate conjugation to another reactive molecule. As such, fluorescent dyes or fluorophores may include amine-reactive derivatives such as isothiocyanate derivatives and/or succinimidyl ester derivatives of the fluorophore.

The nucleic acid molecules of the disclosed compositions and methods may be labeled with a quencher. Quenching may include dynamic quenching (e.g., by FRET), static quenching, or both. Illustrative quenchers may include Dabcyl. Illustrative quenchers may also include dark quenchers, which may include black hole quenchers sold under the tradename “BHQ” (e.g., BHQ-0, BHQ-1, BHQ-2, and BHQ-3, Biosearch Technologies, Novato, Calif.). Dark quenchers also may include quenchers sold under the tradename “QXL™” (Anaspec, San Jose, Calif.). Dark quenchers also may include DNP-type non-fluorophores that include a 2,4-dinitrophenyl group.

In some embodiments, it may be useful to include interactive labels on two or more oligonucleotides with due consideration given for maintaining an appropriate spacing of the labels on the nucleic acid molecules to permit the separation of the labels during a conformational change in the nucleic acid complex. One type of interactive label pair is a quencher-dye pair, which may include a fluorophore and a quencher. The ordinarily skilled artisan can select a suitable quencher moiety that will quench the emission of the particular fluorophore. In an illustrative embodiment, the Dabcyl quencher absorbs the emission of fluorescence from the fluorophore moiety.

In some embodiments, the proximity of the two labels can be detected using FRET or fluorescence polarization. FRET is a distance-dependent interaction between the electronic excited states of two dye molecules in which excitation is transferred from a donor molecule to an acceptor molecule without emission of a photon. Examples of donor/acceptor dye pairs for FRET are known in the art and may include fluorophores and quenchers described herein such as Fluorescein/Tetramethyl-rhodamine, IAEDANS/Fluorescein (Molecular Probes, Eugene, Oreg.), EDANS/Dabcyl, Fluorescein/Fluorescein (Molecular Probes, Eugene, Oreg.), BODIPY FL/BODIPY FL (Molecular Probes, Eugene, Oreg.), BODIPY TMR/ALEXA 647, ALEXA-488/ALEXA-647, and Fluorescein/QSY7™.

The labels can be conjugated to the nucleic acid molecules directly or indirectly by a variety of techniques. Depending upon the precise type of label used, the label can be located at the 5′ or 3′ end of the oligonucleotide, located internally in the oligonucleotide's nucleotide sequence, or attached to spacer arms extending from the oligonucleotide and having various sizes and compositions to facilitate signal interactions. Using commercially available phosphoramidite reagents, one can produce nucleic acid molecules containing functional groups (e.g., thiols or primary amines) at either terminus, for example by the coupling of a phosphoramidite dye to the 5′ hydroxyl of the 5′ base by the formation of a phosphate bond, or internally, via an appropriately protected phosphoramidite.

Nucleic acid molecules may also incorporate functionalizing reagents having one or more sulfhydryl, amino or hydroxyl moieties into the nucleic acid sequence. For example, a 5′ phosphate group can be incorporated as a radioisotope by using polynucleotide kinase and [γ32P] ATP to provide a reporter group. Biotin can be added to the 5′ end by reacting an aminothymidine residue, introduced during synthesis, with an N-hydroxysuccinimide ester of biotin. Labels at the 3′ terminus, for example, can employ polynucleotide terminal transferase to add the desired moiety, such as for example, cordycepin, 35S-dATP, and biotinylated dUTP.

Oligonucleotide derivatives are also available as labels. For example, etheno-dA and etheno-A are known fluorescent adenine nucleotides which can be incorporated into a reporter. Similarly, etheno-dC is another analog that can be used in reporter synthesis. The reporters containing such nucleotide derivatives can be hydrolyzed to release much more strongly fluorescent mononucleotides by the polymerase's 5′ to 3′ nuclease activity as nucleic acid polymerase extends a primer during PCR.

In some embodiments, a first label is conjugated to the second single-stranded nucleic acid molecule and a second label is conjugated to the third single-stranded nucleic acid molecule. In embodiments, the first label and second label are members of a FRET pair and wherein the intensity of the signal varies as a function of the conformation of the nucleic acid complex.

In certain embodiments, the intensity of the signal is irrelevant of the distance between the first and second labels and/or the relative orientation of the first and second labels.

In certain embodiments, the intensity of the signal varies as a function of at least one of the distance between the first and second labels and the relative orientation of the first and second labels.

In some embodiments, the first and second labels comprise a donor and acceptor pair. In some embodiments, the signal is measured using a FRET technique. For example, the signal can be measured at 2 different wavelengths. In another example, the signal can be measured at 4 different wavelengths. In some embodiments, at least one label is selected from the group consisting of an Atto dye, an Alexa Flour® dye, a Cy® dye, and a BODIPY dye. In some embodiments, the donor and acceptor pair are FITC and TRITC, Cy3 and Cy5, or Alexa-488 and Alexa-647. In some embodiments, the donor and acceptor pair are labels described herein. In some embodiments, the first and second label comprise a donor fluorophore and an acceptor quencher.

In some embodiments, the signal and label is directionally dependent (anisotropy). Non-limiting examples of such labels include Atto dyes, BODipy dyes, Alexa dyes, TMR/TAMRA dyes, or Cy dyes.

A provided above, the nucleic acid complexes of the disclosure include quaternary single-stranded nucleic acid molecules including a first single-stranded molecule, a second single-stranded nucleic acid molecule, a third single-stranded nucleic acid molecule and a fourth single-stranded nucleic acid molecule, wherein the first single-stranded nucleic acid molecule is partially complementary to the second single-stranded molecule and the third single-stranded nucleic acid molecule; the second single-stranded nucleic acid molecule is partially complementary to the first and third single-stranded nucleic acid molecules; the third single-stranded nucleic acid molecule is partially complementary to the first single-stranded nucleic acid molecule, the second single-stranded nucleic acid molecule and the fourth single-stranded nucleic acid molecule.

As defined herein, a nucleic acid strand is fully complementary when all bases are capable of forming conventional Watson-Crick base-pairing (e.g. G-C and A-T base pairing). A nucleic acid strand is partially complementary when at least one of the base pairs is not complementary to the opposing strand.

In certain embodiments of the nucleic acid complexes of the disclosure, each of the first single-stranded nucleic acid molecule the second single-stranded nucleic acid molecule, and/or the third single-stranded nucleic acid molecule is independently less than 200 nucleotides. In some embodiments, each of the first single-stranded nucleic acid molecule, the second single-stranded nucleic acid molecule, the third single-stranded nucleic acid molecule, and the fourth single-stranded nucleic acid molecule is independently less than, at least, or exactly 20, 30, 40, 60, 80, 100, 125, 150, 175, 200 nucleotides in length, or any derivable range therein.

The nucleic acid complexes described herein are useful as K⁺ concentration sensors, and have high sensitivity without a substantial change in cooperativity. In certain embodiments, the nucleic acid complexes described herein are capable of determining the K⁺ concentration in a range of 10 nM to 10 mM, the range is inclusive of the recited K⁺ concentration. For example, in certain embodiments, the nucleic acid complexes described herein are capable of determining the K⁺ concentration in a range of 0.1 to 1 mM, or 1 mM to 10 mM, 10 mM to 100 mM, or 100 mM to 300 mM. In other embodiments, the recited K⁺ concentration is excluded.

The nucleic acid complexes described herein are also useful as pH sensors, and have high sensitivity without a substantial change in cooperativity. In certain embodiments, the nucleic acid complexes described herein are capable of determining the pH of 7.0 or less. In certain embodiments, the nucleic acid complexes described herein are capable of determining the pH of less than or exactly pH 7.0, 6.9, 6.8, 6.7, 6.6, 6.5, 6.4, 6.3, 6.2, 6.1, 6.0, 5.9, 5.8 (or any derivable range therein).

Cytosine rich DNA sequences are found in human genomes such as in telomeres and in promoters of several oncogenes, e.g., c-myc. In certain embodiments, the nucleic acid complexes of the disclosure include single stranded nucleic acid molecules (such as the second single stranded nucleic acid molecule and third single stranded nucleic acid molecule) can form a special tetraplex structure under slightly acidic condition where two parallel duplexes paired through C.CH+ pairs intercalated with each other in head to tail orientation called the i-motif. The “i-motif” is a nucleic acid (DNA and/or RNA) containing complex characterized by the presence of cytosine-rich stretches or stretches rich in cytosine derivatives, including two parallel-stranded duplexes in which the cytosines or derivatives thereof form base pairs, and the two duplexes are associated anti-parallel to one another. The pairs of cytosine or derivatives thereof of one duplex are intercalated with those of the other duplex.

The structure of an i-motif differs from that of the usual DNA duplex because the base pairing scheme involves hemiprotonated cytosines which result in the formation of C.C+ base pairs. Specifically, one of the cytosines contained in each pair is protonated. The i-motif may also exist as a tetramer formed by the association of two duplexes as described above.

In certain embodiments, the nucleic acid complexes of the disclosure may be synthesized from oligonucleotide sequences including a stretch of at least two, at least three, or at least four consecutive cytosines. By modifying the number of cytosines, as well as the degree of complementarity between both strands, it is possible to modulate the response time of the nucleic acid complexes of the disclosure and to the pH sensing range. When more cytosines contribute to the i-motif, the stability of the motif is increased. Moreover, this motif may be formed by the interaction of stretches containing different numbers of cytosines. Furthermore, a cytosine-rich stretch may contain one or two non-cytosine base(s) in between the cytosines. However, this may reduce the stability of the i-motif. The cytosine stretches which comprise the i-motif may belong to different strands of nucleic acids; however, any two of them may also be linked together covalently or non-covalently. Also, any two of them may be part of a single nucleic acid strand wherein they are separated by a stretch of specified bases.

The nucleic acid complexes described herein are useful as pH sensors, and have high sensitivity (as evidenced by fold change of D/A ratio) without a substantial change in cooperativity. In some embodiments, the method further comprises calculating a D/A ratio from the signal intensity values. In some embodiments, the D/A ratio is a normalized value. In some embodiments, the fold change of the D/A ratio is at least 4.1, 5, 6, 7, or 7.5. In some embodiments, the fold change of the D/A ratio is between 4.1 and 7.5, between 5 and 7.5, between 6 and 7.5, between 7 and 7.5, between 4.1 and 7, between 5 and 7, between 6 and 7, between 4.1 and 6, between 5 and 6, or between 4.1 and 5. In some embodiments, the fold change of the D/A ratio is at least or exactly 4.5, 5, 6, 7, 7.5, 8, 8.5, 9, 9.5, 10, 10.5, 11, 11.5, 12, 12.5, 13, or 14 (or any derivable range therein). In some embodiments, the cooperativity, compared to the unmodified nucleic acid complexes, is changed less than 2 fold, or less than 1.75, 1.5, 1.25. 1, 0.75. 0.5, 0.25, 0.2, 0.1, fold or any derivable range therein. In some embodiments, the cooperativity is less than 5, 10, 15, 20, 25, 30, 35, 40, 45, or 50% different than the un-modified nucleic acid complexes. In some embodiments, the fold change of the D/A ratio is at least or exactly 4.5, 5, 6, 7, 7.5, 8, 8.5, 9, 9.5, 10, 10.5, 11, 11.5, 12, 12.5, 13, or 14 (or any derivable range therein) and the cooperativity, compared to the unmodified nucleic acid complexes, is changed by less than 1.75, 1.5, 1.25. 1, 0.75. 0.5, 0.25, 0.2, or 0.1 fold or any derivable range therein. In some embodiments, the pH_{half} is altered without substantially increasing the cooperativity. In some embodiments, the pH_{half} is at least, at most, or exactly 5.0, 5.2, 5.4, 5.6, 5.8, 6.0, 6.2, 6.4, 6.6, 6.8, 7.0, 7.2, 7.4, 7.6, 7.8, 8.0, 8.2, 8.4, 8.6, 8.8, or 9.0 (or any derivable range therein). In some embodiments, the pH_{half}, compared to the un-modified nucleic acid complexes, is at least, at most, or exactly 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 2.9, or 3.0 pH units different (or any range derivable therein). In some embodiments, the pH_{half}, compared to the un-modified nucleic acid complexes, is at least, at most, or exactly 3, 5, 6, 8, 10, 12, 14, 16, 18, 20, 22, 24, 24, 26, 28, or 30% different (or any derivable range therein). In some embodiments, the pH_{half}, compared to the un-modified nucleic acid complexes, is at least, at most, or exactly 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 2.9, or 3.0 pH units different or is at least, at most, or exactly 3, 5, 6, 8, 10, 12, 14, 16, 18, 20, 22, 24, 24, 26, 28, or 30% different (or any derivable range therein) and the cooperativity, compared to the unmodified nucleic acid complexes, is changed by less than 1.75, 1.5, 1.25. 1, 0.75. 0.5, 0.25, 0.2, or 0.1 fold or any derivable range therein. In some embodiments, the measured value described herein (i.e., signal intensity, pH_{half}, fold change, or cooperativity) is a normalized value.

In some embodiments, the nucleic acid complex includes second and third single-stranded nucleic acid molecules are capable of forming an i-motif under acidic conditions.

The nucleic acid molecules and complexes of the disclosure, in some embodiments, comprise a targeting moiety, such as a nucleic acid, small molecule, or polypeptide that has an affinity for a certain target or, by virtue of its chemical makeup, is targeted to a particular location in the cell. The targeting moiety can act as a handle to target the nucleic acid complexes of the disclosure to different subcellular locations. The targeting moiety may be a nucleic acid that binds to a receptor protein, and the receptor protein may be one that is intracellularly targeted or conjugated to a protein that is intracellularly targeted. The targeting moiety or receptor protein may be a targeting nucleic acid or a protein such as a plasma membrane protein that is endocytosable, any proteins that possess a natural receptor, a protein that traffics between intracellular locations via the plasma membrane, toxins, viruses and viral coat proteins, cell penetrating peptides, signal sequences, intracellular targeting sequences, small organic molecules, endocytic ligands and trafficking proteins. In some embodiments, the targeting moiety is an aptamer, a duplex domain targeted to an artificial protein receptor, a nucleic acid sequence that binds an anionic-ligand binding receptor, or an endocytic ligand. The targeting moiety may also be a G4 core sequence or ribozyme.

In some embodiments, the targeting moiety is a nucleic acid sequence. In some embodiments, the targeting moiety has a cognate artificial protein receptor. The artificial receptor may be, for example, a single chain variable fragment (scFv), transcription factor, Zn-fingered protein, leucine zipper, or DNA binding immunoglobulin, In some embodiments, the targeting moiety is encoded on the same nucleic acid strand as the first and/or second single-stranded nucleic acid molecule. In some embodiments, the targeting moiety is selected from an aptamer, a duplex domain targeted to an artificial protein receptor, a nucleic acid sequence that binds an anionic-ligand binding receptor, and an endocytic ligand. In some embodiments, the targeting moiety comprises a peptide directly or indirectly conjugated to the nucleic acid molecule. In some embodiments, the targeting moiety peptide comprises one or more of a fusogenic peptide, a membrane-permeabilizing peptide, a sub-cellular localization sequence, or a cell-receptor ligand. In some embodiments, the sub-cellular localization sequence targets the nucleic acid complex to a region of the cell where spatial localization of a targeted protein is present. In some embodiments, the sub-cellular localization sequence targets the nucleic acid complex to a region of the cell selected from the group consisting of: early endosome, late endosome, plasma membrane, recycling endosome, the endoplasmic reticulum, cis Golgi network, trans-Golgi network, lysosome, peroxisome, and secretory vesicles.

In some embodiments, the nucleic acid is a peptide nucleic acid (PNA).

The current methods, nucleic acids, and nucleic acid complexes may be used in combination with additional nucleic acid based sensors, such as those described in International Patent Publication No, WO 2015/159122, which is herein incorporated by reference.

In the present disclosure, PNA or PNA strand or PNA sequence is used interchangeably and has the same scope or meaning. In the present disclosure, DNA or DNA strand or DNA sequence is used interchangeably and has the same scope or meaning. In the present disclosure, RNA or RNA strand or RNA sequence is used interchangeably and has the same scope or meaning.

### Diseases Detection and Monitoring

The methods, compositions, nucleic acid complexes, and kits of the disclosure can be used for the detection of diseases, the monitoring of diseases, and as a drug screening platform. In some embodiments of the disclosure, the disease is characterized as a K+ ion channel disorder or disease. Abnormalities in K(+) channels are associated with diseases like long QT syndrome, Anderson Tawil syndrome, epilepsy, type 2 diabetes mellitus, etc. A number of naturally occurring as well as synthetic compounds have been identified that modulate the opening and closure of K(ATP) Channels. Some of the currently available K(+) channel modulators like sulphonylureas, minoxidil, amiodarone, etc. lack tissue selectivity and have adverse effects. Hence, the success of K(ATP) channel modulators depend on their tissue selectivity. The nucleic acid complexes of the present disclosure can be used to study K(+) channels as this can lead to the development of newer drugs with tissue selectivity for various diseases.

### Kits

The materials and components described for use in the methods may be suited for the preparation of a kit. Thus, the disclosure provides a detection kit useful for determining the pH and the presence, absence, or concentration of K⁺ in a sample, cell or region thereof. Specifically, the technology encompasses kits for measuring the pH and K⁺ of one or more cells in a sample. The kit can comprise a nucleic acid complexes as described herein.

In some embodiments, the methods described herein may be performed by utilizing pre-packaged diagnostic kits comprising the necessary reagents to perform any of the methods of the technology. For example, such a kit would include a detection reagent for measuring the pH and K⁺ of a cell or region thereof. In one embodiment of such a kit, the detection reagents are the nucleic acid complexes of the disclosure. Oligonucleotides are easily synthesized and are stable in various formulations for long periods of time, particularly when lyophilized or otherwise dried to a powder form. In this form, they are easily reconstituted for use by those of skill in the art. Other reagents and consumables required for using the kit could be easily identified and procured by those of skill in the art who wish to use the kit. The kits can also include buffers useful in the methods of the technology. The kits may contain instructions for the use of the reagents and interpreting the results.

In some embodiments, the technology provides a kit comprising at least one sample (e.g., a pH standard and/or a K⁺ concentration standard) packaged in one or more vials for use as a control. Each component of the kit can be enclosed within an individual container and all of the various containers can be within a single package, along with instructions for performing the assay and for interpreting the results of the assays performed using the kit.

In some embodiments, the kit comprises a device for the measurement of pH and K⁺ in a sample. In some embodiments, the kit comprises a device for the measurement of pH and Cl⁻ in a sample. In some embodiments, the device is for measuring pH and/or analyte in cell culture or in whole, transparent organisms (e.g., C. elegans).

### Definitions

Throughout this specification, unless the context requires otherwise, the word “comprise” and “include” and variations (e.g., “comprises,” “comprising,” “includes,” “including”) will be understood to imply the inclusion of a stated component, feature, element, or step or group of components, features, elements or steps but not the exclusion of any other integer or step or group of integers or steps.

As used in the specification and the appended claims, the singular forms “a,” “an” and “the” include plural referents unless the context clearly dictates otherwise.

As used herein, a “fluorescent dye” or a “fluorophore” is a chemical group that can be excited by light to emit fluorescence. Some fluorophores may be excited by light to emit phosphorescence. Dyes may include acceptor dyes that are capable of quenching a fluorescent signal from a fluorescent donor dye.

As used herein, “crosslinked” or “conjugated” refers to a covalent connection between the nucleic acid molecule and another moiety of interest, such as the K⁺ fluorophore. In certain embodiments, the crosslink between the nucleic acid molecule and this moiety is water compatible. In certain embodiments, the crosslink between the nucleic acid molecule and this moiety is stable under physiological conditions.

As used herein, “nucleic acid,” “nucleotide sequence,” or “nucleic acid sequence” refer to a nucleotide, oligonucleotide, polynucleotide, or any fragment thereof and to naturally occurring or synthetic molecules. The term “peptide nucleic acid” or “PNA” as used herein generally refers to nucleic acid analogue in which the sugar phosphate backbone of natural nucleic acid has been replaced by a synthetic peptide backbone. The term “RNA equivalent” in reference to a DNA sequence, is composed of the same linear sequence of nucleotides as the reference DNA sequence with the exception that all occurrences of the nitrogenous base thymine are replaced with uracil, and the sugar backbone is composed of ribose instead of deoxyribose. It is understood to be a molecule that has a sequence of bases on a backbone comprised mainly of identical monomer units at defined intervals. The bases are arranged on the backbone in such a way that they can enter into a bond with a nucleic acid having a sequence of bases that are complementary to the bases of the oligonucleotide. The most common oligonucleotides have a backbone of sugar phosphate units. A distinction may be made between oligodeoxyribonucleotides, which do not have a hydroxyl group at the 2′ position, and oligoribonucleotides, which have a hydroxyl group in this position. Oligonucleotides also may include derivatives, in which the hydrogen of the hydroxyl group is replaced with organic groups, e.g., an allyl group. An oligonucleotide is a nucleic acid that includes at least two nucleotides.

One nucleic acid sequence may be “complementary” to a second nucleic acid sequence. As used herein, the terms “complementary” or “complementarity,” when used in reference to nucleic acids (i.e., a sequence of nucleotides such as an oligonucleotide or a target nucleic acid), refer to sequences that are related by base-pairing rules. For natural bases, the base pairing rules are those developed by Watson and Crick. As an example, for the sequence “T-G-A”, the complementary sequence is “A-C-T.” Complementarity can be “partial,” in which only some of the bases of the nucleic acids are matched according to the base pairing rules. Alternatively, there can be “complete” or “total” complementarity between the nucleic acids. The degree of complementarity between the nucleic acid strands has effects on the efficiency and strength of hybridization between the nucleic acid strands.

Oligonucleotides as described herein may be capable of forming hydrogen bonds with oligonucleotides having a complementary base sequence. These bases may include the natural bases such as A, G, C, T and U, as well as artificial bases. An oligonucleotide may include nucleotide substitutions. For example, an artificial or modified base may be used in place of a natural base such that the artificial base exhibits a specific interaction that is similar to the natural base.

An oligonucleotide that is complementary to another nucleic acid will “hybridize” to the nucleic acid under suitable conditions (described below). As used herein, “hybridization” or “hybridizing” refers to the process by which an oligonucleotide single strand anneals with a complementary strand through base pairing under defined hybridization conditions. “Specific hybridization” is an indication that two nucleic acid sequences share a high degree of complementarity. Specific hybridization complexes form under permissive annealing conditions and remain hybridized after any subsequent washing steps. “Hybridizing” sequences which bind under conditions of low stringency are those which bind under non-stringent conditions (6×SSC/50% formamide at room temperature) and remain bound when washed under conditions of low stringency (2×SSC, 42° C.). Hybridizing under high stringency refers to the above conditions in which washing is performed at 2×SSC, 65° C. (where SSC is 0.15M NaCl, 0.015M sodium citrate, pH 7.2).

### EXAMPLES

Certain aspects of the disclosure are illustrated further by the following examples, which are not to be construed as limiting the disclosure in scope or spirit to the specific methods and materials described in them.

### Materials and Methods

Reagents. All oligonucleotides as described herein were purified by high performance liquid chromatography (HPLC) and purchased from Integrated DNA Technologies (USA). All oligonucleotides were subjected to ethanol precipitation and quantified using UV absorbance. ¹H NMR and ¹³C NMR spectra of the newly synthesized compounds were recorded on a Bruker AVANCE II+, 500 MHz NMR spectrophotometer in CDCl₃ and tetramethylsilane (TMS) was used as an internal standard. Mass spectra were recorded with an Agilent 6224 Accurate-Mass time-of-flight (TOF) liquid chromatography-mass spectrometry (LC/MS). Streptavidin-coated microspheres were purchased from Bangs Laboratories, Inc. Maleylated BSA (mBSA) and fluorescent transferrin (Tf-Alexa488) were synthesized according to previously published protocols^{15,18,39}. Valinomycin, nigericin, and monensin were purchased from Cayman Chemicals. All other reagents were purchased from Sigma-Aldrich (USA) unless otherwise specified.

TAC-Rh conjugation and sample preparation. TAC-Rh-N₃ was conjugated to D_{K}. TAC-Rh-N3 (25 μM) was added to 5 μM DBCO labeled D_{K} in 100 μL of sodium phosphate (10 mM) buffer containing KCl (100 mM) at pH 7.0. The reaction was stirred overnight at room temperature to achieve a 1:1 labeling of ssDNA with the TAC-Rh-N3. The reaction mixture was ethanol precipitated multiple times (12000 rpm for 10 min at 4° C.) to remove unreacted TAC-Rh-N3⁵. A ratio of 1:1 labeling of the TAC-Rh to the DNA was confirmed by using UV-Vis spectroscopy (FIG. 13). See FIG. 15A for gel characterization of the product.

Construction of pHlicKer^{Biotin}, pHlicKer^{RE}, pHlicKer^{EE/TGN}. Stock solution of pHlicKer derivatives were prepared at a final concentration of 10 μM by mixing D_{K} (K⁺ sensing strand), D_{D} (Atto488N strand), D_{A} (Atto647N strand) and D_{T} (targeting moiety modified strand) in an equimolar ratio in 20 mM sodium phosphate buffer, pH 5.5, containing 100 mM KCl (FIGS. 15A-15D, 16A-16B, 17A-17B, and 18A-18D). For all samples, annealing and gel characterization were performed according to a previously established protocol⁵.

In vitro spectroscopic measurements. Fluorescence spectra were recorded on a FluoroMax-4 scanning spectro-fluorometer (Horiba Scientific, Edison, NJ, USA) using previously established protocols.¹⁵ For recording the spectra, pHlicKer samples were diluted to 200 nM in UB4 buffer (20 mM HEPES, MES and sodium acetate, 140 mM NaCl/KCl, 1 mM CaCl₂ and MgCl₂) of the desired pH and K⁺ concentrations was excited at 495 nm, 560 nm and 640 nm, and the emission spectra were collected at 515-750 nm, 570-620 and 650-750 nm, respectively (FIGS. 7A-7E).

Cell culture, plasmids and transfection. Human embryonic kidney cells (HEK 293T) cells were gifts from B. Dickinson (University of Chicago). Cell line was checked for mycoplasma contamination using Hoechst-33342 staining. Cells were cultured in Dulbecco's Modified Eagle's Medium (Invitrogen Corporation, USA) containing 10% heat-inactivated fetal bovine serum (FBS) (Invitrogen Corporation, USA), 100 Uml⁻¹ penicillin and 100 μg/ml streptomycin, and maintained at 37° C. under 5% CO2. HEK 293T cells were passaged and plated at a confluency of 50-70% for transfection and intracellular measurements. Both hMSR1 and scFv-furin constructs are reported previously^{5,16}. HEK 293T cells were transiently transfected with the respective plasmids using TransIT-293 transfection reagent (MIRUS). After incubation for 4 h, the transfected medium was replaced with fresh medium. Cells were labelled 48 h post transfection. Human embryonic kidney 293 (HEK293) cell lines expressing wild-type Kv11.1 (WT) or the trafficking-deficient G601S-Kv11.1 channel proteins are described previously⁴⁰. Cells were cultured at 37° C. (5% CO₂) in MEM supplemented with 10% fetal bovine serum (Invitrogen) and geneticin.

Competition experiments. Scavenger receptor mediated endocytosis of 3W^{EE}. HEK 293T cells transfected with hMSR1 were incubated with 20 μM mBSA or BSA for 15 min and pulsed with a media containing 500 nM 3W^{EE} and 20 μM of mBSA or BSA for 1 h at 37° C. Cells were washed with 1×PBS three times and then imaged. Whole-cell intensities of 15 cells per dish in the A647 channel were quantified.

Transferrin receptor mediated endocytosis of 3W^{RE}. Two different dishes containing HEK 293T cells were prepared. The first dish was incubated with 20 μM of free Tf for 10 min at 37° C. Then the cells were pulsed with a mixture of 500 nM 3W^{RE} and 20 μM of free Tf for 30 min at 37° C. The second dish was pulsed with 500 nM 3W^{RE} alone (−free Tf) for 30 min at 37° C. Both the cells were then chased for 30 min. Cells were washed with 1×PBS three times and then imaged. Whole-cell intensities of 15 cells per dish in the A647 channel were quantified.

Co-localization and labelling experiments. Co-localization experiments were performed by following the previously reported protocols.⁵ Fluorescent transferrin (Tf-A₄₈₈) was used to specifically label EE by pulsing it for 10 min prior to imaging and to label RE with an additional chase time of 30 min⁴¹. Transient expression of TGN46-mCherry specifically labels the trans-Golgi network (TGN)⁴². Time required for the delivery of DNA devices with a transferrin aptamer and d(AT)4 tag when labelling RE and TGN, respectively, has been determined previously^{18,16,43}. Briefly, recycling endosomes are targeted by pulsing HEK 293T cells with 100 nM Tf-A₄₈₈ and 500 nM 3W^{RE} in Hank's Balanced Salt Solution (HBSS) for 10 minutes, followed by 30 min of chase in complete media at 37° C. To label EE, HEK 293T cells transiently transfected with hMSR1, were pulsed with 100 nM Tf-A₄₈₈ and 500 nM 3W^{EE} for 10 minutes and chased for 10 min. The trans-Golgi network is targeted by pulsing 3W^{TGN} to scFv-furin-transfected HEK 293T cells for 90 min, followed by 90 min chase. Crosstalk and bleed-through were recorded and discovered to be negligible between the A647 channel and organelle markers. Pearson's correlation coefficient (PCC) measures the pixel-by-pixel covariance of two images while it ranges from 0-1, and 1 indicates complete colocalization. PCCs are examined by the tool in ImageJ/Fiji 2.0.0-rc-54/1.51 h. On pixel shift, PCC values decrease significantly suggesting non-random colocalization.

Confocal imaging. Confocal images were captured with a Leica TCS SP5 II STED laser scanning confocal microscope (Leica Microsystems, Buffalo Grove, IL, USA) equipped with a ×63, 1.4 NA, oil immersion objective. Alexa Fluor 647 was excited using a He—Ne laser with a wavelength of 633 nm and recorded using hybrid detector (HyD).

In vitro bead calibration of pHlicKer. Bead calibration was performed using pHlicKer^{Biotin} labelled 1-μm streptavidin coated microspheres (Bangs Laboratories, Inc.). Briefly, streptavidin coated microspheres were incubated in a solution of 5 μM pHlicKer^{Biotin} in 20 mM sodium phosphate buffer, pH 5.5 and 140 mM NaCl and left for gentle mixing at room temperature. After 2 hours of shaking, the beads were collected by centrifuging at 5,000 rpm and stored in pH 5.5 and 150 mM NaCl. 0.05% of Tween-20 was added to prevent aggregation of beads. This binding solution was then spun down and the beads were reconstituted in clamping buffer (HEPES (20 mM), MES (20 mM), sodium acetate (20 mM), CaCl₂ (1 mM), MgCl₂ (1 mM), solutions were balanced with NaCl to maintain a constant ionic strength of 140 mM) with varying conc. of [K⁺] and pH. The bead solution was then drop casted on an imaging dish with cover slip and incubated for 30 min at 37° C. After 30 min incubation, beads were imaged on the IX83 inverted microscope.

Fluorescence imaging of beads. Bead imaging was done using IX83 inverted wide field microscope (Olympus Corporation of the Americas, Center Valley, PA, USA) using either a ×100 or ×60, 1.42 numerical aperture (NA), differential interference contrast (DIC) oil immersion objective (PLAPON) and Evolve Delta 512 EMCCD camera (Photometrics, USA), and controlled using MetaMorph Premier Ver 7.8.12.0 (Molecular Devices, LLC, USA), suitable for the fluorophores used. Alexa Fluor 488 channel images (D) were obtained using a 480/20 band-pass excitation filter, a 520/40 band-pass emission filter, and an 89016-ET-FITC/Cy3/Cy5 dichroic filter. FRET channel images (A) were obtained using the 480/20 band-pass excitation filter, 705/72 band-pass emission filter, and 89016-ET-FITC/Cy3/Cy5 dichroic filter. TAC-Rh channel images (O) were obtained using a 545/25 band-pass excitation filter, a 595/50 band-pass emission filter, and a an 89016-ET-FITC/Cy3/Cy5 dichroic filter. Alexa647N channel images (R) were acquired using 640/30 band pass excitation filter, 705/72 band pass emission filter and 89016 dichroic.

Image analysis of beads. Images were analyzed using Fiji (NIH, USA). For K⁺ and pH measurements, regions around the beads in each Alexa647N (R) image were identified and marked in the ROI plugin in ImageJ. The same regions were identified in the other channels by recalling the ROIs. Similarly, for background computation, a nearby region outside the beads was manually selected and saved as an ROI. The same regions were selected in the other channels by recalling the ROIs. Then, the inventors measured the mean fluorescence intensity in each bead in donor, acceptor, TAC-Rh (O), and Alexa Fluor 647 (R) channels, and the background intensity corresponding to that image and channel was subtracted. The two ratios of intensities (D/A and O/R) were then computed for each bead.

Calculating pH-corrected [K⁺]. The ratio intensities of D and A (D/A) was plotted as a function of pH to generate the pH calibration curve as shown in FIGS. 19A and 19B, which was fitted to a Boltzmann sigmoid equation:

$$pH = {pH}_{1 / 2} + ln [ ( ( {A}_{1} - {A}_{2} ) / ( D / A ) - ( {A}_{2} ) ) - 1 ] \times 0.1241 \qquad ( 1 )$$

where A1, A2, and pH_{1/2} represent parameters from a Boltzmann fit of the pH calibration curve, and Y represents the D/A ratio.

The pH dependence of pHlicKer's K⁺ sensing is given by two parameters: K_{d} of pHlicKer and fold-change in O/R given by the ratio of O/Rₘᵢₙ to O/Rₘₐₓ at every pH. The K_{d} at different pH points ranging from 5.5 to 7.0 was measured by fitting an exponential equation to measured K⁺ calibration curves as shown in FIG. 19B. The K_{d} of pHlicKer was plotted as a function of pH using the following equation:

$${K}_{d} = 46.96 + 548244 \times {e}^{- 1.627 \times p H} \qquad ( 2 )$$

The inventors obtained O/Rₘᵢₙ i.e., the O/R at low [K⁺], by clamping beads at 0.1 mM [K⁺] at different pH points.

$$O / {R}_{m i n} = 3.18 + [ ( - 6.01 / ( ( 1.766 ) \times \sqrt{( 3.14 / 2 )} ) ] \times [ {e}^{( - 2 \times {( ( p H - 6.25 ) / 1.766 )}^{2}} \qquad ( 3 )$$

O/Rₘₐₓ i.e., the O/R at high [K⁺] by clamping beads at 140 mM [K⁺] at different pH points.

$$O / {R}_{m ax} = 131.6 - ( 43.66 \times pH ) + ( 3.64 \times {pH}^{2} ) \qquad ( 4 )$$

pH and O/R were used to calculate K_{d}, O/Rₘₐₓ and O/Rₘᵢₙ from equations (2)-(4). Finally, K_{d}, O/Rₘᵢₙ, O/R, and O/Rₘₐₓ were substituted in the following equation to get pH-corrected free [K⁺] values:

$$[ {K}^{+} ] = {K}_{d} \times [ ( O / R - O / {R}_{mini} ) / ( O / R - O / {R}_{m ax} ) ] \qquad ( 5 )$$

In cellulo measurement of pH and [K⁺]. In cellulo clamping: pH and potassium clamping were carried out using pHlicKer^{RE} or pHlicKer^{EE/TGN} HEK 293T cells were pulsed with 250 nM pHlicKer^{RE} for 15 min and chased for 30 min at 37° C. Cells were then fixed with 200 μL of 4% paraformaldehyde for 5 min at 25° C. The fixed cells were washed with 1×PBS three times and incubated in the potassium clamping buffer of indicated pH and potassium concentration, containing 50 μM nigericin, 50 μM monensin and 20 μM valinomycin for 1 h at 37° C. Clamping buffers with various concentration of potassium ions were prepared by adding potassium positive buffer (140 mM KCl, 1 mM CaCl₂, 1 mM MgCl₂, 20 mM HEPES, MES, sodium acetate) to a K⁺ negative buffer (140 mM NaCl, 1 mM CaCl₂, 1 mM MgCl2, 20 mM HEPES, MES, sodium acetate) at the same indicated pH in different ratios. The cells were then imaged in clamping buffer using a widefield microscope.

EE and TGN pH and [K⁺] measurements were carried out using pHlicKer^{EE/TGN} in HEK 293T cells transfected with hMSR1 and scFv-furin respectively. For EEs, HEK 293T cells were pulsed with 250 nM pHlicKer^{EE} for 10 min and chased for 10 min. For TGN, HEK 293T cells were pulsed with 250 nM pHlicKer^{TGN} for 90 min and chased for 90 min. Cells were washed with 1×PBS three times and then imaged. For recycling endosomes, HEK 293T cells were pulsed with 250 nM pHlicKer^{RE} for 15 min and chased for 30 min. Cells were washed with 1×PBS three times and then imaged using a widefield microscope.

Image analysis: Images were analyzed using Fiji (NIH, USA). For organellar K⁺ and pH measurements, regions of cells containing single isolated endosomes in each Alexa647N (R) image were identified and marked in the ROI plugin in ImageJ. The same regions were identified in the other channels by recalling the ROls and appropriate correction factor for chromatic aberration if necessary. Similarly, for background computation, a nearby region outside the endosomes was manually selected and saved as an ROI. The same regions were selected in the other channels by recalling the ROls. Then, the inventors measured the mean fluorescence intensity in each endosome in D, A, TAC-Rh (O), and Alexa Fluor 647 (R) channels, and the background intensity corresponding to that cell and channel was subtracted. The two ratios of intensities (D/A and O/R) were then computed for each endosome. Mean D/A of each distribution was plotted as a function of pH to obtain the in cellulo pH calibration curve. Mean O/R of each distribution was plotted as a function of [K⁺] to generate the in cellulo K⁺calibration curve. Pseudocolor pH and K⁺ images were obtained by measuring the D/A and O/R ratios per pixel, respectively.

Calculating pH-corrected [K⁺] in early endosomes, trans-Golgi network, and recycling endosomes. To correct for pH and obtain K⁺ values, the K_{d} of pHlicKer in single endosomes was calculated based on the K_{d} calibration plot versus pH. The pH of the organelle was measured from the D/A values calibrated across pH 5.5-7.0. Donor (D) and acceptor (A) images were background subtracted by drawing a region of interest outside the cells. The D image was duplicated, and a threshold was set to create a binary mask. Background-subtracted D and A images were then multiplied with the binary mask to get processed D and A images. This processed D image was divided by the processed A image to get a pseudocolor D/A image, using the Image Calculator module of ImageJ. The pH value at every pixel was computed by applying equation (1) formulated from an in vitro pH calibration plot, using ImageJ.

The pseudocolored pH image was processed to get a K_{d} image (FIGS. 2A-2W). K_{d} of pHlicKer is a function of pH and this relation is formulated by the K_{d} calibration plot in vitro using equation (2). To convert the pH image to a K_{d} image, the background was set to a non-zero value. The pH-dependent K_{d} correction was performed according to equation (5), where O/Rₘₐₓ and O/Rₘᵢₙ were calculated from in cellulo clamping at 140 mM and 0.1 mM K⁺, respectively. Image calculations were done using the Image Calculator module in ImageJ. This image was multiplied with the binary image to bring the background value to zero.

Pharmacological drug treatments. pHlicKer labelled cells were treated with TEA (10 mM) or cisapride (10 μM) for 10 min in HBSS solution at room temperature. Cells were imaged in HBSS containing the respective blocker compounds.

Restoration of trafficking. To correct trafficking of Kv11.1 channels, HEK 293T cells expressing G601S-Kv11.1 were cultured for 24 h at 37° C. with 10 μM dofetilide and washed with PBS. These cells were then pulsed with 250 nM pHlicKer^{RE} for 15 min and chased for 30 min, washed with 1×PBS three times and then imaged in HBSS using a widefield microscope.

Electrophysiology. Kv11.1 current (I_{Kv11.1}) was measured by the whole-cell patch-clamp technique as described previously^{190,44}. The standard extracellular saline bath solution contained (in mmol/L) 137 NaCl, 4 KCl, 1.8 CaCl₂, 1 MgCl₂, 10 glucose, and 10 HEPES (pH 7.4 with NaOH). The modified extracellular saline bath solution to mimic the ionic conditions in recycling endosomes contained in (mmol/L) 90 NaCl, 30 KCl, 0.05 CaCl₂, 1 MgCl₂, 10 glucose, and 10 MES (pH 6.5 with NaOH). The intracellular pipette solution contained (in mmol/L) 130 KCl, 1 MgCl₂, 5 EGTA, 5 MgATP, and 10 HEPES (pH 7.2 with KOH). An Axopatch-200B patch clamp amplifier (Molecular Devices) was used to record membrane currents and measure capacitance. The uncompensated pipette resistance was 1-3 MΩ and series resistance was compensated between 75 and 85%. After obtaining intracellular access, only cells with membrane seal resistances >1 GΩ were used. The holding potential in experiments was −80 mV in recordings done in standard extracellular bath or −100 mV in recordings done in the modified extracellular saline bath. In the standard extracellular saline bath, peak I_{Kv11.1} was measured at a test-pulse of −50 mV for 5 s immediately following depolarizing pre-pulse voltage steps from −80 to 70 mV in 10-mV increments for 5 s. In the modified extracellular saline bath, peak I_{Kv11.1} was measured at a test-pulse of −100 mV for 5 s immediately following depolarizing pre-pulse voltage steps from −80 to 70 mV in 10-mV increments for 5 s. The voltage steps were applied every 20 s. Data are reported as I_{Kv11.1} density (peak I_{Kv11.1} normalized in each cell to its cell capacitance). Individual cell data were fit by the Boltzmann function^{19,44} to calculate the maximal current density (I_{MAX}), the midpoint potential for I_{Kv11.1} activation (V½), and slope factor, k, as defined by the mVle-fold change in I_{Kv11.1.} All voltage-clamp experiments were performed at 22° C. to 23° C. within 1 to 2 hours after cells were removed from their culture conditions. Voltage protocols and data analysis were done with pCLAMP 11.0 (Molecular Devices) and Graphpad (Prism) computer software.

### Example 1

### Preparation of K⁺ Fluorophore

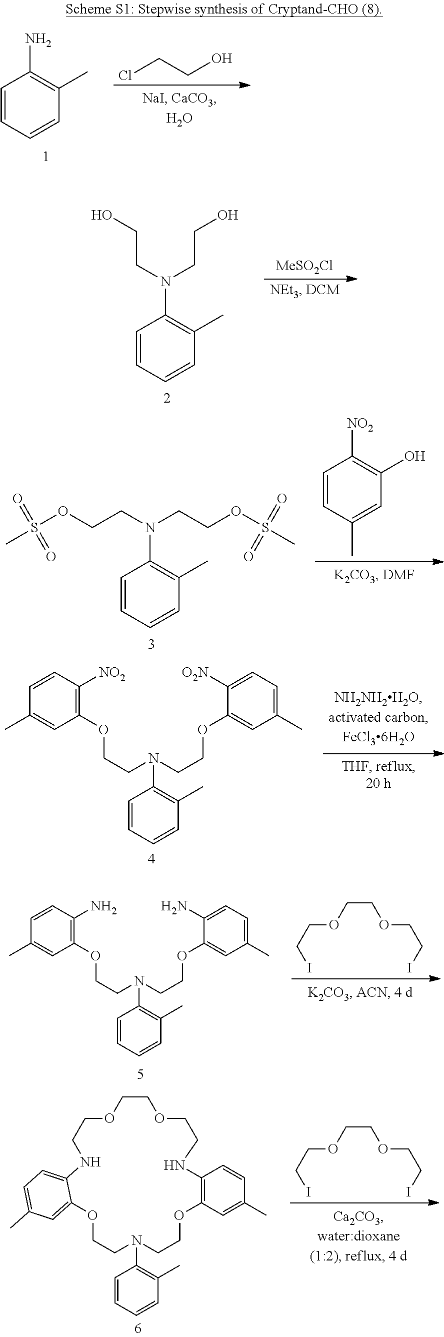

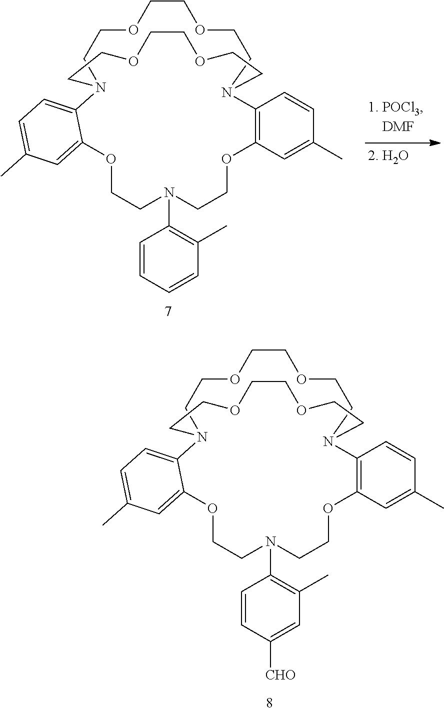

Synthesis of Compound 2: To a mixture of O-toluidine (5 g, 46.65 mmol) and chloroethanol (14 g, 93.45 mmol) suspended in 100 ml of water, CaCO₃ (14 g, 140 mmol) and NaI (catalytic amount) were added and refluxed for 24 h. After completion of the reaction, the crude product was extracted with DCM and dried over anhydrous Na₂SO₄. The solvents were removed, and the crude product was purified by column chromatography over silica gel (100-200 mesh) using 40% ethyl acetate/hexane to give the product as a yellow oily liquid. Yield 85%; ¹H NMR (500 MHZ, CDCl₃, TMS) δ (ppm): 7.18 (dd, 3H), 7.05 (dd, 1H), 3.58 (t, 4H), 3.16 (t, 4H), 3.01 (s, 2H), 2.35 (s, 3H). HRMS: m/z calculated for C₁₁H₁₇NO₂: 195.26; found: 195.11.

Synthesis of Compound 3: Compound 2 (4 g, 20.51 mmol) was dissolved in 50 mL dry DCM at 4° C. Triethylamine (5.2 g, 51.27 mmol) was added dropwise, followed by methanesulfonyl chloride (5.9 g, 51.27 mmol). After 15 minutes, the reaction was left to stir at room temperature for 2 h. The reaction mixture was neutralized with NH₄Cl solution and extracted with DCM washed successively with water, brine and dried over anhydrous Na₂SO₄. The combined organic layer was evaporated under reduced pressure to afford the desired product as a pale-yellow liquid. Yield 94%; ¹H NMR (500 MHZ, CDCl₃, TMS) δ (ppm): 7.23 (dd, 3H), 7.02 (t, 1H), 4.39 (t, 4H), 3.72 (t, 4H), 3.04 (s, 6H).

Synthesis of Compound 4: To a solution of compound 3 (2.0 g, 5.69 mmol) and 5-methyl-2-nitrophenol (2.61 g, 17.07 mmol) in 100 mL DMF, K₂CO₃ (2.36 g, 17.07 mmol) was added, and stirred at 100° C. for 24 h under inert atmosphere. The reaction mixture was then filtered and extracted with DCM. The organic layer was separated, dried over anhydrous Na₂SO₄ and concentrated. The residue was further purified by column chromatography over silica gel (100-200 mesh) using 30% ethyl acetate/hexane to give the product as a yellow solid. Yield 70%; ¹H NMR (500 MHZ, CDCl₃, TMS) δ (ppm): 7.72 (t, 2H), 7.10-7.23 (m, 4H), 6.73-6.76 (M, 4H), 4.09 (t, 4H), 3.58 (t, 4H), 2.33 (s, 6H), 2.22 (s, 3H).

Synthesis of Compound 5: Compound 4 (1.5 g, 3.22 mmol) was dissolved in 50 mL anhydrous THF, activated carbon (50 mg) and FeCl_{3.}6H₂O (0.174 g, 0.64 mmol) were added and stirred at 65° C. NH₂. NH₂. H₂O (1.6 g, 3.22 mmol) was added drop wise, and left the reaction to stir for 12 h. The reaction mixture was filtered, and the solvents were removed by distillation under reduced pressure. The residue was extracted with dichloromethane, washed successively with water, brine and dried over anhydrous Na₂SO₄. The combined organic layer was evaporated under reduced pressure to afford the desired product as a colorless liquid. Yield 98%; ¹HNMR (500 MHz, CDCl₃, TMS) δ (ppm): 7.23 (t, 2H), 7.07 (t, 1H), 6.75 (d, 1H), 6.69 (d, 1H), 6.67 (d, 3H), 6.56 (d, 2H), 4.07 (t, 4H), 3.60 (t, 4H), 2.38 (s, 3H), 2.26 (s, 6H). HRMS: m/z calculated for C₂₅H₃₁N₃O₂: 405.24; found: 406.20.

Synthesis of Compound 6: K₂CO₃ (0.82 g, 5.91 mmol) was suspended in 80 mL degassed acetonitrile and heated to reflux under argon atmosphere. Then a solution of compound 5 (0.8g, 1.97 mmol) and 1,2-bis (2-iodoethoxy) ethane (2.41 g, 6.501 mmol) in 50 mL degassed acetonitrile was added dropwise over 4 h. The resulting reaction mixture was stirred under reflux and the reaction progress was monitored by LC-MS and TLC. 4 days later, the LC-MS results indicated that the reaction completed. After cooling to room temperature, the solvent was removed under reduced pressure and the residue was extracted with DCM/H₂O and washed three times with brine. The organic layer was dried over anhydrous Na₂SO₄, then the solvent was removed by distillation. The residue was further purified by column chromatography using 2.5% methanol/DCM to give compound 6 as an off-white solid. Yield 60%; ¹H NMR (500 MHz, CDCl₃, TMS) δ (ppm): 7.21 (t, 2H), 7.08 (t, 2H), 6.90 (d, 2H), 6.70 (d, 2H), 6.46 (d, 2H), 4.03 (t, 4H), 3.85 (t, 4H), 3.70-3.79 (m, 4H), 3.67 (t, 4H), 3.26-3.37 (m, 4H), 2.15 (s, 6H), 2.05 (s, 3H).

Synthesis of Compound 7: CaCO₃ (0.288 g, 2.88 mmol) was suspended in 50 ml distilled water and is heated to reflux under argon atmosphere followed by the addition of 50 mL degased 1,4-dioxane. Then a solution of compound 6 (0.5 g, 0.961 mmol) and 1,2-bis (2-iodoethoxy) ethane (0.381 g, 0.961 mmol) in 50 mL degassed 1,4-dioxane was added dropwise over 4 h. The resulting reaction mixture was stirred under reflux and the reaction progress was monitored by LC-MS and TLC. 4 days later, the LC-MS results indicated that the reaction completed. After cooling to room temperature, the mixture was filtered, and the filtrate was condensed to 50 mL. Then the filtrate was extracted with DCM (50 mL×3). The organic layer was dried over anhydrous Na₂SO₄, then the solvent was removed by distillation. The residue was further purified by column chromatography using 3% methanol/DCM to give compound as a colorless foamy solid. Yield 45%; ¹H NMR (500 MHZ, CDCl₃, TMS) δ (ppm): 7.22 (t, 2H), 7.09 (m, 2H), 6.97 (m, 2H), 6.70 (d, 2H), 6.46 (d, 2H), 3.80 (t, 3H), 3.53-3.60 (m, 9H), 3.33-3.44 (m, 13H), 3.12-3.14 (m, 4H), 2.98-2.99 (m, 3H), 2.99 (s, 6H), 2.07 (s, 3H). HRMS: m/z calculated for C₃₇H₅₁N₃O₆: 633.38; found: 634.7.

Synthesis of Compound 8: Compound 7 (0.3 g, 0.473 mmol) dissolved in 30 mL of dry DMF, was cooled to −5-0° C. POCl₃ (0.723 g, 4.73 mmol) was added during 1 h, while the temperature was kept below 0° C. The ice bath was removed when the addition was complete. The solution was stirred at room temperature for 18 h, then warmed to 70° C. for 1 h, and poured into ice bath, basified with solid Na₂CO₃ to pH 7. Extracted with dichloromethane, dried over sodium sulfate and evaporated to yield compound 8 (0.32 g, quant.) as light-yellow oil. This oil which may still contain a small amount of DMF, was used directly for the next step without further purification.

### Example 2

### Preparation of Crosslinking-Ready K⁺ Fluorophore

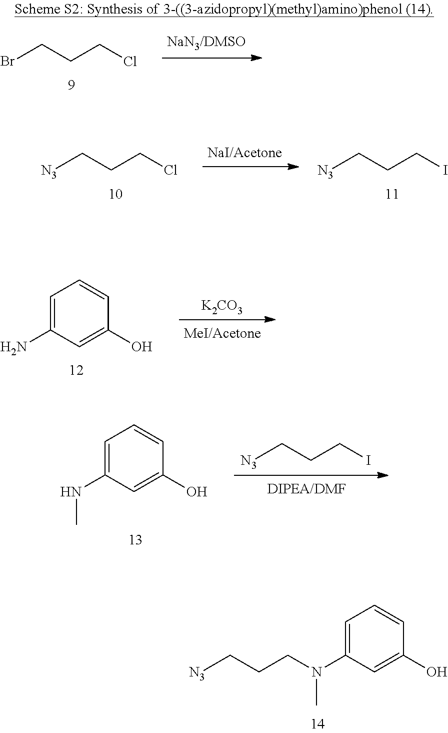

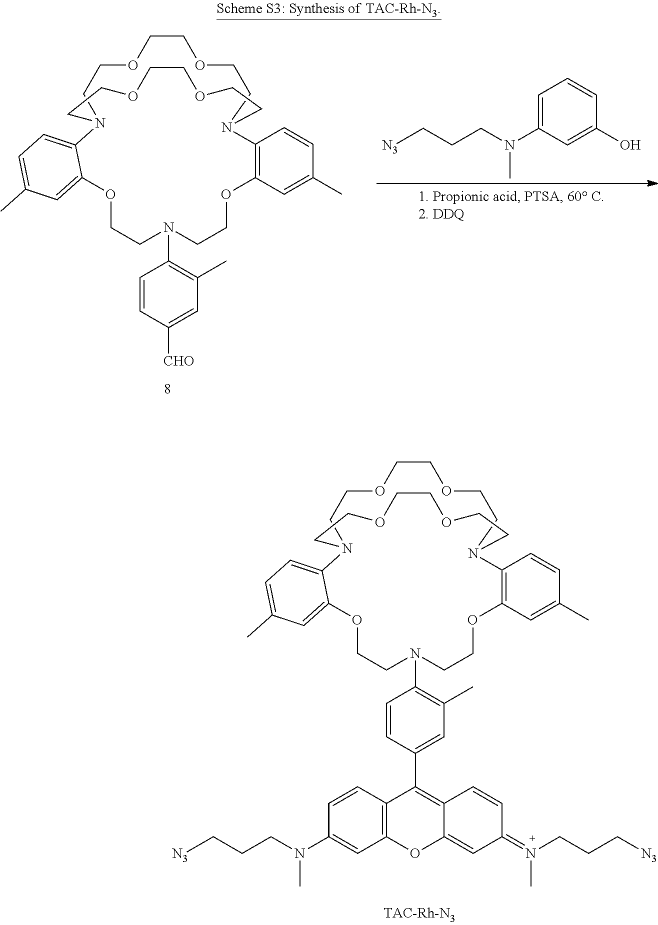

Synthesis of TAC-Rh-N₃: 3-((3-azidopropyl) (methyl) amino) phenol (14) was synthesized according to a previously reported procedure (Scheme S2).^{1 [NOW 45]}

Compound 8 (0.10 g, 0.151 mmol) and compound 14 (0.684 g, 0.332 mmol) were dissolved in 5 ml propionic acid with catalytic amount p-toluene sulfonic acid (PTSA) for 20 h at 60° C. After cooling, compound 2 was precipitated with 3M sodium acetate and the precipitated solid was collected by centrifugation, washed with water and dried, giving approximately 0.120 g of a brownish-rose colored solid, which was used immediately for subsequent reaction (see Scheme S3).

As shown in Scheme X3, the solid product (0.12 g, 0.125 mmol) was stirred with tetrachloro-1,4-benzoquinone (62 mg, 0.25 mmol) in methanol: chloroform (1:1) at ambient temperature for 15 h. Excess tetrachloro-1,4-benzoquinone was removed by filtration and reaction mixture was concentrated under reduced pressure. The residue was purified by column chromatography using 4% methanol/DCM to give compound TAC-Rh-N3 as a crimson to dark violet semisolid (overall yield ˜10%). ¹H NMR (500 MHz, CDCl₃, TMS) δ (ppm): δ 6.6-7.9 (m, 15H), 2.6-4.3 (m, 49H), 2.29 (s, 6H), 1.89 (m, 4H); HRMS: m/z calculated for C₅₈H₇₄N₁₁O₇: 1036.58; found: 1037.7.

### Example 3

### Preparation of K⁺ Fluorophore Conjugate

TAC-Rh-N3 (25 μM) was added to 5 μM DBCO labeled single-stranded nucleic acid molecule (D_{K} as shown in Table 1 below) in 100 μL of sodium phosphate (10 mM) buffer containing KCI (100 mM) at pH 7.0. The reaction was stirred overnight at room temperature to achieve a 1:1 labeling of single stranded DNA with the TAC-Rh-N3. The reaction mixture was ethanol precipitated multiple times (12000 rpm for 10 min at 4° C.) to remove unreacted TAC-Rh-N35. A ratio of 1:1 labeling of the TAC-Rh to the single stranded DNA was confirmed. FIG. 13 shows the UV-Vis absorption spectrum of SSDNA-TAC-Rh (2.97 μM, in 100 mM phosphate buffer, pH 7.2 at 25° C.) showing a 1:1 labeling of K+-sensing fluorophore (TAC-Rh) to the 25-mer ssDNA-strand. TAC-Rh conjugation was confirmed by gel electrophoresis by running a native polyacrylamide gel containing 15% (19:1 acrylamide: bis-acrylamide) in 1×TBE buffer (Tris HCl (100 mM), boric acid (89 mM), EDTA (2 mM), pH 8.3) as shown in FIG. 15A. FIG. 12 shows the spectral characteristics of pHlicKer probe. Normalized excitation and emission spectra of Alexa488N (blue), TAC-Rh (green) and Alexa647N (red). Fluorophores were chosen for their negligible spectral overlap.

### Example 4

### Preparation of Nucleic Acid Complexes of the Disclosure

Sequences used to form pHlicKer, pHlicKer^{EE}, pHlicKer^{RE}, pHlicKer^{TGN}, pHlicKer^{Biotin}, and are provided below in Table 1. D_{K}, D_{A}, D_{D} and D_{T} are used to form pHlicKer. The pHlicKer variants differed by their respective targeting molecule T, encoded on D_{T}. Strands D_{K}, D_{D}, D_{A}^{RE} and D_{T}^{RE} form pHlicKer^{RE}. D_{K}, D_{D}, D_{A}^{EE/TGN} and D_{T}^{EE/TGN} form pHlicKer^{EE/TGN}. D_{K}, D_{D}, D_{A}^{RE} and D^{Biotin} form pHlicKer^{Biotin}. 1, 2, 3 and 4 form 3WJs. D_{T}^{RE}, 1, 4 and D_{A}^{RE} form 3W^{RE}. 1, 4, D_{T}^{EE/TGN} and D_{A}^{EE/TGN} form 3W^{EE/TGN}. PHLICKER^{EE/TGN} utilizes strands SEQ ID NO:1, SEQ ID NO:2, SEQ ID NO:5, SEQ ID NO:6; PHLICKER^{RE} utilizes strands SEQ ID NO:1, SEQ ID NO:2, SEQ ID NO:3, SEQ ID NO:4; PHLICKER^{Biotin} utilizes strands SEQ ID NO:1, SEQ ID NO:2, SEQ ID NO:3, SEQ ID NO:7; 3W^{RE} utilizes strands SEQ ID NO:3, SEQ ID NO:4, SEQ ID NO:8, SEQ ID NO:11; and 3W^{EE/TGN} utilizes strands SEQ ID NO:5, SEQ ID NO:6, SEQ ID NO:8, SEQ ID NO:11.

| Strand | Sequence | Comment |
|---|---|---|
| D_{K} | 5′-DBCO-TEG- | Sensing |
| | ATCAAGGTGGCGAGAGCGACGAT | strand- |
| | CC-3′ [SEQ ID NO: 1] | conjugated to |
| | | TAC-Rh |
| D_{D} | 5′-Alexa-488-CCCCTAACCC | pH sensing |
| | CTAACCCCTAACCCCATATATAG | strand |
| | GTCAACTCTTCTCGCCACCTTGA | |
| | T-3′ [SEQ ID NO: 2] | |
| D_{A}^{RE} | 5′-CACTGCACACCAGACAGCAA | pH sensing |
| | GGATCGTCGCAGAGTTGACCT | strand of |
| | (Alexa647N)ATATATTTTGTT | RE sensor. |
| | ATGTGTTATGTGTTAT-3′ | Internal |
| | [SEQ ID NO: 3] | modification |
| | | Alexa 647 on |
| | | T with |
| | | italic and |
| | | bold. |
| D_{T}^{RE} | 5′-TTGCTGTCTGGTGTGCAGTG | RE targeting |
| | TTGATGGGGGAUCAAUCCAAGGG | strand- |
| | ACCCGGAAACGCUCCCUUACACC | modified |
| | CC-3′ [SEQ ID NO: 4] | with aptamer. |
| | | The sequence |
| | | underlined |
| | | correspond to |
| | | RNA aptamer |
| | | against hTfR |
| | | and bold |
| | | letters |
| | | indicate 2′ |
| | | fluoro |
| | | modified |
| | | bases. |
| D_{T}^{EE/TGN} | 5′-TTGCTGTCTGGTGTATATAT | |
| | AT-3′ [SEQ ID NO: 5] | |
| D_{A}^{EE/TGN} | 5′-ATATATATACACCAGACAGC | pH sensing |
| | AAGGATCGTCGCAGAGTTGACCT | strand of |
| | (Alexa647N)ATATTTTGTTAT | EE/TGN |
| | GTGTTATGTGTTAT-3′ [SEQ | sensor. |
| | ID NO: 6] | Internal |
| | | modification |
| | | Alexa 647 on |
| | | T with italic |
| | | and bold. |
| D_{T}^{Biotin} | 5′-GCGACGATCCTTGCTGTCTG | Biotin |
| | GTGTGCAGTG/3BioTEG/-3′ | modified |
| | [SEQ ID NO: 7] | strand |
| 1 | 5′-ATCAAGGTGGCGAGAGCGAC | |
| | GATCC-3′ [SEQ ID NO: 8] | |
| 2 | 5′-TTGCTGTCTGGTGTGCAGTG | |
| | TTGAT-3′ [SEQ ID NO: 9] | |
| 3 | 5′-CACTGCACACCAGACAGCAA | |
| | GGATCGTCGCAGAGTTGACCTAT | |
| | ATATTTTGTTATGTGTTATGTGT | |
| | TAT-3′ [SEQ ID NO: 10] | |
| 4 | 5′-CCCCTAACCCCTAACCCCTA | |
| | ACCCCATATATAGGTCAACTCTT | |
| | CTCGCCACCTTGAT-3′ [SEQ | |
| | ID NO: 11] | |

A three-way junction (3WJ) assembly was initially prepared to determine its stability via gel electrophoresis. Stock solutions of various assemblies were prepared at a final concentration of 10 μM by mixing single stranded nucleic acid molecules 1, 2, 3 and/or 4 in an equimolar ratio in 20 mM sodium phosphate buffer, pH 5.5, containing 100 mM KCl. The stability of three-way junction (3WJ) DNA assembly were confirmed by running a gel electrophoresis of assemblies made with different permutations and combinations of the single stranded nucleic acid molecules. FIG. 14 shows a native PAGE (10%) illustrating stepwise assembly of monomers, dimers, trimers and complete construction of 3WJ. Gels were visualized using EtBr staining. Experiments were performed in triplicate. FIG. 14 shows that the assemblies formed from formed from complementary strands displayed bands in a region that expected to show mobility shift corresponding to their molecular weight.

Based on the positive stability results shown with the 3WJ assemblies, pHlicKer^{Biotin}, pHlicKer^{RE}, pHlicKer^{EE/TGN} probe assemblies were constructed as follows. Stock solution of pHlicKer derivatives were prepared at a final concentration of 10 μM by mixing D_{K} (K⁺ sensing strand), D_{D} (Atto488N strand), D_{A} (Atto647N strand) and D_{T} (targeting moiety modified strand) in an equimolar ratio in 20 mM sodium phosphate buffer, pH 5.5, containing 100 mM KCl (FIGS. 15A-15D, FIGS. 16A-16B, FIGS. 17A-17B, and FIGS. 18A-18D). For all samples, annealing and gel characterization were performed according to a previously established protocol⁵.

Characterization of pHlicKer using gel electrophoresis. As discussed in above, TAC-Rh conjugation with ss-DNA (D_{K}) was validated by 15% Denaturing PAGE run in 1×TBE (100 mM Tris·HCl, 89 mM boric acid, and 2 mM EDTA, pH 8.3), at 150 V. Conjugation of 1 KDa (TAC-Rh) to 10 KDa (DBCO strand) causes the slow electrophoretic mobility shift of D_{K} strand in FIG. 13. Furthermore, the inventors confirmed that the lower mobility band contains TAC-Rh by fluorescence imaging in the TMR channel (excited by Epi-light and filtered by 560DF50). The D_{K} strand was purified and hybridized with the pH sensing (D_{D} and D_{A}) and targeting module (D_{T}) as described in sample preparation section. The formation of pHlicKer was validated by a gel mobility shift assay using 15% native PAGE. pHlicKer showed a lowest electrophoretic mobility than ssDNA and (D_{K}+D_{D}+D_{A}) sample. The slower mobility sample in EtBr, A488, TMR and A647 channel, indicates the formation of pHlicKer (FIG. 13).

Characterization of pHlicKer^{RE} probe assembly. As shown in FIGS. 15A-15D, the conjugation of TAC-Rh to D_{K}-DBCO strand was validated via part (A) 15% Denaturing polyacrylamide gel electrophoresis in 1×TBE, showing the conjugation. Gels were visualized in EtBr and TMR channels. Parts B) and C) validates the complete formation of pHlicKer^{RE} assembly via 15% Native polyacrylamide gel electrophoresis. Gels were visualized in EtBr, Alexa Fluor 488, TMR and Alexa Fluor 647 channels. The pHlicKer^{RE} assembly displayed a band at higher molecular weight region distinct from the assembly formed from single stranded nucleic acid molecules D_{K}, D_{D} and D_{A} (C). Experiments were performed in triplicate. D) Schematic showing the components of pHlicKer^{RE}.

Characterization of pHlicKer^{EE/TGN} probe assembly. As shown in FIGS. 16A-16B, part (A), the complete formation of pHlicKer^{EE/TGN} probe assembly was validated via 15% Native PAGE. Gels were visualized in EtBr, Alexa Fluor 488, TMR and Alexa Fluor 647 channels. pHlicKer^{EE/TGN} displayed a band at higher molecular weight region distinct from single stranded nucleic acid molecules D_{K}, D_{D} and D_{A}. Experiments were performed in triplicate. Part (B) is a schematic showing the components of pHlicKer^{RE}.

Characterization of pHlicKer^{Biotin} probe assembly. As shown in FIGS. 17A-17B, the complete formation of pHlicKer^{Biotin} probe assembly was validated via 15% Native PAGE as shown in FIG. 17A. Gels were visualized in EtBr, Alexa Fluor 488, TMR and Alexa Fluor 647 channels. pHlicKer^{Biotin} displayed a band at higher molecular weight region distinct from single stranded nucleic acid molecules D_{K}, D_{D} and D_{A}. Experiments were performed in triplicate. FIG. 17B is a schematic showing the components of pHlicKer^{Biotin}.

Characterization of 3W^{RE} and 3W^{EE/TGN} assemblies. As shown in FIGS. 18A-18D, the complete formation of 3W^{RE} and 3W^{EE/TGN} assemblies were validated via 15% Native PAGE, as shown in FIGS. 18A and 18C, respectively. Gels were visualized in EtBr and Alexa Fluor 647 channels. pHlicKer^{Biotin} displayed a band at higher molecular weight region distinct from single stranded nucleic acid molecules D_{K}, D_{D} and D_{A}. Experiments were performed in triplicate. FIGS. 18B and 18D are schematics showing the components of 3W^{RE} and 3W^{EE/TGN} assemblies, respectively.

3W^{EE/TGN} probe assemblies were constructed as follows. Stock solution of 3W^{EE/TGN} was prepared at a final concentration of 10 μM by mixing 1, 4, D_{T}^{EE/TGN} (targeting moiety modified strand) and D_{A}^{EE/TGN} (Atto647N strand) in an equimolar ratio in 20 mM sodium phosphate buffer, pH 5.5, containing 100 mM KCl (FIGS. 18A-18D). Annealing and gel characterization were performed according to a previously established protocol⁵.

Evaluation of pH sensitivity of pHlicKer. Fluorescence spectra of pHlicKer^{Biotin} immobilized on streptavidin beads were imaged in clamping buffer (HEPES (20 mM), MES (20 mM), sodium acetate (20 mM), CaCl₂ (1 mM), MgCl₂ (1 mM), solutions were balanced with NaCl to maintain a constant ionic strength of 140 mM) with different pH at 0.1 mM [K⁺]. FIG. 19A shows a plot of normalized D/A as a function of pH indicates that pHlicKer showed pH sensitivity between 6.0-7.0.

To study the pH dependent dissociation constant (K_{d}) of pHlicKer, K_{d} determined from FIG. 1C was plotted vs. pH. K_{d} values at different pH points ranging from 5.5 to 6.8 were fitted to the exponential equation. This plot reveals that K_{d} of pHlicKer increased exponentially by lowering the pH.

### Example 5

### In Vitro Characterization of Nucleic Acid Complexes of the Disclosure

K⁺ indicator on D_{K} is TAC-Rh (FIG. 1A), which has a triazacryptand core connected to rhodamine (λₑₓ=560 nm; λₑₘ=580 nm), azide linkers for conjugation and is based on TAC-Red a known K⁺ indicator¹⁰. In the absence of K⁺, rhodamine fluorescence is quenched by PeT from the nitrogen on the triazacryptand. K⁺ binding impairs PeT, relieves quenching, and turns on rhodamine fluorescence¹⁰ (FIGS. 7A-7B). At acidic pH, protonation of the amines in the triazacryptand both relieves PeT and weakens the affinity (K_{d}) of TAC-Rh for K⁺, thus making it non-trivial to extract the contribution of K⁺ to the fluorescence readout (FIG. 7C). However, given the pH of an individual organelle and knowing precisely how K_{d} changes with pH, a probe like pHlicKer allows the inventors to apply a K_{d} corrected for the acidic pH of each organelle and thereby obtain lumenal K⁺ values with single-organelle resolution. This strategy has been used to quantitate Ca²⁺ in acidic organelles¹⁷. Attaching TAC-Rh on pHlicKer did not change its K_{d}, which was still 52 mM at pH 7.0 and increases with acidity (FIGS. 7D-7E).

The reference dye, Alexa Fluor 647, on D_{A} is positioned to avoid FRET with TAC-Rh on D_{K} (FIG. 1A). Alexa Fluor 647 (λₑₓ=640 nm; λₑₘ=665 nm) was chosen because it has minimal spectral overlap with TAC-Rh and is insensitive to pH, K⁺ and other ions (FIG. 12). The 1:1 stoichiometry of Alexa Fluor 647: TAC-Rh on pHlicKer corrects for TAC-Rh intensity changes in cells due to varying probe uptake, or its non-uniform distribution. Thus, the ratio of TAC-Rh (orange channel; O) and Alexa Fluor 647 (red channel; R) intensities in pHlicKer and its variants are proportional only to pH and K⁺. Hybridization of D_{A} with D_{D} reconstitutes a known DNA-based, pH-reporter domain, called the I-switch¹⁵, which acts as a ratiometric fluorescent pH reporter (FIG. 1A). Acidic pH causes a conformational change in D_{D} and leads to high FRET between Alexa 488 (donor, D) on D_{D} with Alexa 647 (acceptor, A) on D_{A}.

The pHlicKer variants differed from each other only by their respective targeting module, T, encoded on D_{T} (FIGS. 8A-8F). pHlicKer^{EE} is transported to early endosomes (EE) by scavenger receptor-mediated endocytosis along with specific pulse and chase times¹⁶. Similarly, pHlicKer^{RE} and pHlicKer^{TGN} variants each display a different targeting module (T) that engages either the transferrin receptor or furin to target recycling endosomes (RE) or the trans-Golgi network (TGN) respectively^{16,18}. All variants were assembled from equimolar amounts of D_{A}, D_{D}, D_{K} and D_{T} incorporating the corresponding module T, and characterized by gel electrophoresis (FIGS. 15A-15D and 16A-16B).

To evaluate pHlicKer responses, biotinylated pHlicKer (pHlicKer^{Biotin}) immobilized on streptavidin-coated beads were imaged and the Alexa 488 donor-to-Alexa 647 acceptor ratio (D/A) was measured over various pH and [K⁺] values for the pH response (FIGS. 9A-9D). The TAC-Rh (O) to Alexa Fluor 647 (R) ratio (O/R) for the K⁺ response was also measured. pHlicKer reports pH from pH 5.8-7.0 with a fold change in D/A ratio of 8.5 (FIG. 1B). The D/A response is insensitive to K⁺ from 0.1-300 mM (FIG. 1B) while O/R values are sensitive to both K⁺ and acidic pH (FIG. 1C). At pH 6.8 of REs, pHlicKer response to K⁺ shows ˜3.8-fold change in O/R (FIG. 1C). Mapping O/R fold change in response to other biologically relevant cations shows that pHlicKer is specific to K⁺ and its cross reactivity to other ions is negligible (FIG. 1D).

Intracellular calibration also showed pHlicKer is responsive to endosomal pH and K⁺ (FIG. 2). The inventors targeted pHlicKer^{RE} into REs of HEK 293T cells via transferrin receptors⁵ and clamped their lumenal pH and [K⁺] using buffers of desired pH and [K⁺] containing a cocktail of ionophores (see Methods as described herein). The pH values of the clamping buffer were based on previous estimates of pH in REs, EEs and the TGN while the clamped [K⁺] were projected estimates of extremes that may be encountered in these organelles. Cells were imaged in four channels: (1) Alexa Fluor 488 donor (D) channel; (2) FRET acceptor (A) channel corresponding to Alexa Fluor 647 intensity upon exciting Alexa Fluor 488; (3) TAC-Rh orange (O) channel; and (4) Alexa Fluor 647 red (R) channel (FIGS. 2A-2D). By taking the ratio of the D image to the A image, the inventors obtained a D/A image that represents the pH map of REs at the clamped pH and K⁺ values (FIG. 2E). Similarly, the O/R image represents the K⁺ map at the clamped values (FIG. 2F). Representative O/R images clamped at pH 6.6 show that pHlicKer responds to high (140 mM; FIGS. 2E-2F) and low (0.1 mM; FIGS. 2G-2H) [K⁺]. Representative O/R images at pH 6.0 show a weaker response to [K⁺] at acidic pH (FIGS. 2E-2F and FIGS. 2I-2J). The distribution of D/A versus O/R values confirms this (FIGS. 2K-2N).

The response characteristics in cells of both the pH and K⁺-sensing modules in pHlicKer matched the in vitro performance. The fold change in D/A (FC_{D/A}) values in single endosomes clamped at pH 6.0 and 6.6 quantifies the response of the pH sensing module (FIG. 2O) while the fold change in O/R (FC_{O/R}) of endosomes clamped at 0.1 mM and 140 mM [K⁺] (FIG. 2P), and the K_{d} (FIG. 2Q) quantify the response of the K⁺ sensing module. O/R values of REs clamped at various [K⁺] reveals a K_{d} of 55 mM K⁺ at pH 6.6 (FIG. 2R). Matching FC_{D/A}, FC_{O/R} and K_{d} values in cellulo and in vitro indicates the in vitro performance characteristics of pHlicKer are quantitatively recapitulated in cells.

To obtain absolute [K⁺] in EEs, REs, and the TGN in HEK 293T cells, the inventors used a K_{d} correction factor at each pixel specified by the pH at that pixel (FIGS. 2S-2W). As an example, the inventors transformed the D/A map of REs labeled with pHlicKer^{RE} into a pH map using the in cellulo pH response profile in FIG. 2O. The pH map is further transformed into a K_{d} map by replacing each pixel in the pH map with K_{d} of pHlicKer^{RE} for K⁺ at that pH (Methods, FIG. 2Q). The K_{d} values for K⁺ at each pH is obtained from the in cellulo and in vitro K⁺ response characteristics (FIGS. 2Q-2R). The product of the equation (O/R-O/Rₘᵢₙ)/(O/Rₘₐₓ-O/R), obtained from the O/R map, with the K_{d} at each pixel finally yields the [K⁺] map. Here, O/R is the observed O/R value at a given pixel in the O/R map, and O/Rₘᵢₙ and O/Rₘₐₓ correspond to the values at 0.1 mM and 140 mM K⁺ at the pH value corresponding to the pixel of interest. [K⁺] in EE and TGN can be similarly obtained by labeling them with pHlicKer^{EE} and pHlicKer^{TGN}, respectively.

Because cell surface K⁺ channels transiting REs, EEs as well as the TGN are fully glycosylated and destined for plasma membrane insertion, the inventors used pHlicKer to test whether channels are active in these organelles. To test channel activity, the resting K⁺ levels must first be known in these organelles to predict the direction of K⁺ flow should the channels open. The inventors began by localizing pHlicKer^{EE}, pHlicKer^{TGN}, and pHlicKer^{RE} variants in EE, TGN and RE of HEK 293T cells, respectively (FIG. 3A). HEK 293T cells were used because they have low endogenous expression of voltage-gated K⁺ channels¹⁹. For evaluating targeting specificity, the corresponding pHlicKer variants carrying only the Alexa Fluor 647 reference dye (3W^{EE}, 3W^{TGN}, 3W^{RE}) were used (FIG. 3A and FIGS. 8D-8F). Because HEK 293T cells do not express scavenger receptors^{20,21,22,23,24} required for endocytosis of 3W^{EE} or pHlicKer^{EE}, the inventors transiently transfected the cells with human macrophage scavenger receptor (hMSR1)⁵ (FIGS. 10A-10B). Using previously demonstrated strategies, pHlicKer^{RE} targets the human transferrin receptor (TfR) using an RNA aptamer against TfR^{5,18}. Similarly pHlicKer^{TGN} binds a single-chain variable fragment recombinant antibody, scFv, fused to the extracellular domain of furin, via a d(AT)₄ sequence^{5,16}.

Time-dependent colocalization experiments between 3W^{EE} and an endocytic tracer such as Alexa 488-labeled transferrin (Tf-A488) revealed that a 10 min pulse of 3W^{EE} led to ˜70% colocalization in EEs (FIG. 3B). Similarly, 3W^{RE} showed ˜78% colocalization in REs of HEK293T cells (FIG. 3C). Excess unlabeled transferrin (Tf) completely competed out the uptake of 3W^{RE}, confirming that pHlicKer^{RE} was internalized via the TfR pathway (FIGS. 10C-10D). Pulsing and chasing 3W^{TGN} for 90 min each retrogradely traffics the former to the TGN as seen from ˜85% colocalization with TGN46-mCherry (FIG. 3D). Based on these results, the inventors delivered pHlicKer^{RE} to REs and measured their lumenal pH and [K⁺]. From the DIA and O/R images, the pH and [K⁺] of ˜100 REs was computed as described in FIGS. 2S-2W. The pH value of an RE versus its corresponding [K⁺] for ˜100 REs yields a scatter plot (FIG. 3E), which was converted into a color-coded, density plot for clearer visualization, referred to hereafter as a two-ion measurement (2-IM) plot²⁴ (FIG. 3F). Similar 2-IM plots for EEs and TGNs were constructed from their respective D/A and O/R images (FIGS. 3G-3I). Consistent with earlier reports^{16,25}, the 2-IM plots show the pH of REs, EEs, and TGN centered at ˜pH 6.5, 6.1 and 6.3, respectively. The lumenal [K⁺] was centered at ˜40 mM for REs, ˜19 mM for EEs and ˜102 mM for TGN (FIGS. 3F, 3H, and 3I). Interestingly, the high transmembrane [K⁺] gradient across EEs implies that K⁺ flux contributes substantially to EE membrane potential while the much lower gradient across the TGN suggests that other ions may be bigger contributors to TGN membrane potential⁵. Just as the activity of organelle-resident Cl⁻ channels and transporters alters lumenal Cl⁻ levels in organelles^{18,26}, and given that K⁺ levels in EEs, REs and the TGN are lower than the cytosol, the inventors predict that K⁺ channel activity in these three organelles will elevate their lumenal K⁺.

### Example 6

### Measurement of Activity of KCNH2-Encoded Kv11.1 Channels in TGN and in REs

In this example, the inventors have tested whether the activity of a K⁺ channel in organelles could increase its lumenal K⁺ levels in a physiological system. The functionality of the two pore K⁺ channel, TWIK2 on the cell surface of macrophages is critical for inflammation.²⁵ Recently TWIK-2 was found to also reside in REs, yet its activity in these compartments has not been tested.²⁶ The inventors could efficiently label REs in bone marrow derived macrophages (BMDMs) with pHlicKer^{RE} with a 15 minute pulse and 30 min chase (FIGS. 4A-4B). The K⁺ levels in REs of wild-type BMDMs was 52±1 mM, which dropped to 23±1 mM in those from a TWIK-2 KO mouse. Thus the presence of TWIK-2 in REs elevates the lumenal K⁺ levels suggesting this channel opens in RE membranes (FIGS. 4C-4E). Despite the ˜20 mM drop in K⁺, the lumenal pH changed negligibly (˜40 nM). This suggests that lumenal pH in REs is set by factors other than K-channels that compensate for K⁺ influx due to K-channel activity.

Furthermore, pHlicKer was used to measure the activity, if any, of KCNH2-encoded Kv11.1 channels in the TGN and in REs. Kv11.1 channels are ideal for studying whether cell surface K⁺ channels are active in organelles because they are formed by tetramerization of the prototypical pore-forming voltage-gated K⁺ channel subunits and their activity is critical for normal cardiac excitability²⁷⁻²⁹. Nearly 90% of the ˜200 KCNH2 missense mutations are linked to the deadly pro-arrhythmic long QT syndrome (LQTS)³⁰⁻³¹. Defective trafficking of selected mutant Kv11.1 channels can be corrected in cells by incubating them with drugs that bind Kv11.1 channels³⁰. The activity in TGN and Res was studied because Kv11.1 trafficking can be monitored biochemically³¹. The TGN has high membrane potential, suggesting vigorous ion transport⁵. Further, if Kv11.1 channels are produced and secreted successfully, the TGN lies early on their transport pathway. REs double as storage compartments for cell surface ion channels, after plasma membrane insertion, endocytosis and sorting^{32,33} Functional Kv11.1 channels reach these organelles later. Because HEK 293T cells do not endogenously express Kv11.1 channels, the inventors stably expressed wild type Kv11.1 channels in these cells (WT cells)¹⁹ (FIG. 5A).

First, endogenously expressed K⁺ channels were tested to see if they were active in the TGN given the high resting K⁺ levels observed therein. Hence, cells were treated with a pan voltage-gated K⁺ channel current inhibitor, i.e., tetraethyl ammonium chloride (TEA). Interestingly, K⁺ levels in TGN ([K⁺]_{TGN}) decreased significantly to ˜73 mM (FIGS. 5B-5C and FIGS. 5F-5G), indicating the functionality of TGN-resident K⁺ channels. This is consistent with the high membrane potential of the TGN, which indicates ample ion transport across these membranes. When the inventors measured [K⁺] TGN in cells expressing WT Kv_{11.1} channels, it was found that [K⁺]_{TGN} was significantly elevated to ˜117 mM (FIGS. 5D and 5H). However, in the presence of cisapride, a highly specific inhibitor of Kv11.1 channel current (I_{Kv11.1})³⁴, [K⁺]_{TGN} dropped to ˜95 mM. This palpable reduction in [K⁺]_{TGN} indicates the specific activity of Kv11.1 channels in the TGN, given their contribution to the lumenal K⁺ (FIGS. 5E and 5I).

Interestingly K⁺ levels in REs ([K⁺]_{RE}) of cells expressing WT Kv11.1 was also significant elevated to ˜78 mM (FIGS. 3F and 5B) from the ˜40 mM resting levels in HEK 293T cells lacking Kv11.1 channels (UT cells) (FIG. 3F). When WT cells were treated with TEA, [K⁺]_{RE} reverted to the resting levels of ˜37 mM (FIGS. 6B-6C). Note that TEA treatment did not change [K⁺]_{RE} in UT cells (FIG. 6D). When WT cells were treated with cisapride, [K⁺]_{RE} levels plunged to ˜39 mM, indicating that the reduced [K⁺]_{RE} in WT cells is due to inhibition of I_{Kv11.1} in REs (FIG. 6E). Together, these results indicate that Kv11.1 channels open in REs, and that channel opening changes lumenal [K⁺].

Here too, pH_{RE} did not alter appreciably when K⁺ levels changed due to channel activity, as the inventors observed in BMDMs. RE and TGN membranes contain organelle-resident pH regulators such as H⁺/Cl⁻ exchangers and Na⁺/H⁺ exchangers (NHE 9 and NHE-7).³⁵ NHEs can exchange both K⁺ and Na⁺ for H⁺. Other regulators that may be found on the TGN or RE prior to plasma membrane insertion or due to recycling respectively, are Na⁺ channels, Na⁺/K⁺ ATPase and H⁺/K⁺ ATPase.^{36,37} These players could jointly maintain luminal pH despite the influx of K⁺, just as when a cell-surface K-channel opens, even though the membrane potential and cytosolic K⁺ change, the cytosolic pH does not.

### Example 7

### Determination of Presence of Active K⁺ Channel in an Endocytic Organelle

In this example, pHlicKer was used to reveal the presence of an active K⁺ channel in an endocytic organelle. The inventors stably expressed Kv11.1 channels with a Gly601Ser missense mutation in HEK 293T cells (G601S cells) and measured the [K⁺]_{RE}. This mutation, linked to LQTS³¹, disrupts protein folding and channel trafficking³⁵, causing Kv11.1 to be retained in the ER and subsequently degraded (FIG. 20). ER retention alters the cell surface abundance of Kv11.1 and affects current density. The inventors found that [K⁺]_{RE} levels in G601S cells was ˜36 mM, far below WT cells and commensurate with UT cells. These results are consistent with a model where REs lack G601S-Kv11.1 channels, as they are retained in the ER, do not reach the cell surface and hence do not enter the recycling pathway (FIG. 6F).

### Example 8

### Evaluation of Whether G601S-Kv11.1 Mutant Channels can Recycle and are Active in Res

In this example, G601S-Kv11.1 mutant channels were tested to if they can recycle and are active in Res. The inventors cultured G601S cells with dofetilide and measured [K⁺]_{RE}. Drugs such as dofetilide block I_{Kv11.1}³⁰ and act as pharmacological chaperones that facilitate the folding of mutants like G601S-Kv11.1 (FIG. 20)³⁶. Dofetilide (10 μM) treatment for 8 h allows G601S-Kv11.1 channels to exit the ER, enter the secretory pathway and reach the cell surface³⁷ (FIG. 5A). The inventors found that after treatment and 1 h wash out of dofetilide restored [K⁺]_{RE} to levels that were comparable to WT cells (FIG. 6G), indicating that properly folded G601S-Kv11.1 channels recapitulate the trafficking and activity of WT-Kv11.1 channels even after they reach the cell surface.

Electrophysiological studies at the plasma membrane further confirm that WT-Kv11.1 channels are active under conditions that mimic those at the RE membrane (FIG. 6H). Whole-cell voltage-clamp was used to measure I_{Kv11.1} in WT and G601S cells incubated with standard extracellular saline or a modified saline that recapitulated the transmembrane ion gradients of pH, Na⁺, K⁺ and membrane potential experienced by the channel in RE membranes. Whole cell I_{Kv11.1} in standard extracellular saline was recorded using a holding potential of −80 mV. To measure the voltage-dependence of Kv11.1 channel opening, cells were pre-pulsed from −80 mV to 70 mV in 10 mV increments for 5 s immediately followed by a test pulse to −50 mV for 5 s (FIG. 6I). Because of changes in reversal potential for I_{Kv11.1} and the kinetic changes in Kv11.1 channel deactivation gating, the whole cell I_{Kv11.1} in modified extracellular saline was recorded using a holding potential of −100 mV and cells were pre-pulsed in the same way but were followed by a test pulse to −100 mV for 5 s (FIG. 6J). The peak inward and outward I_{Kv11.1} measured during the test pulse in standard and modified saline, respectively, were plotted as a function of the pre-pulse potential and the corresponding I-V relations were fitted with a Boltzmann function to calculate the I_{MAX} (FIGS. 6K-6L).

In both types of saline, WT cells have larger I_{Kv11.1} than G601S cells (FIGS. 6I and 6J), consistent with G601S-Kv11.1 channels failing to reach the cell surface. Importantly, the opening properties of WT- or G601S-Kv11.1 channels in both types of saline are similar. In fact, under conditions mimicking the RE environment, the voltage dependence of Kv11.1 channel opening (V_{1/2}) was slightly negatively shifted, indicating that some channels are open at the ionic conditions and membrane potentials experienced in REs.

In summary, using organelle-targeted nucleic acid complexes that act as pH correctable, ratiometric K⁺ reporters, the compartment-specific activity of a cell surface K⁺ channel can be addressed and could thereby show that cell surface K⁺ channels are active in organelles unlike thought previously. The pHlicKer technology combines a pH and K⁺ reporter on a single DNA scaffold, provides information on both ions with single-organelle addressability, and thereby allowed testing of a long-standing assumption that the organelle-resident fraction of cell surface K⁺ channels are inactive.

Unlike small molecules or protein-based fluorescent K⁺ reporters, which are pH sensitive and have only been used at neutral pH in the cytoplasm or nucleus³⁸, pHlicKer introduces an organelle-specific pH-correction factor that allows the inventors to extract the contribution of K⁺ in acidic organelles. pHlicKer revealed that resting K⁺ levels in EE, REs and the TGN were lower than cytosolic levels and indicated that channel activity in the membrane of these organelles would elevate lumenal K⁺. Studying the TGN and REs containing Kv11.1 channels, it was discovered that channel activity in organelles did indeed increase lumenal K⁺ levels as ions flowed down the transmembrane gradient. Cells with blocked Kv11.1 channel activity or those expressing mutant G601S-Kv11.1 channels that cannot traffic to the cell surface showed lower K⁺ levels in REs than those with functional, WT Kv11.1 channels. When trafficking was restored with dofetilide, lumenal K⁺ restored to levels commensurate with REs harbouring WT Kv11.1 channels. The inventors confirmed channel opening by electrophysiology at the plasma membrane using ionic conditions and membrane potentials that mimic those across the RE membrane.

The findings suggest that the activity of cell-surface K⁺ channels in organelles could have distinct functional consequences and hence, wider roles than previously thought. Such K⁺ channel activity could contribute to organelle membrane potential and hence, its latent ability to regulate intracellular trafficking. More broadly, because pHlicKer technology offers a practical route to map organellar K⁺ changes due to K⁺ channel activity, it can be adapted to study the compartment-specific activity other cell surface channels, or organellar K⁺ channels at an entirely new level of cellular detail. pHlicKer can also be deployed to discover new organelle-resident K⁺ channels and transporters, identify organelle-selective modulators of K⁺ channel activity, and understand the mechanisms that modulate cell-surface K⁺ channel abundance.

Some embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Additional aspects of the present disclosure are provided by the following enumerated embodiments, which may be combined in any number and in any combination that is not logically or technically inconsistent.

Embodiment 1. A method for simultaneously determining a pH and a K⁺ concentration in a sample, the method comprising:
providing a nucleic acid complex comprising:
   a first single-stranded nucleic acid molecule (D_{K}) comprising a K⁺ fluorophore conjugated to the first single-stranded nucleic acid molecule, the first single-stranded nucleic acid molecule including a first portion and a second portion;
   a second single-stranded nucleic acid molecule (D_{D}) comprising a first label of a fluorescence resonance energy transfer (FRET) pair conjugated thereto, the second single-stranded nucleic acid molecule comprising a first portion and a second portion, wherein the second portion of the second single-stranded nucleic acid molecule is complementary to the first portion of the first single-stranded nucleic acid molecule;
   a third single-stranded nucleic acid molecule (D_{A}) comprising a second label of the FRET pair conjugated thereto, the third single-stranded nucleic acid molecule comprising a first portion, a second portion, and a third portion, wherein the second portion of the third single-stranded nucleic acid molecule is complementary to the second portion of the first single-stranded nucleic acid molecule, and wherein the third portion of the third single-stranded nucleic acid molecule is at least partially complementary to the first portion of the second single-stranded nucleic acid molecule; and
   a fourth single-stranded nucleic acid molecule (D_{T}) that is at least partially complementary to the first portion of the third single-stranded nucleic acid molecule, wherein the fourth single-stranded nucleic acid molecule comprises a targeting moiety;
contacting the sample with the nucleic acid complex;
measuring an intensity of a signal produced from the contacting of the sample with the nucleic acid complex; and
determining the pH and the K⁺ concentration based on the signal.

Embodiment 2. The method of embodiment 1, wherein the determining is in early endosome, late endosome, plasma membrane, lysosome, autophagolysosome, recycling endosome, cis Golgi network, trans Golgi network, endoplasmic reticulum, peroxisomes, or secretory vesicles.

Embodiment 3. The method of embodiment 1, wherein the K⁺ fluorophore comprises a triazacryptand K(+)-selective ionophore.

Embodiment 4. The method of embodiment 3, wherein the triazacryptand K(+)-selective ionophore is coupled to rhodamine.

Embodiment 5. The method of embodiment 1, wherein the K⁺ fluorophore is coupled to the 5′-end of the first single-stranded nucleic acid molecule. Embodiment 6. The method of embodiment 5, wherein the K⁺ fluorophore comprises a formula of:

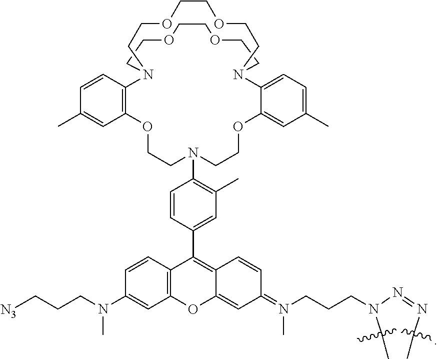

Embodiment 7. The method of embodiment 1, wherein the K⁺ fluorophore comprises a formula of:

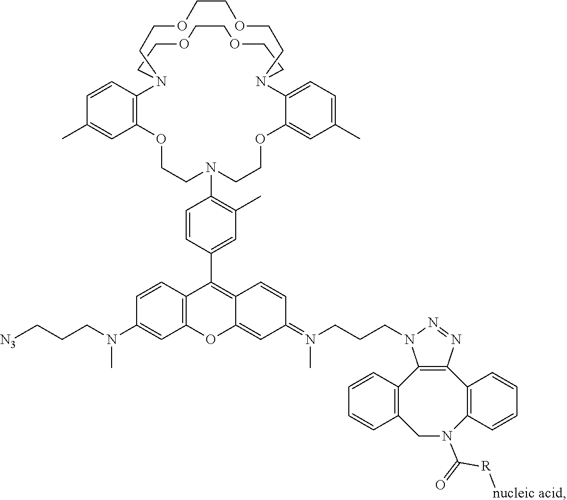

wherein R is a linker.

Embodiment 8. The method of embodiment 1, wherein the FRET pair is Alexa 647/Alexa 488.

Embodiment 9. The method embodiment 1, wherein the intensity of the signal dependent on change in pH varies as a function of the conformation of the nucleic acid complex.

Embodiment 10. The method of embodiment 9, wherein the intensity of the signal varies as a function of at least one of a distance between the first label and the second label of the FRET pair and a relative orientation of the first label and the second label of the FRET pair.

Embodiment 11. The method of embodiment 1, wherein the second single-stranded nucleic acid molecule and the third single-stranded nucleic acid molecule form an i-motif under acidic conditions.

Embodiment 12. The method of embodiment 1, wherein the second single-stranded nucleic acid molecule is capable of forming an intramolecular complex comprising two parallel-stranded C.CH+ base paired duplexes that are intercalated in an anti-parallel orientation under acidic conditions.

Embodiment 13. The method of embodiment 1, wherein the targeting moiety targets a K⁺ cell surface channel, a K⁺ cellular organelle channel, or a K⁺ transporter.

Embodiment 14. The method of embodiment 1, wherein the targeting moiety comprises a TfR aptamer, MSR1 receptor, or a scFv-furin.

Embodiment 15. The method of embodiment 1, wherein the first, the second, the third, or the fourth single-stranded nucleic acid molecule is less than 200 nucleotides, or less than 100 nucleotides, or less than 50 nucleotides.

Embodiment 16. The method of embodiment 1, wherein the determined K⁺ concentration is in a range of 0.1 mM to 1 mM, or 1 mM to 10 mM, or 10 mM to 100 mM, or 100 mM to 300 mM.

Embodiment 17. The method of embodiment 1, wherein the determined pH is in a range of 5.8 to 7.0.

Embodiment 18. A nucleic acid complex comprising:
a first single-stranded nucleic acid molecule (D_{K}) comprising a K⁺ fluorophore conjugated to the first single-stranded nucleic acid molecule, the first single-stranded nucleic acid molecule including a first portion and a second portion;
a second single-stranded nucleic acid molecule (D_{D}) comprising a first label of a fluorescence resonance energy transfer (FRET) pair conjugated thereto, the second single-stranded nucleic acid molecule comprising a first portion and a second portion, wherein the second portion of the second single-stranded nucleic acid molecule is complementary to the first portion of the first single-stranded nucleic acid molecule;
a third single-stranded nucleic acid molecule (D_{A}) comprising a second label of the FRET pair conjugated thereto, the third single-stranded nucleic acid molecule comprising a first portion, a second portion, and a third portion, wherein the second portion of the third single-stranded nucleic acid molecule is complementary to the second portion of the first single-stranded nucleic acid molecule, and wherein the third portion of the third single-stranded nucleic acid molecule is at least partially complementary to the first portion of the second single-stranded nucleic acid molecule; and
a fourth single-stranded nucleic acid molecule (D_{T}) that is at least partially complementary to the first portion of the third single-stranded nucleic acid molecule, wherein the fourth single-stranded nucleic acid molecule comprises a targeting moiety.

Embodiment 19. The nucleic acid complex of embodiment 18, wherein the K⁺ fluorophore comprises a formula of:

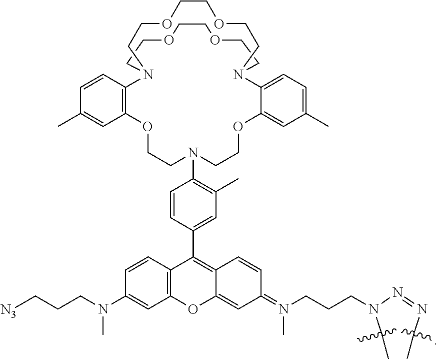

Embodiment 20. The nucleic acid complex of embodiment 18, wherein the nucleic acid complex is:
(a) pHlicKer^{RE} comprising:
   a first nucleic acid strand (D_{K}) having a sequence of 5′-DBCO-TEG-ATCAAGGTGGCGAGAGCGACGATCC-3′ [SEQ ID NO:1];
   a second nucleic acid strand (D_{D}) having a sequence of 5′-Alexa-488 -CCCCTAACCCCTAACCCCTAACCCCATATATAGGTCAACTCTTCTCGC CACCTTGAT-3′ [SEQ ID NO:2];
   a third nucleic acid strand (D_{A}^{RE}) having a sequence of 5′-CACTGCACACCAGACAGCAAGGATCGTCGCAGAGTTGACCT (Alexa64 7N) ATATATTTTGTTATGTGTTATGTGTTAT-3′ [SEQ ID NO:3]; and
   a fourth nucleic acid strand (D_{T}^{RE}) having a sequence of 5′-TTGCTGTCTGGTGTGCAGTGTTGATGGGGGAUCAAUCCAAGGGACC CGGAAACGCUCCCUUACACCCC-3′ [SEQ ID NO:4]; or
(b) pHlicKer^{EE/TGN} comprising:
   a first nucleic acid strand (D_{K}) having a sequence of 5′-DBCO-TEG-ATCAAGGTGGCGAGAGCGACGATCC-3′ [SEQ ID NO:1];
   a second nucleic acid strand (D_{D}) having a sequence of 5′-Alexa-488 -CCCCTAACCCCTAACCCCTAACCCCATATATAGGTCAACTCTTCTCGC CACCTTGAT-3′ [SEQ ID NO:2];
   a third nucleic acid strand (D_{A}^{EE/TGN}) having a sequence of 5′-ATATATATACACCAGACAGCAAGGATCGTCGCAGAGTTGACCT (Alexa 647N) ATATTTTGTTATGTGTTATGTGTTAT-3′ [SEQ ID NO:6]; and
   a fourth nucleic acid strand (D_{T}^{EE/TGN}) having a sequence of 5′-TTGCTGTCTGGTGTATATATAT-3′ [SEQ ID NO:5]; or
(c) pHlicKer^{Biotin} comprising:
   a first nucleic acid strand (D_{K}) having a sequence of 5′-DBCO-TEG-ATCAAGGTGGCGAGAGCGACGATCC-3′ [SEQ ID NO:1];
   a second nucleic acid strand (D_{D}) having a sequence of 5′-Alexa-488-CCCCTAACCCCTAACCCCTAACCCCATATATAGGTCAACTCTTCTCGC CACCTTGAT-3′ [SEQ ID NO:2];
   a third nucleic acid strand (D_{A}^{RE}) having a sequence of 5′-CACTGCACACCAGACAGCAAGGATCGTCGCAGAGTTGACCT (Alexa64 7N) ATATATTTTGTTATGTGTTATGTGTTAT-3′ [SEQ ID NO:3]; and
   a fourth nucleic acid strand (D_{T}^{Biotin}) having a sequence of 5′-GCGACGATCCTTGCTGTCTGGTGTGCAGTG/3BioTEG/−3′ [SEQ ID NO:7]; or
(d) pHlicKer^{EE} comprising:
   a first nucleic acid strand (D_{K}) having a sequence of 5′-DBCO-TEG-ATCAAGGTGGCGAGAGCGACGATCC-3′ [SEQ ID NO:1];
   a second nucleic acid strand (D_{D}) having a sequence of 5′-Alexa-488-CCCCTAACCCCTAACCCCTAACCCCATATATAGGTCAACTCTTCTCGC CACCTTGAT-3′ [SEQ ID NO:2];
   a third nucleic acid strand (D_{A}^{EE}) having a sequence of 5′-ATATATATACACCAGACAGCAAGGATCGTCGCAGAGTTGACCT (Alexa 647N) ATATTTTGTTATGTGTTATGTGTTAT-3′ [SEQ ID NO:6]; and
   a fourth nucleic acid strand (D_{T}^{EE}) having a sequence of 5′-TTGCTGTCTGGTGTATATATAT-3′ [SEQ ID NO:5]; or
(e) pHlicKer^{TGN} comprising:
   a first nucleic acid strand (D_{K}) having a sequence of 5′-DBCO-TEG-ATCAAGGTGGCGAGAGCGACGATCC-3′ [SEQ ID NO:1];
   a second nucleic acid strand (D_{D}) having a sequence of 5′-Alexa-488-CCCCTAACCCCTAACCCCTAACCCCATATATAGGTCAACTCTTCTCGC CACCTTGAT-3′ [SEQ ID NO:2];
   a third nucleic acid strand (D_{A}^{TGN}) having a sequence of 5′-ATATATATACACCAGACAGCAAGGATCGTCGCAGAGTTGACCT (Alexa 647N) ATATTTTGTTATGTGTTATGTGTTAT-3′ [SEQ ID NO:6]; and
   a fourth nucleic acid strand (D_{T}^{TGN}) having a sequence of 5′-TTGCTGTCTGGTGTATATATAT-3′ [SEQ ID NO:5]; or
(f) 3WJ comprising:
   a first nucleic acid strand (2) having a sequence of 5′-TTGCTGTCTGGTGTGCAGTGTTGAT-3′ [SEQ ID NO:9];
   a second nucleic acid strand (4) having a sequence of 5′-CCCCTAACCCCTAACCCCTAACCCCATATATAGGTCAACTCTTCTCGC CACCTTGAT-3′ [SEQ ID NO:11];
   a third nucleic acid strand (3) having a sequence of 5′-CACTGCACACCAGACAGCAAGGATCGTCGCAGAGTTGACCTATATAT TTTGTTATGTGTTATGTGTTAT-3′ [SEQ ID NO:10]; and
   a fourth nucleic acid strand (1) having a sequence of 5′-ATCAAGGTGGCGAGAGCGACGATCC-3′ [SEQ ID NO:8]; or
(g) 3W^{RE} comprising:
   a first nucleic acid strand (1) having a sequence of 5′-ATCAAGGTGGCGAGAGCGACGATCC-3′ [SEQ ID NO:8];
   a second nucleic acid strand (4) having a sequence of 5′-CCCCTAACCCCTAACCCCTAACCCCATATATAGGTCAACTCTTCTCGC CACCTTGAT-3′ [SEQ ID NO:11];
   a third nucleic acid strand (D_{A}^{RE}) having a sequence of 5′-CACTGCACACCAGACAGCAAGGATCGTCGCAGAGTTGACCT (Alexa64 7N) ATATATTTTGTTATGTGTTATGTGTTAT-3′ [SEQ ID NO:3]; and
   a fourth nucleic acid strand (D_{T}^{RE}) having a sequence of 5′-TTGCTGTCTGGTGTGCAGTGTTGATGGGGGAUCAAUCCAAGGGACC CGGAAACGCUCCCUUACACCCC-3′ [SEQ ID NO:4]; or
(h) 3W^{EE/TGN} comprising:
   a first nucleic acid strand (1) having a sequence of 5′-ATCAAGGTGGCGAGAGCGACGATCC-3′ [SEQ ID NO:8];
   a second nucleic acid strand (4) having a sequence of 5′-CCCCTAACCCCTAACCCCTAACCCCATATATAGGTCAACTCTTCTCGC CACCTTGAT-3′ [SEQ ID NO:11];
   a third nucleic acid strand (D_{A}^{EE/TGN}) having a sequence of 5′-ATATATATACACCAGACAGCAAGGATCGTCGCAGAGTTGACCT (Alexa 647N) ATATTTTGTTATGTGTTATGTGTTAT-3′ [SEQ ID NO:6]; and
   a fourth nucleic acid strand (D_{T}^{EE/TGN}) having a sequence of 5′-TTGCTGTCTGGTGTATATATAT-3′ [SEQ ID NO:5].

### REFERENCES

1. Palmer, B. F. & Clegg, D. J. Physiology and pathophysiology of potassium homeostasis. Adv. Physiol. Educ. 40, 480-490 (2016).

2. Tian, C. et al. Potassium channels: structures, diseases, and modulators. Chem. Biol. Drug Des. 83, 1-26 (2014).

3. Foo, B., Williamson, B., Young, J. C., Lukacs, G. & Shrier, A. hERG quality control and the long QT syndrome. J Physiol (Lond) 594, 2469-2481 (2016).

4. Capera, J., Serrano-Novillo, C., Navarro-Pérez, M., Cassinelli, S. & Felipe, A. The potassium channel odyssey: mechanisms of traffic and membrane arrangement. Int. J. Mol. Sci. 20, (2019).

5. Saminathan, A. et al. A DNA-based voltmeter for organelles. Nat. Nanotechnol. 16, 96-103 (2021).

6. Irannejad, R. et al. Conformational biosensors reveal GPCR signalling from endosomes. Nature 495, 534-538 (2013).

7. Tao, X., Avalos, J. L., Chen, J. & Mackinnon, R. Crystal structure of the eukaryotic strong inward-rectifier K+ channel Kir2.2 at 3.1 A resolution. Science 326, 1668-1674 (2009).

8. Sambath, K. et al. Potassium ion fluorescence probes: structures, properties and bioimaging. ChemPhotoChem 5, 317-325 (2021).

9. He, H., Mortellaro, M. A., Leiner, M. J. P., Fraatz, R. J. & Tusa, J. K. A fluorescent sensor with high selectivity and sensitivity for potassium in water. J. Am. Chem. Soc. 125, 1468-1469 (2003).

10. Padmawar, P., Yao, X., Bloch, O., Manley, G. T. & Verkman, A. S. K+ waves in brain cortex visualized using a long-wavelength K+-sensing fluorescent indicator. Nat. Methods 2, 825-827 (2005).

11. Reeves, E. P. et al. Killing activity of neutrophils is mediated through activation of proteases by K+ flux. Nature 416, 291-297 (2002).

12. Chakraborty, K., Veetil, A. T., Jaffrey, S. R. & Krishnan, Y. Nucleic Acid-Based Nanodevices in Biological Imaging. Annu. Rev. Biochem. 85, 349-373 (2016).

13. Saminathan, A., Zajac, M., Anees, P. & Krishnan, Y. Organelle-level precision with next-generation targeting technologies. Nat. Rev. Mater. (2021) doi: 10.1038/s41578-021-00396-8.

14. Krishnan, Y., Zou, J. & Jani, M. S. Quantitative imaging of biochemistry in situ and at the nanoscale. ACS Cent. Sci. 6, 1938-1954 (2020).

15. Modi, S. et al. A DNA nanomachine that maps spatial and temporal pH changes inside living cells. Nat. Nanotechnol. 4, 325-330 (2009).

16. Modi, S., Nizak, C., Surana, S., Halder, S. & Krishnan, Y. Two DNA nanomachines map pH changes along intersecting endocytic pathways inside the same cell. Nat. Nanotechnol. 8, 459-467 (2013).

17. Narayanaswamy, N. et al. A pH-correctable, DNA-based fluorescent reporter for organellar calcium. Nat. Methods 16, 95-102 (2019).

18. Saha, S., Prakash, V., Halder, S., Chakraborty, K. & Krishnan, Y. A pH-independent DNA nanodevice for quantifying chloride transport in organelles of living cells. Nat. Nanotechnol. 10, 645-651 (2015).

19. Zhou, Z. et al. Properties of HERG channels stably expressed in HEK 293 cells studied at physiological temperature. Biophys. J. 74, 230-241 (1998).

20. Surana, S., Shenoy, A. R. & Krishnan, Y. Designing DNA nanodevices for compatibility with the immune system of higher organisms. Nat. Nanotechnol. 10, 741-747 (2015).

21. Surana, S., Bhat, J. M., Koushika, S. P. & Krishnan, Y. An autonomous DNA nanomachine maps spatiotemporal pH changes in a multicellular living organism. Nat. Commun. 2, 340 (2011).

22. Bhatia, D., Surana, S., Chakraborty, S., Koushika, S. P. & Krishnan, Y. A synthetic icosahedral DNA-based host-cargo complex for functional in vivo imaging. Nat. Commun. 2, 339 (2011).

23. Veetil, A. T. et al. Cell-targetable DNA nanocapsules for spatiotemporal release of caged bioactive small molecules. Nat. Nanotechnol. 12, 1183-1189 (2017).

24. Leung, K., Chakraborty, K., Saminathan, A. & Krishnan, Y. A DNA nanomachine chemically resolves lysosomes in live cells. Nat. Nanotechnol. 14, 176-183 (2019).

25. Jani, M. S., Zou, J., Veetil, A. T. & Krishnan, Y. A DNA-based fluorescent probe maps NOS3 activity with subcellular spatial resolution. Nat. Chem. Biol. 16, 660-666 (2020).

26. Osei-Owusu, J. et al. Proton-activated chloride channel PAC regulates endosomal acidification and transferrin receptor-mediated endocytosis. Cell Rep. 34, 108683 (2021).

27. Sanguinetti, M. C., Jiang, C., Curran, M. E. & Keating, M. T. A mechanistic link between an inherited and an acquired cardiac arrhythmia: HERG encodes the IKr potassium channel. Cell 81, 299-307 (1995).

28. Keating, M. T. & Sanguinetti, M. C. Molecular and cellular mechanisms of cardiac arrhythmias. Cell 104, 569-580 (2001).

29. Sanguinetti, M. C. & Tristani-Firouzi, M. hERG potassium channels and cardiac arrhythmia. Nature 440, 463-469 (2006).

30. Zhou, Z., Gong, Q. & January, C. T. Correction of defective protein trafficking of a mutant HERG potassium channel in human long QT syndrome. Pharmacological and temperature effects. J. Biol. Chem. 274, 31123-31126 (1999).

31. Anderson, C. L. et al. Large-scale mutational analysis of Kv11.1 reveals molecular insights into type 2 long QT syndrome. Nat. Commun. 5, 5535 (2014).

32. Maxfield, F. R. & McGraw, T. E. Endocytic recycling. Nat. Rev. Mol. Cell Biol. 5, 121-132 (2004).

33. Hardel, N., Harmel, N., Zolles, G., Fakler, B. & Klöcker, N. Recycling endosomes supply cardiac pacemaker channels for regulated surface expression. Cardiovasc. Res. 79, 52-60 (2008).

34. Mohammad, S., Zhou, Z., Gong, Q. & January, C. T. Blockage of the HERG human cardiac K+ channel by the gastrointestinal prokinetic agent cisapride. Am. J. Physiol. 273, H2534-8 (1997).

35. Smith, J. L. et al. Pharmacological correction of long QT-linked mutations in KCNH2 (hERG) increases the trafficking of Kv11.1 channels stored in the transitional endoplasmic reticulum. Am J Physiol, Cell Physiol 305, C919-30 (2013).

36. Ficker, E., Obejero-Paz, C. A., Zhao, S. & Brown, A. M. The binding site for channel blockers that rescue misprocessed human long QT syndrome type 2 ether-a-gogo-related gene (HERG) mutations. J. Biol. Chem. 277, 4989-4998 (2002).

37. Qile, M. et al. LUF7244 plus Dofetilide Rescues Aberrant Kv11.1 Trafficking and Produces Functional IKv11.1. Mol. Pharmacol. 97, 355-364 (2020).

38. Bischof, H. et al. Novel genetically encoded fluorescent probes enable real-time detection of potassium in vitro and in vivo. Nat. Commun. 8, 1422 (2017).

39. Prakash, V. et al. Quantitative mapping of endosomal DNA processing by single molecule counting. Angew. Chem. Int. Ed 58, 3073-3076 (2019).

40. Delisle, B. P. et al. Thapsigargin selectively rescues the trafficking defective LQT2 channels G601S and F805C. J. Biol. Chem. 278, 35749-35754 (2003).

41. Magadán, J. G., Barbieri, M. A., Mesa, R., Stahl, P. D. & Mayorga, L. S. Rab22a regulates the sorting of transferrin to recycling endosomes. Mol. Cell. Biol. 26, 2595-2614 (2006).

42. van Galen, J. et al. Sphingomyelin homeostasis is required to form functional enzymatic domains at the trans-Golgi network. J. Cell Biol. 206, 609-618 (2014).

43. Modi, S., Halder, S., Nizak, C. & Krishnan, Y. Recombinant antibody mediated delivery of organelle-specific DNA pH sensors along endocytic pathways. Nanoscale 6, 1144-1152 (2014).

44. Delisle, B. P. et al. Intragenic suppression of trafficking-defective KCNH2 channels associated with long QT syndrome. Mol. Pharmacol. 68, 233-240 (2005).

45. Narayanaswamy, N. et al. A pH-correctable, DNA-based fluorescent reporter for organellar calcium. Nat. Methods 16, 95-102 (2019).

46. Petrecca, K., Atanasiu, R., Akhavan, A. & Shrier, A. N-linked glycosylation sites determine HERG channel surface membrane expression. J Physiol (Lond) 515 (Pt 1), 41-48 (1999).

47. Gong, Q., Anderson, C. L., January, C. T. & Zhou, Z. Role of glycosylation in cell surface expression and stability of HERG potassium channels. Am. J. Physiol. Heart Circ. Physiol. 283, H77-84 (2002).

48. Qile, M. et al. LUF7244 plus Dofetilide Rescues Aberrant Kv11.1 Trafficking and Produces Functional IKv11.1. Mol. Pharmacol. 97, 355-364 (2020).

49. Smith, J. L. et al. Trafficking-deficient hERG K⁺ channels linked to long QT syndrome are regulated by a microtubule-dependent quality control compartment in the ER. Am J Physiol, Cell Physiol 301, C75-85 (2011).

What is claimed:

1. A method for simultaneously determining a pH and a K⁺ concentration in a sample, the method comprising:
providing a nucleic acid complex comprising:
a first single-stranded nucleic acid molecule (D_{K}) comprising a K⁺ fluorophore conjugated to the first single-stranded nucleic acid molecule, the first single-stranded nucleic acid molecule including a first portion and a second portion;
a second single-stranded nucleic acid molecule (D_{D}) comprising a first label of a fluorescence resonance energy transfer (FRET) pair conjugated thereto, the second single-stranded nucleic acid molecule comprising a first portion and a second portion, wherein the second portion of the second single-stranded nucleic acid molecule is complementary to the first portion of the first single-stranded nucleic acid molecule;
a third single-stranded nucleic acid molecule (D_{A}) comprising a second label of the FRET pair conjugated thereto, the third single-stranded nucleic acid molecule comprising a first portion, a second portion, and a third portion, wherein the second portion of the third single-stranded nucleic acid molecule is complementary to the second portion of the first single-stranded nucleic acid molecule, and wherein the third portion of the third single-stranded nucleic acid molecule is at least partially complementary to the first portion of the second single-stranded nucleic acid molecule; and
a fourth single-stranded nucleic acid molecule (D_{T}) that is at least partially complementary to the first portion of the third single-stranded nucleic acid molecule, wherein the fourth single-stranded nucleic acid molecule comprises a targeting moiety;
contacting the sample with the nucleic acid complex;
measuring an intensity of a signal produced from the contacting of the sample with the nucleic acid complex; and
determining the pH and the K⁺ concentration based on the signal.

2. The method of claim 1, wherein the determining is in early endosome, late endosome, plasma membrane, lysosome, autophagolysosome, recycling endosome, cis Golgi network, trans Golgi network, endoplasmic reticulum, peroxisomes, or secretory vesicles.

3. The method of claim 1, wherein the K⁺ fluorophore comprises a triazacryptand K(+)-selective ionophore.

4. The method of claim 3, wherein the triazacryptand K(+)-selective ionophore is coupled to rhodamine.

5. The method of claim 1, wherein the K⁺ fluorophore is coupled to the 5′-end of the first single-stranded nucleic acid molecule.

6. The method of claim 5, wherein the K⁺ fluorophore comprises a formula of:

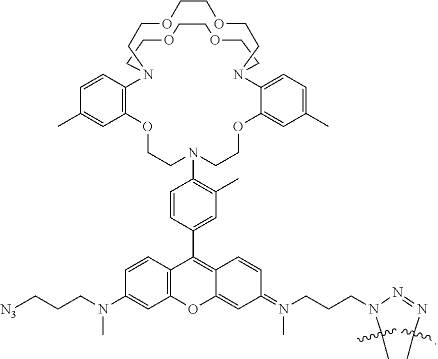

7. The method of claim 1, wherein the K⁺ fluorophore comprises a formula of: 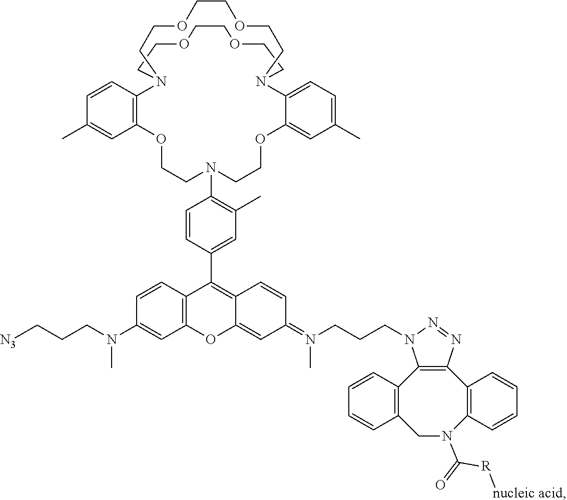
wherein R is a linker.

8. The method of claim 1, wherein the FRET pair is Alexa 647/Alexa 488.

9. The method claim 1, wherein the intensity of the signal dependent on change in pH varies as a function of the conformation of the nucleic acid complex.

10. The method of claim 9, wherein the intensity of the signal varies as a function of at least one of a distance between the first label and the second label of the FRET pair and a relative orientation of the first label and the second label of the FRET pair.

11. The method of claim 1, wherein the second single-stranded nucleic acid molecule and the third single-stranded nucleic acid molecule form an i-motif under acidic conditions.

12. The method of claim 1, wherein the second single-stranded nucleic acid molecule is capable of forming an intramolecular complex comprising two parallel-stranded C.CH+ base paired duplexes that are intercalated in an anti-parallel orientation under acidic conditions.

13. The method of claim 1, wherein the targeting moiety targets a K⁺ cell surface channel, a K⁺ cellular organelle channel, or a K⁺ transporter.

14. The method of claim 1, wherein the targeting moiety comprises a TfR aptamer, MSR1 receptor, or a scFv-furin.

15. The method of claim 1, wherein the first, the second, the third, or the fourth single-stranded nucleic acid molecule is less than 200 nucleotides.

16. The method of claim 1, wherein the determined K⁺ concentration is in a range of 0.1 mM to 1 mM. 17 The method of claim 1, wherein the determined pH is in a range of 5.8 to 7.0.

18. A nucleic acid complex comprising:
a first single-stranded nucleic acid molecule (D_{K}) comprising a K⁺ fluorophore conjugated to the first single-stranded nucleic acid molecule, the first single-stranded nucleic acid molecule including a first portion and a second portion;
a second single-stranded nucleic acid molecule (D_{D}) comprising a first label of a fluorescence resonance energy transfer (FRET) pair conjugated thereto, the second single-stranded nucleic acid molecule comprising a first portion and a second portion, wherein the second portion of the second single-stranded nucleic acid molecule is complementary to the first portion of the first single-stranded nucleic acid molecule;
a third single-stranded nucleic acid molecule (D_{A}) comprising a second label of the FRET pair conjugated thereto, the third single-stranded nucleic acid molecule comprising a first portion, a second portion, and a third portion, wherein the second portion of the third single-stranded nucleic acid molecule is complementary to the second portion of the first single-stranded nucleic acid molecule, and wherein the third portion of the third single-stranded nucleic acid molecule is at least partially complementary to the first portion of the second single-stranded nucleic acid molecule; and
a fourth single-stranded nucleic acid molecule (D_{T}) that is at least partially complementary to the first portion of the third single-stranded nucleic acid molecule, wherein the fourth single-stranded nucleic acid molecule comprises a targeting moiety.

19. The nucleic acid complex of claim 18, wherein the K⁺ fluorophore comprises a formula of:

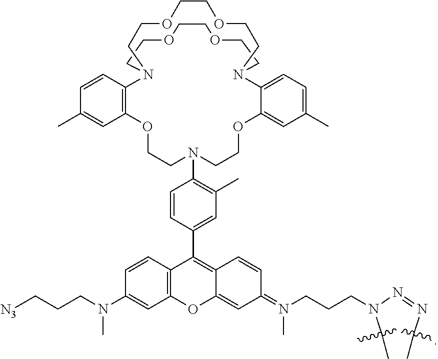

20. The nucleic acid complex of claim 18, wherein the nucleic acid complex is:
(a) pHlicKer^{RE} comprising:
a first nucleic acid strand (D_{K}) having a sequence of 5′-DBCO-TEG-ATCAAGGTGGCGAGAGCGACGATCC-3′ [SEQ ID NO:1];
a second nucleic acid strand (D_{D}) having a sequence of 5′-Alexa-488-CCCCTAACCCCTAACCCCTAACCCCATATATAGGTCAACTCTTCTCGC CACCTTGAT-3′ [SEQ ID NO:2];
a third nucleic acid strand (D_{A}^{RE}) having a sequence of 5′-CACTGCACACCAGACAGCAAGGATCGTCGCAGAGTTGACCT (Alexa64 7N) ATATATTTTGTTATGTGTTATGTGTTAT-3′ [SEQ ID NO:3]; and
a fourth nucleic acid strand (D_{T}^{RE}) having a sequence of 5′-TTGCTGTCTGGTGTGCAGTGTTGATGGGGGAUCAAUCCAAGGGACC CGGAAACGCUCCCUUACACCCC-3′ [SEQ ID NO:4]; or
(b) pHlicKer^{EE/TGN} comprising:
a first nucleic acid strand (D_{K}) having a sequence of 5′-DBCO-TEG-ATCAAGGTGGCGAGAGCGACGATCC-3′ [SEQ ID NO:1];
a second nucleic acid strand (D_{D}) having a sequence of 5′-Alexa-488-CCCCTAACCCCTAACCCCTAACCCCATATATAGGTCAACTCTTCTCGC CACCTTGAT-3′ [SEQ ID NO:2];
a third nucleic acid strand (D_{A}^{EE/TGN}) having a sequence of 5′-ATATATATACACCAGACAGCAAGGATCGTCGCAGAGTTGACCT (Alexa 647N) ATATTTTGTTATGTGTTATGTGTTAT-3′ [SEQ ID NO:6]; and
a fourth nucleic acid strand (D_{T}^{EE/TGN}) having a sequence of 5′-TTGCTGTCTGGTGTATATATAT-3′ [SEQ ID NO:5]; or
(c) pHlicKer^{Biotin} comprising:
a first nucleic acid strand (D_{K}) having a sequence of 5′-DBCO-TEG-ATCAAGGTGGCGAGAGCGACGATCC-3′ [SEQ ID NO:1];
a second nucleic acid strand (D_{D}) having a sequence of 5′-Alexa-488-CCCCTAACCCCTAACCCCTAACCCCATATATAGGTCAACTCTTCTCGC CACCTTGAT-3′ [SEQ ID NO:2];
a third nucleic acid strand (D_{A}^{RE}) having a sequence of 5′-CACTGCACACCAGACAGCAAGGATCGTCGCAGAGTTGACCT (Alexa64 7N) ATATATTTTGTTATGTGTTATGTGTTAT-3′ [SEQ ID NO:3]; and
a fourth nucleic acid strand (D_{T}^{Biotin}) having a sequence of 5′-GCGACGATCCTTGCTGTCTGGTGTGCAGTG/3BioTEG/−3′ [SEQ ID NO:7]; or
(d) pHlicKer^{EE} comprising:
a first nucleic acid strand (D_{K}) having a sequence of 5′-DBCO-TEG-ATCAAGGTGGCGAGAGCGACGATCC-3′ [SEQ ID NO:1];
a second nucleic acid strand (D_{D}) having a sequence of 5′-Alexa-488-CCCCTAACCCCTAACCCCTAACCCCATATATAGGTCAACTCTTCTCGC CACCTTGAT-3′ [SEQ ID NO:2];
a third nucleic acid strand (D_{A}^{EE}) having a sequence of 5′-ATATATATACACCAGACAGCAAGGATCGTCGCAGAGTTGACCT (Alexa 647N) ATATTTTGTTATGTGTTATGTGTTAT-3′ [SEQ ID NO:6]; and
a fourth nucleic acid strand (D_{T}^{EE}) having a sequence of 5′-TTGCTGTCTGGTGTATATATAT-3′ [SEQ ID NO:5]; or
(e) pHlicKer^{TGN} comprising:
a first nucleic acid strand (D_{K}) having a sequence of 5′-DBCO-TEG-ATCAAGGTGGCGAGAGCGACGATCC-3′ [SEQ ID NO:1];
a second nucleic acid strand (D_{D}) having a sequence of 5′-Alexa-488-CCCCTAACCCCTAACCCCTAACCCCATATATAGGTCAACTCTTCTCGC CACCTTGAT-3′ [SEQ ID NO:2];
a third nucleic acid strand (D_{A}^{TGN}) having a sequence of 5′-ATATATATACACCAGACAGCAAGGATCGTCGCAGAGTTGACCT (Alexa 647N) ATATTTTGTTATGTGTTATGTGTTAT-3′ [SEQ ID NO:6]; and
a fourth nucleic acid strand (D_{T}^{TGN}) having a sequence of 5′-TTGCTGTCTGGTGTATATATAT-3′ [SEQ ID NO:5]; or
(f) 3WJ comprising:
a first nucleic acid strand (2) having a sequence of 5′-TTGCTGTCTGGTGTGCAGTGTTGAT-3′ [SEQ ID NO:9];
a second nucleic acid strand (4) having a sequence of 5′-CCCCTAACCCCTAACCCCTAACCCCATATATAGGTCAACTCTTCTCGC CACCTTGAT-3′ [SEQ ID NO:11];
a third nucleic acid strand (3) having a sequence of 5′-CACTGCACACCAGACAGCAAGGATCGTCGCAGAGTTGACCTATATAT TTTGTTATGTGTTATGTGTTAT-3′ [SEQ ID NO:10]; and
a fourth nucleic acid strand (1) having a sequence of 5′-ATCAAGGTGGCGAGAGCGACGATCC-3′ [SEQ ID NO:8]; or
(g) 3W^{RE} comprising:
a first nucleic acid strand (1) having a sequence of 5′-ATCAAGGTGGCGAGAGCGACGATCC-3′ [SEQ ID NO:8];
a second nucleic acid strand (4) having a sequence of 5′-CCCCTAACCCCTAACCCCTAACCCCATATATAGGTCAACTCTTCTCGC CACCTTGAT-3′ [SEQ ID NO:11];
a third nucleic acid strand (D_{A}^{RE}) having a sequence of 5′-CACTGCACACCAGACAGCAAGGATCGTCGCAGAGTTGACCT (Alexa64 7N) ATATATTTTGTTATGTGTTATGTGTTAT-3′ [SEQ ID NO:3]; and
a fourth nucleic acid strand (D_{T}^{RE}) having a sequence of 5′-TTGCTGTCTGGTGTGCAGTGTTGATGGGGGAUCAAUCCAAGGGACC CGGAAACGCUCCCUUACACCCC-3′ [SEQ ID NO:4]; or
(h) 3W^{EE/TGN} comprising:
a first nucleic acid strand (1) having a sequence of 5′-ATCAAGGTGGCGAGAGCGACGATCC-3′ [SEQ ID NO:8];
a second nucleic acid strand (4) having a sequence of 5′-CCCCTAACCCCTAACCCCTAACCCCATATATAGGTCAACTCTTCTCGC CACCTTGAT-3′ [SEQ ID NO:11];
a third nucleic acid strand (D_{A}^{EE/TGN}) having a sequence of 5′-ATATATATACACCAGACAGCAAGGATCGTCGCAGAGTTGACCT (Alexa 647N) ATATTTTGTTATGTGTTATGTGTTAT-3′ [SEQ ID NO:6]; and
a fourth nucleic acid strand (D_{T}^{EE/TGN}) having a sequence of 5′-TTGCTGTCTGGTGTATATATAT-3′ [SEQ ID NO:5].


SEQUENCE LISTING (11 sequences)

SEQ ID NO: 1 - DNA, 25 bp, synthetic construct
  source 1..25: mol_type = other DNA
  modified_base 1: mod_base = OTHER; note = Dibenzocyclooctyl triethylene glycol (DBCO-TEG) on 5' end
atcaaggtggcgagagcgacgatcc

SEQ ID NO: 2 - DNA, 57 bp, synthetic construct
  source 1..57: mol_type = other DNA
  modified_base 1: mod_base = OTHER; note = Alexa 488 on 5' end
cccctaacccctaacccctaaccccatatataggtcaactcttctcgccaccttgat

SEQ ID NO: 3 - DNA, 69 bp, synthetic construct
  source 1..69: mol_type = other DNA
  modified_base 41: mod_base = OTHER; note = Alexa647N
cactgcacaccagacagcaaggatcgtcgcagagttgacctatatattttgttatgtgttatgtgttat

SEQ ID NO: 4 - DNA, 68 bp, synthetic construct
  source 1..68: mol_type = other DNA
  modified_base 32: mod_base = OTHER; note = uridine
  modified_base 36: mod_base = OTHER; note = Uridine
  modified_base 56: mod_base = OTHER; note = Uridine
  modified_base 60: mod_base = OTHER; note = Uridine
  modified_base 61: mod_base = OTHER; note = Uridine
ttgctgtctggtgtgcagtgttgatgggggatcaatccaagggacccggaaacgctcccttacacccc

SEQ ID NO: 5 - DNA, 22 bp, synthetic construct
  source 1..22: mol_type = other DNA
ttgctgtctggtgtatatatat

SEQ ID NO: 6 - DNA, 69 bp, synthetic construct
  source 1..69: mol_type = other DNA
  modified_base 43: mod_base = OTHER; note = Alexa647N
atatatatacaccagacagcaaggatcgtcgcagagttgacctatattttgttatgtgttatgtgttat

SEQ ID NO: 7 - DNA, 30 bp, synthetic construct
  source 1..30: mol_type = other DNA
  modified_base 30: mod_base = OTHER; note = 3 biotin-triethylene glycol [3BioTEG] on 3' end
gcgacgatccttgctgtctggtgtgcagtg

SEQ ID NO: 8 - DNA, 25 bp, synthetic construct
  source 1..25: mol_type = other DNA
atcaaggtggcgagagcgacgatcc

SEQ ID NO: 9 - DNA, 25 bp, synthetic construct
  source 1..25: mol_type = other DNA
ttgctgtctggtgtgcagtgttgat

SEQ ID NO: 10 - DNA, 69 bp, synthetic construct; identical to SEQ ID NO: 3
  source 1..69: mol_type = other DNA

SEQ ID NO: 11 - DNA, 56 bp, synthetic construct
  source 1..56: mol_type = other DNA
cccctaacccctaacccctaaccccatatataggtcaactcttctcgccaccttga